(19) United States
(12) Patent Application Publication
(10) Pub. No.: US 2023/0365910 A1
(43) Pub. Date: Nov. 16, 2023

(54) SYSTEMS AND METHODS FOR IN VITRO LIFE CULTURE

(71) Applicant: SHANGHAI RUIYU BIOTECH CO., LTD., Shanghai (CN)

(72) Inventors: Xuan WANG, Shanghai (CN); Rui CHEN, Shanghai (CN)

(73) Assignee: SHANGHAI RUIYU BIOTECH CO., LTD., Shanghai (CN)

(21) Appl. No.: 18/361,812

(22) Filed: Jul. 28, 2023

Related U.S. Application Data
(63) Continuation-in-part of application No. PCT/CN2021/101003, filed on Jun. 18, 2021.

(30) Foreign Application Priority Data
Jan. 29, 2021 (CN) 202110124996.7
Jan. 29, 2021 (CN) 202110126471.7

(51) Int. Cl. C12M 1/00; C12M 1/02; C12M 1/34; C12M 3/06; C12M 1/12

(52) U.S. Cl. CPC C12M 29/10; C12M 41/22; C12M 41/36; C12M 41/32; C12M 29/06; C12M 27/16; C12M 37/00; C12M 29/04

(57) ABSTRACT

The present disclosure provides an in vitro life culture system, comprising: a culture module used to cultivate a culture, the culture module including at least a culture chamber for holding a culture fluid; a culture fluid provision module used to provide the culture fluid to the culture module; and a fluid output module used to discharge the culture fluid from the culture chamber.

### CROSS-REFERENCE TO RELATED APPLICATIONS

This application is a continuation-in-part application of International Application No. PCT/CN2021/101003, filed on Jun. 18, 2021, which claims priority to Chinese Application No. 202110124996.7, filed on Jan. 29, 2021, and Chinese Application No. 202110126471.7, filed on Jan. 29, 2021, the contents of each of which are incorporated herein by reference.

### TECHNICAL FIELD

The present disclosure relates to the field of in vitro life culture, and in particular to a system and a method for in vitro life culture.

### BACKGROUND

An organoid, as a 3D cellular tissue, has considerable accuracy in the evaluation of the efficacy of oncology drugs. Currently, the culture manner of the organoid is mainly a static culture in a well plate. An operator cuts up an obtained primary sample, followed by encapsulation in a matrix gel at a low temperature, then adds a medium into the processed primary sample and places it in an incubator for incubation, and performs three fluid changes per week and a passage every 7-10 days.

Therefore, it is needed to provide an in vitro life culture system capable of automatically changing a culture fluid in a incubator to improve the existing culture manners.

### SUMMARY

One of some embodiments of the present disclosure provides an in vitro life culture system. The in vitro life culture system may include a culture module used to cultivate a culture. The culture module may include at least a culture chamber for holding a culture fluid. The in vitro life culture system may also include a culture fluid provision module used to provide the culture fluid to the culture module. The in vitro life culture system may further include a fluid output module used to discharge the culture fluid from the culture chamber.

In some embodiments, the in vitro life culture system system may further include a culture fluid circulation module used to achieve a circulation of the culture fluid in the culture chamber.

In some embodiments, the culture fluid circulation module may include a first exchange unit located outside the culture chamber, and the first exchange unit may be configured to receive the culture fluid flowing out of the culture chamber, and perform a component exchange on the culture fluid.

In some embodiments, the first exchange unit may include a first exchange chamber, a second exchange chamber, and a membrane assembly provided between the first exchange chamber and the second exchange chamber, and the membrane assembly may be used to retain or permeate at least a portion of components in the culture fluid.

In some embodiments, the first exchange chamber may include a first interface and a second interface, and the first interface may be connected with the culture chamber and the second interface being connected with at least one of the culture fluid provision module or the culture chamber. The first interface may be used to receive the culture fluid flowing into the first exchange chamber. The second interface may be used for the culture fluid flowing from the first exchange chamber out.

In some embodiments, the fluid output module may further include a collection unit, and the second exchange chamber may be connected with the collection unit.

In some embodiments, a metabolite concentration detection unit may be provided in the first exchange chamber for detecting a metabolite concentration of the culture fluid in the first exchange chamber.

In some embodiments, the first exchange unit may open or close the second interface based on a detection result of the metabolite concentration detection unit.

In some embodiments, the culture fluid circulation module may further include a power unit, and the power unit may be used to control at least one of a flow rate of the culture fluid in the first exchange chamber or a flow rate of the culture fluid in the second exchange chamber.

In some embodiments, the flow rate of the culture fluid in the first exchange chamber may be lower than the flow rate of the culture fluid in the second exchange chamber.

In some embodiments, the culture fluid circulation module may further include a fluid replenishment unit, and the fluid replenishment unit may be used to deliver one or more components required by the culture to the first exchange unit.

In some embodiments, the fluid replenishment unit may be connected with the second exchange chamber.

In some embodiments, the culture fluid circulation module may include a second exchange unit located in the culture chamber, and the second exchange unit may be used to perform a component exchange on the culture fluid in the culture chamber and circulate at least a portion of components of the culture fluid.

In some embodiments, the second exchange unit may include a first culture chamber in the culture chamber, a second culture chamber in the culture chamber, and a first membrane assembly located between the first culture chamber and the second culture chamber, and at least a portion of the components in the culture fluid may be capable of passing through the first membrane assembly by permeation.

In some embodiments, the first culture chamber may include at least one fluid inlet and at least one fluid outlet. The at least one fluid inlet may be connected with the culture fluid provision module and the at least one fluid outlet may be selectively connected with the culture fluid provision module.

In some embodiments, the at least one fluid outlet may be further selectively connected with a collection unit.

In some embodiments, the first culture chamber may include at least one fluid inlet and the second culture chamber may include at least one fluid outlet. The at least one fluid inlet may be connected with the culture fluid provision module and the at least one the fluid outlet may be selectively connected with the culture fluid provision module.

In some embodiments, the second culture chamber may include at least one fluid inlet and at least one fluid outlet. The at least one fluid inlet may be connected with the culture fluid provision module and the at least one fluid outlet may be selectively connected with the culture fluid provision module.

In some embodiments, a third membrane assembly may be provided in the first culture chamber. The third membrane assembly may divide the first culture chamber into a first sub-culture chamber and a second sub-culture chamber. The first sub-culture chamber may include a first fluid inlet and a first fluid outlet, the culture fluid provision module may be connected with the first fluid inlet, and the second culture chamber may be connected with the first fluid outlet. The second sub-culture chamber may include a second fluid outlet, the second fluid outlet being connected with the collection unit.

In some embodiments, the culture fluid circulation module may include a fluid replenishment unit, and the fluid replenishment unit may be used to deliver one or more components required by the culture to the second exchange unit.

In some embodiments, the fluid replenishment unit may be connected with the first culture chamber for delivering the one or more components required by the culture to the first culture chamber.

In some embodiments, the culture fluid provision module may include a mixing unit. The mixing unit may include a mixing chamber, and the mixing chamber may be used to mix a nutrient solution and a gas to form the culture fluid and deliver the culture fluid to the culture module.

In some embodiments, the mixing unit may further include a fluid replenishment chamber, and the fluid replenishment chamber may be connected with the mixing chamber for delivering the nutrient solution to the mixing chamber.

In some embodiments, the mixing unit may further include a pH detection member, a dissolved oxygen detection member, and a signal detector. The pH detection member and the dissolved oxygen detection member may be provided in the mixing chamber, and the signal detector may be capable of sensing signals fed back from the pH detection member and the dissolved oxygen detection member to obtain a pH and a dissolved oxygen amount of the culture fluid in the mixing chamber.

In some embodiments, the pH detection member may include a pH electrode piece and the dissolved oxygen detection member may include a dissolved oxygen electrode piece. The pH electrode piece may be provided on an inner wall of the mixing chamber or in the mixing chamber, and the dissolved oxygen detection member may be provided on the inner wall of the mixing chamber or immersed in the culture fluid of the mixing chamber.

In some embodiments, the mixing unit may further include a gas mixing control unit connected with the mixing chamber for delivering the gas to the mixing chamber.

In some embodiments, the gas mixing control unit may be connected with at least two mixing chambers for controlling a gas concentration of the at least two of the mixing chambers.

In some embodiments, the culture module may include a temperature control unit used to control a temperature of the culture chamber to a first temperature.

In some embodiments, the first temperature may correspond to a liquefaction temperature of a support structure of the culture.

In some embodiments, the temperature control unit may be further used to control the temperature of the culture chamber to switch between the first temperature and a second temperature. The second temperature may correspond to a physiological temperature of the culture.

In some embodiments, the first temperature may be lower than the second temperature.

In some embodiments, the temperature control unit may include a refrigeration assembly and a temperature control module. The refrigeration assembly may be electrically connected with the temperature control module. The temperature control module may be used to control the refrigeration assembly to cool the temperature of the culture chamber to the first temperature.

In some embodiments, the temperature control unit may further include a heating assembly, the heating assembly may be electrically connected with the temperature control module, and the temperature control module may be used to control the heating assembly to heat the culture chamber.

In some embodiments, the heating assembly may include a plurality of heating sheets, the refrigeration assembly may include a plurality of refrigeration sheets, and energy of the plurality of heating sheets may be capable of being transferred to the plurality of refrigeration sheets.

In some embodiments, the plurality of the heating sheets may be disposed at intervals and the plurality of the refrigeration sheets may be disposed at intervals. A refrigeration sheet may be disposed between two adjacent heating sheets and a heating sheet may be disposed between two adjacent refrigeration sheets.

In some embodiments, the culture module may include a microscopic observation module. The microscopic observation module may include an observation assembly, and the observation assembly may be used to observe the culture in the culture module.

In some embodiments, the culture module may include a mixing module used to shake the culture fluid in the culture chamber.

In some embodiments, the mixing module may include a stage and a frame, the culture chamber may be placed on the stage, and the stage may be capable of driving the culture chamber to move.

In some embodiments, the culture module may further include an automatic feeding module used to automatically add the culture to the culture chamber.

In some embodiments, the automatic feeding module may further comprise an automatic feeder and an automatic feeding track. The automatic feeding track may be provided on the frame and the automatic feeder may be slidably provided on the automatic feeding track.

In some embodiments, the culture module may include a sterility control module, including a sterile work chamber, a filtration assembly, and a sterilization assembly. The culture chamber may be at least provided in the sterile work chamber. The filtration assembly may be used to filter gas passing into the sterile work chamber and the sterilization assembly may be used to sterilize the sterile work chamber.

In some embodiments, at least one of the culture module, the culture fluid provision module, the fluid output module, and the culture fluid circulation module may be a disposable consumable.

One of some embodiments of the present disclosure provides a method for controlling an in vitro life culture system. The method may include obtaining a growth condition of a culture in the culture module; and controlling the culture fluid provision module or the culture module based on the growth condition.

In some embodiments, the obtaining a growth condition of a culture in a culture module may include: obtaining an image of the culture by the microscopic observation module; and determining the growth condition of the culture based on the image and a preset algorithm.

In some embodiments, the preset algorithm may include using a machine learning model.

In some embodiments, the culture module may include an oscillating drive mechanism, and the controlling the culture fluid provision module or the culture module based on the growth condition may include: controlling a motion of the oscillating drive mechanism based on the growth condition.

In some embodiments, the controlling the culture fluid provision module or the culture module based on the growth condition may include: controlling a rate at which the culture fluid is provided by the culture fluid provision module based on the growth condition.

In some embodiments, the culture module may include the temperature control unit, and the controlling the culture fluid provision module or the culture module based on the growth condition may include: controlling the temperature control unit to heat or cool the culture based on the growth condition.

One of some embodiments of the present disclosure provides a method for controlling an in vitro life culture system. The method may include obtaining a concentration of each component of the culture fluid in the culture module; and controlling a rate at which the culture fluid is provided by the culture fluid provision module based on the concentration.

One of some embodiments of the present disclosure provides an in vitro life culture system. The in vitro life culture system may include a mixing unit, the mixing unit may include a mixing chamber, and the mixing chamber may be used to mix a nutrient solution and a gas to form the culture fluid. The in vitro life culture system may also include a culture unit, the culture unit may include a culture chamber, the culture chamber may be connected with the mixing chamber, the culture fluid in the mixing chamber may be capable of being delivered to the culture chamber, and the culture chamber may be used to hold the culture and cultivate the culture. The in vitro life culture system may further include an exchange unit, the exchange unit may include an exchange chamber and a membrane assembly, the exchange chamber may include a first exchange chamber and a second exchange chamber, the membrane assembly may be provided between the first exchange chamber and the second exchange chamber, the first exchange chamber and the second exchange chamber may be connected by the membrane assembly, the first exchange chamber may be connected with the culture chamber, the first exchange chamber may be capable of both receiving the culture fluid flowing out of the culture chamber and refluxing the culture fluid to the culture chamber or the mixing chamber, and the membrane assembly may be used to retain or permeate a portion of components of the culture fluid.

In some embodiments, the mixing unit may further include a fluid replenishment chamber connected with the mixing chamber for delivering a nutrient solution to the mixing chamber, and the fluid replenishment chamber may be capable of delivering one or more components required by the culture to the mixing chamber in a directional manner.

In some embodiments, the first exchange chamber may be provided with a first interface and a second interface, the first interface may be connected with the culture chamber, and the second interface may be connected with the mixing chamber or the culture chamber. The first exchange chamber may be capable of receiving the culture fluid flowing out of the culture chamber via the first interface, and the first exchange chamber may be capable of refluxing the culture fluid to the culture chamber or the mixing chamber via the second interface.

In some embodiments, the in vitro life culture system may further include a power unit, the power unit may be provided between the mixing chamber and the culture chamber; or the power unit may be provided between the culture chamber and the first interface; or the power unit may be provided between the culture chamber or the mixing chamber and the second interface.

One of some embodiments of the present disclosure provides an in vitro life culture system. The in vitro life culture system may include a mixing unit, the mixing unit may include a mixing chamber, and the mixing chamber may be used to mix a nutrient solution and a gas to form a culture fluid. The in vitro life culture system may also include a culture unit, the culture unit may include a culture chamber, the culture chamber may be connected with the mixing chamber, the culture fluid in the mixing chamber may be capable of being delivered to the culture chamber, and the culture chamber may be used to hold the culture and cultivate the culture. The in vitro life culture system may further include an exchange unit, the exchange unit may include an exchange chamber and a membrane assembly, the exchange chamber may include a first exchange chamber and a second exchange chamber, the membrane assembly may be provided between the first exchange chamber and the second exchange chamber, and the first exchange chamber and the second exchange chamber may be connected through the membrane assembly, the culture chamber and the mixing chamber may both be connected with the first exchange chamber, the first exchange chamber may be capable of both receiving the culture fluid flowing out of the culture chamber and delivering the culture fluid to the mixing chamber or the culture chamber, and the membrane assembly may be used to retain or permeate a portion of components in the culture fluid. The in vitro life culture system may further include a fluid replenishment unit connected with the second exchange chamber for delivering a nutrient solution to the second exchange chamber, the fluid replenishment unit may be capable of delivering one or more components required by the culture to the second exchange chamber in a directional manner, and at least a portion of the components in the nutrient solution may be capable of permeating through the membrane assembly into the first exchange chamber.

In some embodiments, the first exchange chamber may be provided with a first interface and a second interface, the first interface may be connected with the culture chamber, and the second interface may be connected with at least one of the mixing chamber or the culture chamber. The culture fluid in the culture chamber may be capable of flowing into the first exchange chamber via the first interface, and the culture fluid in the first exchange chamber may be capable of flowing into the mixing chamber or the culture chamber via the second interface.

In some embodiments, the in vitro life culture system may further include a power unit. The power unit may be provided between the mixing chamber and the culture chamber, the power unit may be provided between the culture chamber and the first interface, and the power unit may be provided between the mixing chamber and the second interface or between the culture chamber and the second interface.

In some embodiments, the in vitro life culture system may further include a collection unit. The collection unit may be connected with the second exchange chamber for collecting a fluid in the second exchange chamber.

In some embodiments, at least one of the mixing unit, the culture chamber, the exchange unit, and the collection unit may be a disposable consumable.

In some embodiments, a plurality of the culture chambers may be connected with the mixing chamber; each of the plurality of culture chambers may be connected with one exchange unit, or the plurality of culture chambers may be connected with the one exchange unit.

One of some embodiments of the present disclosure provides an in vitro life culture system. The in vitro life culture system may include a mixing unit, the mixing unit may include a mixing chamber, and the mixing chamber may be used to mix a nutrient solution and a gas to form a culture fluid. The in vitro life culture system may also include a culture unit, the culture unit may include a culture chamber, the culture chamber may be connected with the mixing chamber, the culture fluid in the mixing chamber may be capable of being delivered to the culture chamber, and the culture chamber may be used to hold the culture and cultivate the culture. The in vitro life culture system may further include a temperature control unit, the temperature control unit may include a refrigeration assembly and a temperature control module, the refrigeration assembly may be electrically connected with the temperature control module, the temperature control module may be used to control the refrigeration assembly to cool the culture chamber to a first temperature, and the first temperature may be a liquefaction temperature of a support structure of the culture. A sample taking opening may be provided in the culture chamber, and the sample taking opening may be used to remove the culture after the support structure of the culture is liquefied.

In some embodiments, the temperature control unit may further include a heating assembly, the heating assembly may be electrically connected with the temperature control module, and the temperature control module may be used to control the heating assembly to heat the culture chamber or the mixing chamber.

In some embodiments, the heating assembly may include a plurality of heating sheets, the refrigeration assembly may include a plurality of refrigeration sheets, the plurality of the heating sheets may be disposed at intervals, the plurality of the refrigeration sheets may be disposed at intervals, a refrigeration sheet may be disposed between two adjacent heating sheets and a heating sheet may be disposed between two adjacent refrigeration sheets, and energy of the heating sheets may be capable of being delivered to the refrigeration sheets.

In some embodiments, the temperature control unit may further include a temperature detection member electrically connected with the temperature control module, and the temperature detection member may be used to detect a temperature of the culture fluid in the culture chamber.

One of some embodiments of the present disclosure provides an in vitro life culture system. The in vitro life culture system may include a mixing unit, the mixing unit may include a mixing chamber, the mixing chamber may be used to mix a nutrient solution and a gas to form a culture fluid. The in vitro life culture system may also include a culture unit, the culture unit may include a culture chamber, the culture chamber may be connected with the mixing chamber, the culture fluid in the mixing chamber may be capable of being delivered to the culture chamber, and the culture chamber may be used to hold the culture and cultivate the culture. The in vitro life culture system may further include a microscopic observation module, the microscopic observation module may include a stage and an observation assembly, at least one culture chamber may be placed on the stage, and the observation assembly may be used to observe the culture in the culture chamber. The microscopic observation module may further include a frame on which the stage is oscillatingly provided, and the stage may be capable of driving the culture chamber to oscillate synchronously.

In some embodiments, the microscopic observation module may further include an automatic feeder and an automatic feeding track, the automatic feeding track may be provided on the frame, the automatic feeder may be slidably provided on the automatic feeding track, and the automatic feeder may be used to add a sample to the culture chamber.

One of some embodiments of the present disclosure provides an in vitro life culture system. The in vitro life culture system may include a mixing unit, the mixing unit may include a mixing chamber, and the mixing chamber may be used to mix a nutrient solution and a gas to form a culture fluid. The in vitro life culture system may also include a culture unit, the culture unit may include a culture chamber, the culture chamber may be connected with the mixing chamber, the culture fluid in the mixing chamber may be capable of being delivered to the culture chamber, and the culture chamber may be used to hold the culture and cultivate the culture. The in vitro life culture system may further include a sterility control module, the sterility control module may include a sterile work chamber, a filtration assembly, and a sterilization assembly, the culture chamber may be at least provided in the sterile work chamber, the filtration assembly may be used to filter gas passing into the sterile work chamber, and the sterilization assembly may be used to sterilize the sterile work chamber.

In some embodiments, the mixing unit may further include a pH detection member, a dissolved oxygen detection member, and a signal detector, the pH detection member and the dissolved oxygen detection member may be provided in the mixing chamber, and the signal detector may be capable of sensing signals from the pH detection member and the dissolved oxygen detection member to obtain a pH and a dissolved oxygen amount of the culture fluid in the mixing chamber.

In some embodiments, the pH detection member may be a pH electrode piece and the dissolved oxygen detection member may be a dissolved oxygen electrode piece; the pH electrode piece may be provided on an inner wall of the mixing chamber or in the mixing chamber, and the dissolved oxygen detection member may be provided on the inner wall of the mixing chamber or immersed in the fluid in the mixing chamber.

In some embodiments, the mixing unit may further include a gas mixing control unit connected with the mixing chamber for delivering gas to the mixing chamber.

In some embodiments, one gas mixing control unit may be connected with a plurality of the mixing chambers simultaneously for controlling a gas concentration in the plurality of the mixing chambers.

In some embodiments, the culture unit may further include a mixing module used to shake the culture fluid in the culture chamber.

One of some embodiments of the present disclosure provides a method for controlling the in vitro life culture system. The method may include detecting a concentration of a component required by a culture in the culture chamber or determining a growth condition of the culture, and controlling a rate at which the culture fluid is delivered from the mixing chamber to the culture chamber based on a detected concentration or a determined growth condition of the culture.

One of some embodiments of the present disclosure provides a method for controlling the in vitro life culture system. The method may include detecting a concentration of a component required by a culture in the culture chamber or determining a growth condition of the culture, and controlling a rate at which the culture fluid is delivered from the mixing chamber to the culture chamber and a rate at which a nutrient solution is delivered from the fluid replenishment unit to the second exchange chamber based on a detected concentration or a determined growth condition of the culture.

One of some embodiments of the present disclosure provides an in vitro life culture system. The in vitro life culture system may include a mixing unit, the mixing unit may include a mixing chamber, the mixing chamber may be used to mix a nutrient solution and a gas to form a culture fluid. The in vitro life culture system may also include a culture unit, the culture unit may include a culture chamber in which a first membrane assembly is provided, the first membrane assembly may divide the culture chamber into a first culture chamber and a second culture chamber, the first membrane assembly may be used to retain or permeate a portion of components in the culture fluid; the first culture chamber may be connected with the mixing chamber, the mixing chamber may be capable of delivering the culture fluid to the first culture chamber, the culture fluid in the first culture chamber may be capable of reflowing into the mixing chamber, the second culture chamber may be used to hold the culture and cultivate the culture; or the first culture chamber may be connected with the mixing chamber, the mixing chamber may be capable of delivering the culture fluid to the first culture chamber, the second culture chamber may be connected with the mixing chamber, the culture fluid in the second culture chamber may be capable of reflowing into the mixing chamber, and the first culture chamber or the second culture chamber may be used to hold the culture and cultivate the culture.

In some embodiments, the first culture chamber may be provided with a fluid inlet, and the first culture chamber or the second culture chamber may be provided with a fluid outlet, and the fluid inlet and the fluid outlet may both be connected with the mixing chamber.

In some embodiments, the in vitro life culture system may further include a collection unit, the collection unit may be connected with the fluid outlet, and the collection unit may be used to collect the culture fluid discharged from the culture chamber.

In some embodiments, the in vitro life culture system may further include a three-way valve, and three connections of the three-way valve may be connected with the fluid outlet, the collection unit, and the mixing chamber via pipelines, respectively.

In some embodiments, the in vitro life culture system may further include a power unit, the power unit may be provided between the mixing chamber and the fluid inlet; or the power unit may be provided between the three-way valve and the fluid outlet.

In some embodiments, the mixing unit may further include a fluid replenishment chamber connected with the mixing chamber for delivering a nutrient solution to the mixing chamber, and the fluid replenishment chamber may be capable of delivering one or more components required by the culture to the mixing chamber in a directional manner.

In some embodiments, the first culture chamber may be provided with a fluid inlet, the fluid inlet may be connected with the mixing chamber, and the first culture chamber or the second culture chamber may be provided with a fluid outlet. The in vitro life culture system may further include an exchange unit, the exchange unit may include an exchange chamber and a second membrane assembly, the exchange chamber may include a first exchange chamber and a second exchange chamber, the second membrane assembly may be provided between the first exchange chamber and the second exchange chamber, the first exchange chamber and the second exchange chamber may be connected through the second membrane assembly, and the fluid outlet and the mixing unit may be connected with the first exchange chamber. The in vitro life culture system may further include a fluid replenishment unit connected with the second exchange chamber for delivering a replenishment solution to the second exchange chamber, the fluid replenishment unit may be capable of delivering one or more components required by the culture into the second exchange chamber in a directional manner, and a component of the replenishment solution may be capable of permeating through the second membrane assembly into the first exchange chamber.

In some embodiments, the in vitro life culture system may further include a collection unit, the collection unit may be connected with the second exchange chamber, and the collection unit may be used to collect the culture fluid discharged from the second exchange chamber.

One of some embodiments of the present disclosure provides an in vitro life culture system. The in vitro life culture system may include a mixing unit, the mixing unit may include a mixing chamber, and the mixing chamber may be used to mix a nutrient solution and a gas to form a culture fluid. The in vitro life culture system may also include a culture unit, the culture unit may include a culture chamber, the culture chamber may be provided with a first membrane assembly, the first membrane assembly may divide the culture chamber into a first culture chamber and a second culture chamber, the first culture chamber may be connected with the mixing chamber, the mixing chamber may be capable of delivering the culture fluid to the first culture chamber, and the first culture chamber may be used to hold the culture and cultivate the culture. The in vitro life culture system may further include a fluid replenishment unit, the fluid replenishment unit may be connected with the second culture chamber for providing a nutrient solution to the second culture chamber, the fluid replenishment unit may be capable of delivering one or more components required by the culture into the second exchange chamber in a directional manner, and at least a portion of components in the nutrient solution of the second culture chamber may be capable of permeating through the first membrane assembly into the first culture chamber. The culture fluid in the first culture chamber or the second culture chamber may be delivered to the mixing chamber.

In some embodiments, the in vitro life culture system may further include a collection unit, and at least the second culture chamber may be connected with the collection unit.

In some embodiments, a third membrane assembly may be provided in the first culture chamber, the third membrane assembly may divide the first culture chamber into a first sub-culture chamber and a second sub-culture chamber, the third membrane assembly may be used to retain or permeate components in the culture fluid, the third membrane assembly may also be capable of retaining the culture, and the first sub-culture chamber may be connected with the second culture chamber via the first membrane assembly. The mixing chamber may be connected with the first sub-culture chamber, the culture fluid in the mixing chamber may be capable of being delivered to the first sub-culture chamber, and the second sub-culture chamber may be used to hold the culture and cultivate the culture; or the mixing chamber may be connected with the second sub-culture chamber, the culture fluid in the mixing chamber may be capable of being delivered to the second sub-culture chamber, and the first sub-culture chamber may be used to hold the culture and cultivate the culture.

In some embodiments, a third membrane assembly may be provided in the first culture chamber, the third membrane assembly may divide the first culture chamber into a first sub-culture chamber and a second sub-culture chamber, the third membrane assembly may be used to retain or permeate components in the culture fluid, and the third membrane assembly may also be capable of retaining the culture. The first sub-culture chamber and the second sub-culture chamber may both be connected with the second culture chamber via the first membrane assembly, the mixing chamber may be connected with the first sub-culturing chamber, the culture fluid in the mixing chamber may be capable of being delivered to the first sub-culture chamber, and the second sub-culture chamber may be used to hold the culture and cultivate the culture.

In some embodiments, the first sub-culture chamber or the second sub-culture chamber may be capable of delivering the culture fluid to the mixing unit.

In some embodiments, when the mixing chamber is connected with the second sub-culture chamber, the first sub-culture chamber may be used to hold a culture and cultivate the culture, and the culture fluid in the first sub-culture chamber may be capable of being delivered to the second sub-culture chamber. When the mixing chamber is connected with the first sub-culture chamber, the second sub-culture chamber may be used to hold a culture and cultivate the culture, the culture fluid in the second sub-culture chamber may be capable of being delivered to the first sub-culture chamber.

In some embodiments, the mixing unit may further include a pH detection member, a dissolved oxygen detection member, and a signal detector, the pH detection member and the dissolved oxygen detection member may be provided in the mixing chamber, and the signal detector may be capable of sensing signals from the pH detection member and the dissolved oxygen detection member to obtain a pH and a dissolved oxygen amount of the culture fluid in the mixing chamber.

In some embodiments, the pH detection member may include a pH electrode piece and the dissolved oxygen detection member may include a dissolved oxygen electrode piece; the pH electrode piece may be provided on an inner wall of the mixing chamber or immersed in the culture fluid of the mixing chamber, and the dissolved oxygen detection member may be provided on the inner wall of the mixing chamber or immersed in the culture fluid of the mixing chamber.

In some embodiments, the mixing unit may further include a gas mixing control unit connected with the mixing chamber for delivering gas to the mixing chamber.

In some embodiments, at least one of the mixing unit, the culture chamber, and the collection unit may be a disposable consumable.

In some embodiments, a plurality of the culture chambers may be connected with the mixing chamber.

In some embodiments, the in vitro life culture system may further include a temperature control unit, the temperature control unit may include a refrigeration assembly and a temperature control module, the refrigeration assembly may be electrically connected with the temperature control module, the temperature control module may be used to control the refrigeration assembly to cool the culture chamber to a first temperature, and the first temperature may be a liquefaction temperature of a support structure of the culture. A sample taking opening may be provided in the culture chamber, and the sample taking opening may be used to remove the culture after the support structure of the culture is liquefied.

In some embodiments, the temperature control unit may further include a heating assembly, the heating assembly may be electrically connected with the temperature control module, and the temperature control module may be used to control the heating assembly to heat the culture chamber or the mixing chamber.

In some embodiments, the heating assembly may include a plurality of heating sheets, the refrigeration assembly may include a plurality of refrigeration sheets, the plurality of the heating sheets may be disposed at intervals, the plurality of the refrigeration sheets may be disposed at intervals, a refrigeration sheet may be disposed between two adjacent heating sheets and a heating sheet may be disposed between two adjacent refrigeration sheets, and energy of the heating sheets may be capable of being delivered to the refrigeration sheets.

In some embodiments, the temperature control unit may further include a temperature detection member electrically connected with the temperature control module, and the temperature detection member may be used to detect a temperature of the culture fluid in the culture chamber.

In some embodiments, the in vitro life culture system may further include a microscopic observation module, the microscopic observation module may include a stage and an observation assembly, at least one culture chamber may be placed on the stage, and the observation assembly may be used to observe the culture in the culture chamber. The microscopic observation module may further include a frame, the stage may be oscillatingly provided on the frame and the stage may be capable of driving the culture chamber to oscillate simultaneously.

In some embodiments, the microscopic observation module may further include an automatic feeder and an automatic feeding track, the automatic feeding track may be provided on the frame, the automatic feeder may be slidably provided on the automatic feeding track, and the automatic feeder may be used to add a sample to the culture chamber.

In some embodiments, the in vitro life culture system may further include a sterility control module, the sterility control module may include a sterile work chamber, a filtration assembly, and a sterilization assembly, the culture chamber may be at least provided in the sterile work chamber, the filtration assembly may be used to filter gas passing into the sterile work chamber, and the sterilization assembly may be used to sterilize the sterile work chamber.

In some embodiments, the culture unit may further include a mixing module used to shake the culture fluid in the culture chamber.

In some embodiments, one gas mixing control unit may be connected with a plurality of the mixing chambers simultaneously for controlling a gas concentration in the plurality of the mixing chambers.

One of some embodiments of the present disclosure provides a method for controlling the in vitro life culture system. The method may include detecting a concentration of a component required by a culture in the culture chamber or determining a growth condition of the culture, and controlling a rate at which the culture fluid is delivered from the mixing chamber to the culture chamber based on a detected concentration or a determined growth condition of the culture.

One of some embodiments of the present disclosure provides a method for controlling the in vitro life culture system. The method may include detecting a concentration of a component required by a culture in the first culture chamber or determining a growth condition of the culture, and controlling a rate at which the culture fluid is delivered from the mixing chamber to the first culture chamber and a rate at which a nutrient solution is delivered from the fluid replenishment unit to the second culture chamber based on a detected concentration or a determined growth condition of the culture.

### BRIEF DESCRIPTION OF THE DRAWINGS

The present disclosure is further illustrated in terms of exemplary embodiments. These exemplary embodiments are described in detail with reference to the drawings. These embodiments are not restrictive, in which the same numbering indicates the same structure, wherein:

FIG. 1 is a schematic diagram illustrating modules of an in vitro life culture system according to some embodiments of the present disclosure;

FIG. 2A is a schematic diagram illustrating a structure of an in vitro life culture system according to some embodiments of the present disclosure;

FIG. 2B is a schematic diagram illustrating a structure of an in vitro life culture system according to some embodiments of the present disclosure;

FIG. 3 is a schematic diagram illustrating a structure of an in vitro life culture system according to some embodiments of the present disclosure;

FIG. 4 is a schematic diagram illustrating a structure of an in vitro life culture system according to some embodiments of the present disclosure;

FIG. 5 is a schematic diagram illustrating a structure of an in vitro life culture system according to some embodiments of the present disclosure;

FIG. 6 is a schematic diagram illustrating a structure of an in vitro life culture system according to some embodiments of the present disclosure;

FIG. 7 is a schematic diagram illustrating a structure of an in vitro life culture system according to some embodiments of the present disclosure;

FIG. 8 is a schematic diagram illustrating a structure of an in vitro life culture system according to some embodiments of the present disclosure;

FIG. 9 is a schematic diagram illustrating a structure of an in vitro life culture system according to some embodiments of the present disclosure;

FIG. 10 is a schematic diagram illustrating a structure of an in vitro life culture system according to some embodiments of the present disclosure;

FIG. 11 is a schematic diagram illustrating a structure of an in vitro life culture system according to some embodiments of the present disclosure;

FIG. 12 is a schematic diagram illustrating a structure of an in vitro life culture system according to some embodiments of the present disclosure;

FIG. 13 is a schematic diagram illustrating a structure of an in vitro life culture system according to some embodiments of the present disclosure;

FIG. 14 is a schematic diagram illustrating a structure of a culture chamber according to some embodiments of the present disclosure;

FIG. 15 is a schematic diagram illustrating a structure of an in vitro life culture system according to some embodiments of the present disclosure;

FIG. 16 is a schematic diagram illustrating a structure of an in vitro life culture system according to some embodiments of the present disclosure;

FIG. 17 is a schematic diagram illustrating a structure of an in vitro life culture system according to some embodiments of the present disclosure;

FIG. 18 is a schematic diagram illustrating a structure of a culture chamber according to some embodiments of the present disclosure;

FIG. 19 is a schematic diagram illustrating a structure of an in vitro life culture system according to some embodiments of the present disclosure;

FIG. 20 is a schematic diagram illustrating a structure of an in vitro life culture system according to some embodiments of the present disclosure;

FIG. 21 is a schematic diagram illustrating a structure of an in vitro life culture system according to some embodiments of the present disclosure;

FIG. 22 is a schematic diagram illustrating a structure of a temperature control unit according to some embodiments of the present disclosure;

FIG. 23 is a schematic diagram illustrating a structure of a temperature control unit according to some embodiments of the present disclosure;

FIG. 24 is a schematic diagram illustrating a structure of a temperature control unit according to some embodiments of the present disclosure;

FIG. 25 is a schematic diagram illustrating a structure of a microscopic observation module according to some embodiments of the present disclosure;

FIG. 26 is a schematic diagram illustrating a structure of a microscopic observation module according to some embodiments of the present disclosure;

FIG. 27 is a schematic diagram illustrating a structure of a microscopic observation module according to some embodiments of the present disclosure;

FIG. 28 is a schematic diagram illustrating a structure of a microscopic observation module according to some embodiments of the present disclosure;

FIG. 29 is a schematic diagram illustrating a structure of a microscopic observation module according to some embodiments of the present disclosure;

FIG. 30 is a schematic diagram illustrating a structure of a sterility control module according to some embodiments of the present disclosure;

FIG. 31 is a flowchart illustrating a method for controlling an in vitro life culture system according to some embodiments of the present disclosure;

FIG. 32 is a flowchart illustrating a process for controlling a culture fluid provision module and/or a culture module based on a growth condition according to some embodiments of the present disclosure;

FIG. 33 is a schematic diagram illustrating a parameter judgment model according to some embodiments of the present disclosure;

FIG. 34 is a flowchart illustrating a method for controlling an in vitro life culture system according to some embodiments of the present disclosure;

FIG. 35 is a flowchart illustrating a method for controlling an in vitro life culture system according to some embodiments of the present disclosure.

### DETAILED DESCRIPTION

In order to more clearly illustrate the technical solutions related to the embodiments of the present disclosure, a brief introduction of the drawings referred to the description of the embodiments is provided below. Obviously, the drawings described below are only some examples or embodiments of the present disclosure. Those ordinary skilled in the art, without further creative efforts, may apply the present disclosure to other similar scenarios according to these drawings. Unless obviously obtained from the context or the context illustrates otherwise, the same numeral in the drawings refers to the same structure or operation.

It should be understood that the “system,” “device,” “unit,” and/or “module” used herein are a method to distinguish different components, elements, parts, sections or assemblies of different levels. However, if other words may achieve the same purpose, the words may be replaced by other expressions.

As used in the disclosure and the appended claims, the singular forms “a,” “an,” and “the” include plural referents unless the content clearly dictates otherwise. Generally speaking, the terms “comprise” and “include” only imply that the clearly identified steps and elements are included, and these steps and elements do not constitute an exclusive list, and the method or device may also include other steps or elements.

The flowcharts used in the present disclosure illustrate operations that systems implement according to some embodiments of the present disclosure. It should be understood that the preceding or following operations are not necessarily performed in exact order. Instead, each step may be processed in reverse order or simultaneously. Moreover, one or more other operations may be added into the flowcharts. One or more operations may be removed from the flowcharts.

The in vitro life culture system of one or more embodiments of the present disclosure may be applied to the culture of various in vitro lives. Various in vitro lives may include, but are not limited to, an ordinary human cell, an ordinary animal cell, a human organ tumor cell, an animal tumor cell, a bacterial cell, a viral cell, an antibody cell, a microbial cell population, etc. In some embodiments, the in vitro life culture system may provide a perfusion culture for an in vitro life to be cultured, such that the in vitro life is grown in a culture fluid in a flowing state.

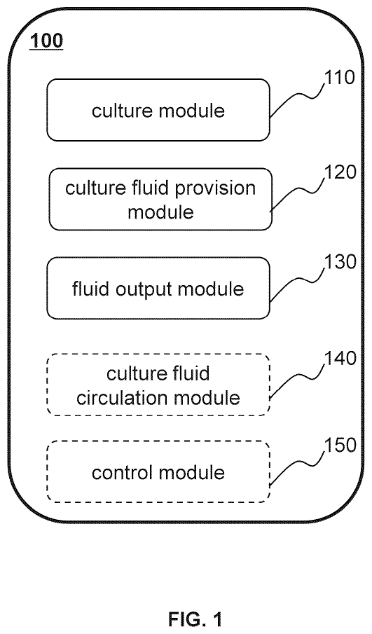

FIG. 1 is a schematic diagram illustrating modules of an in vitro life culture system 100 according to some embodiments of the present disclosure.

In some embodiments, the in vitro life culture system 100 may include a culture module 110, a culture fluid provision module 120, and a fluid output module 130.

The culture module 110 may be used to cultivate a culture. The culture module 110 may include a culture chamber for holding a culture fluid. In some embodiments, the culture may be placed in the culture fluid of the culture chamber for cultivation. In some embodiments, the culture may include, but is not limited to, an organoid tissue cell, an organoid tumor cell, a microbial cell population, etc. In some embodiments, the culture may be cultivated in various manners such as a support culture, a suspension culture or a planar culture, etc. In some embodiments, the support culture may be confining the culture within a certain spatial region by a support structure (e.g., a substrate gel) for cultivation. In some embodiments, the suspension culture may be suspending the culture in the culture fluid for cultivation. In some embodiments, the planar culture may be attaching a culture to a culture plane for cultivation. In some embodiments, the culture fluid may be a fluid used for growth and maintenance of the culture (e.g., a cell population or a growth of a microorganism). In some embodiments, the culture fluid may include a nutrient component required for the growth of the culture. In some embodiments, the nutrient component may include, but is not limited to, at least one of a culture medium, a culture factor, a drug, an enzyme, a carbohydrate, a nitrogenous substance, an inorganic salt (including trace elements), and a vitamin. In some embodiments, the culture fluid may also include a gas (e.g., oxygen) required for the growth of the culture, which may be dissolved in the culture fluid. In some embodiments, a shape of the culture chamber may be arbitrary, including but not limited to rectangular, square, spherical, cylindrical, conical, irregular shape, etc.

The culture fluid provision module 120 may be used to provide a culture fluid to the culture chamber in the culture module 110. In some embodiments, the culture fluid provision module 120 may include a structure that directly or indirectly provides a culture fluid that meets growth requirements of the culture. In some embodiments, the culture fluid provision module 120 may include a container storing a required culture fluid, which is capable of feeding the stored culture fluid directly into the culture chamber. In some embodiments, the culture fluid provision module 120 may also include a mixing unit capable of configuring a culture fluid. In some embodiments, the mixing unit may be configured to mix oxygen with a nutrient component to form the culture fluid, and feed the culture fluid into the culture chamber. In some embodiments, the culture fluid provision module 120 may be provided inside the culture chamber or outside the culture chamber. In some embodiments, the culture fluid provision module 120, which is provided inside the culture chamber, may be provided with a corresponding opening or a corresponding pipeline for flowing the culture fluid into the inside of the culture chamber. In some embodiments, the culture fluid provision module 120, which is provided outside the culture chamber, may be connected with the culture chamber via a fluid flow pipeline and feed the culture fluid into the culture chamber via the fluid flow pipeline.

In some embodiments, the culture fluid provision module 120 may be used to provide a flowing culture fluid for the culture module 110 so that the culture fluid in the culture module 110 is in a continuous flowing state to provide the perfusion culture for the culture in the culture module 110. In this way, the culture is kept in a flowing culture fluid during the cultivation process, which can effectively increase a contact between the culture and the culture fluid and make the growth of the culture more adequate.

In some embodiments, the culture fluid in the culture module 110 may also not flow and the culture may be cultivated using a static culture after filling the culture module 110 with the culture fluid via the fluid provision module 120. In this way, an amount of the culture fluid consumed during the cultivation process may be effectively reduced, saving the cost of cultivation.

The fluid output module 130 may be used to discharge the culture fluid from the culture chamber. In some embodiments, the fluid output module 130 may include a structure for discharging the culture fluid. In some embodiments, the fluid output module 130 may be an opening or a pipeline provided in the culture chamber that may discharge the culture fluid in the culture chamber directly out of the in vitro life culture system 100. In some embodiments, the fluid output module 130 may collect the culture fluid to be discharged and determine whether the collected culture fluid is discharged out of the in vitro life culture system 100. In some embodiments, in response to a determination that a metabolite concentration of the collected culture fluid exceeds a preset threshold (e.g., 0.5 g/mL), the collected culture fluid is discharged out of the in vitro life culture system 100. In some embodiments, in response to a determination that the metabolite concentration of the collected culture fluid does not exceed the preset threshold (e.g., 0.5 g/mL), the collected culture fluid is recycled.

In some embodiments, the in vitro life culture system 100 may further include a control module 150 for controlling other modules of the in vitro life culture system 100 (e.g., the culture module 110, the culture fluid provision module 120, the fluid outlet module 130, etc.). In some embodiments, the control module 150 may control a culture temperature in the culture chamber of the culture module 110. In some embodiments, the control module 150 may control a flow rate of the culture fluid in the culture chamber of the culture module 110. In some embodiments, the control module 150 may control a rate at which the culture fluid is provided in the culture fluid provision module 120. In some embodiments, the control module 150 may control a discharge rate of the culture fluid in the culture fluid outlet module 130. In some embodiments, the control module 150 may include a processor with a data processing function. In some embodiments, the processor may obtain data generated by the modules or units in the in vitro life culture system 100 and process the data. In some embodiments, the processor may also generate a control instruction for the control module 150 to control other modules or units in the in vitro life culture system.

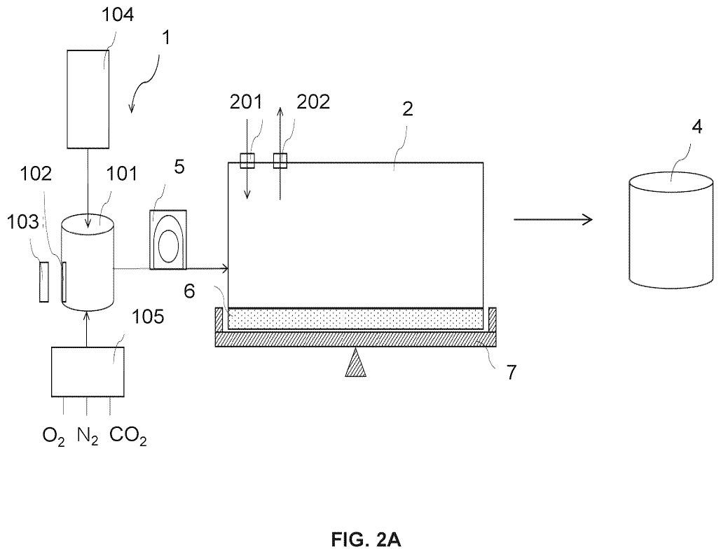

In some embodiments, referring to FIG. 2A, the culture fluid provision module 120 may include a mixing unit 1. The mixing unit 1 may be used to prepare a culture fluid.

In some embodiments, the mixing unit 1 may include a mixing chamber 101, which is used to mix a nutrient solution and a gas to form the culture fluid and deliver the culture fluid to the culture module 110. In some embodiments, the nutrient solution may be a fluid material that contains a nutrient component required for the growth of the culture. In some embodiments, the nutrient solution may include a nutrient substance such as a culture medium, a factor, a drug and an enzyme that is required for the growth of the culture. In some embodiments, the gas may be used to increase an amount of dissolved oxygen in the nutrient solution and adjust a potential of hydrogen (expressed as pH) of the nutrient solution. In some embodiments, the gas may include oxygen, carbon dioxide and an inert gas. The inert gas may include, but is not limited to, nitrogen, helium, neon, argon, xenon or radon gas. In some embodiments, the oxygen may be used to increase the amount of dissolved oxygen in the nutrient solution and other gases may be used to adjust the pH of the nutrient solution.

In some embodiments, the nutrient solution and the gas may be mixed to prepare a culture fluid. In some embodiments, the culture fluid may also be a formulated culture fluid prestored in a storage structure of the culture fluid provision module 120. In some embodiments, nutrient components required for culturing different cultures differ, and the culture fluid contains different components.

In some embodiments, the culture may be a liver tumor organoid cell, and the components of the culture fluid may include an additive, a serum substitute, Penicillin-Streptomycin (P/S), etc. In some embodiments, the additive may include, but is not limited to, an L-glutamine, a Glutamax™, etc. In some embodiments, the serum substitute may include, but is not limited to, N2, B27, etc.

In some embodiments, a concentration of a component in the culture fluid may be expressed as a dilution ratio of the component. In some embodiments, the dilution ratio may be a volume ratio of a dilution fluid (e.g., water) to a stock fluid of the component. In some embodiments, a concentration of the Glutamax™ in the culture fluid may be expressed as a dilution ratio of 100:3 to 100:0.1, i.e., adding 0.1 to 3 parts by volume of the Glutamax™ stock fluid for every 100 parts by volume of the dilution fluid in the culture fluid. In some embodiments, the concentration of the Glutamax™ in the culture fluid may be expressed as a dilution ratio of 100:2 to 100:0.1. In some embodiments, the concentration of the Glutamax™ in the culture fluid may be expressed as a dilution ratio of 100:3 to 100:0.5. In some embodiments, the concentration of the Glutamax™ in the culture fluid may be expressed as a dilution ratio of 100:1.5 to 100:0.5. In some embodiments, the concentration of the Glutamax™ in the culture fluid may be expressed as a dilution ratio of 100:1 to 100:0.5. In some embodiments, the concentration of the Glutamax™ in the culture fluid may be expressed as a dilution ratio of 100:1.5 to 100:1. In some embodiments, a concentration of the N2 in the culture fluid may be expressed as a dilution ratio of 100:3 to 100:0.1. In some embodiments, the concentration of the N2 in the culture fluid may be expressed as a dilution ratio of 100:2 to 100:0.1. In some embodiments, the concentration of the N2 in the culture fluid may be expressed as a dilution ratio of 100:3 to 100:0.5. In some embodiments, the concentration of the N2 in the culture fluid may be expressed as a dilution ratio of 100:1.5 to 100:0.5. In some embodiments, the concentration of the N2 in the culture fluid may be expressed as a dilution ratio of 100:1 to 100:0.5. In some embodiments, the concentration of the N2 in the culture fluid may be expressed as a dilution ratio of 100:1.5 to 100:1. In some embodiments, a concentration of the B27 in the culture fluid may be expressed as a dilution ratio of 50:3 to 50:0.1. In some embodiments, the concentration of the B27 in the culture fluid may be expressed as a dilution ratio of 50:2 to 50:0.1. In some embodiments, the concentration of the B27 in the culture fluid may be expressed as a dilution ratio of 50:3 to 50:0.5. In some embodiments, the concentration of the B27 in the culture fluid may be expressed as a dilution ratio of 50:1.5 to 50:0.5. In some embodiments, the concentration of the B27 in the culture fluid may be expressed as a dilution ratio of 50:1.5 to 50:0.5. In some embodiments, the concentration of the B27 in the culture fluid may be expressed as a dilution ratio of 50:1.5 to 50:1. In some embodiments, a concentration of the P/S in the culture fluid may be expressed as a dilution ratio of 100:3 to 100:0.1. In some embodiments, the concentration of the P/S in the culture fluid may be expressed as a dilution ratio of 100:2 to 100:0.1. In some embodiments, the concentration of the P/S in the culture fluid may be expressed as a dilution ratio of 100:3 to 100:0.5. In some embodiments, the concentration of the P/S in the culture fluid may be expressed as a dilution ratio of 100:1.5 to 100:0.5. In some embodiments, the concentration of the P/S in the culture fluid may be expressed as a dilution ratio of 100:1 to 100:0.5. In some embodiments, the concentration of the P/S in the culture fluid may be expressed as a dilution ratio of 100:1.5 to 100:1.

In some embodiments, the components of the culture fluid of the liver tumor organoid cell may also include an epidermal growth factor (EGF), a secreted protein (RSPO1), a stem cell growth factor (HGF), a fibroblast growth factor 10 (FGF10), an essential protein of the Wnt signaling pathway (Wnt3a), etc.

In some embodiments, the concentration of the component in the culture fluid may be expressed as a mass of the component contained in each milliliter of culture fluid (e.g., ng/mL, ng/mL). In some embodiments, a concentration of the EGF in the culture fluid may be in a range of 20 ng/mL to 80 ng/mL. In some embodiments, the concentration of the EGF in the culture fluid may be in a range of 30 ng/mL to 70 ng/mL. In some embodiments, the concentration of the EGF in the culture fluid may be in a range of 40 ng/mL to 60 ng/mL. In some embodiments, the concentration of the EGF in the culture fluid may be in a range of 45 ng/mL to 50 ng/mL. In some embodiments, the concentration of the EGF in the culture fluid may be in a range of 50 ng/mL to 55 ng/mL. In some embodiments, a concentration of the PSPO1 in the culture fluid may be in a range of 200 ng/mL to 800 ng/mL. In some embodiments, the concentration of the PSPO1 in the culture fluid may be in a range of 300 ng/mL to 700 ng/mL. In some embodiments, the concentration of the PSPO1 in the culture fluid may be in a range of 400 ng/mL to 600 ng/mL. In some embodiments, the concentration of the PSPO1 in the culture fluid may be in a range of 450 ng/mL to 500 ng/mL. In some embodiments, the concentration of the PSPO1 in the culture fluid may be in a range of 500 ng/mL to 550 ng/mL. In some embodiments, a concentration of the HGF in the culture fluid may be in a range of 5 ng/mL to 45 ng/mL. In some embodiments, the concentration of the HGF in the culture fluid may be in a range of 15 ng/mL to 35 ng/mL. In some embodiments, the concentration of the HGF in the culture fluid may be in a range of 20 ng/mL to 30 ng/mL. In some embodiments, the concentration of the HGF in the culture fluid may be in a range of 20 ng/mL to 25 ng/mL. In some embodiments, the concentration of the HGF in the culture fluid may be in a range of 25 ng/mL to 30 ng/mL. In some embodiments, a concentration of the FGF10 in the culture fluid may be in a range of 100 ng/mL to 300 ng/mL. In some embodiments, the concentration of the FGF10 in the culture fluid may be in a range of 150 ng/mL to 250 ng/mL. In some embodiments, the concentration of the FGF10 in the culture fluid may be in a range of 180 ng/mL to 220 ng/mL. In some embodiments, the concentration of the FGF10 in the culture fluid may be in a range of 180 ng/mL to 200 ng/mL. In some embodiments, the concentration of the FGF10 in the culture fluid may be in a range of 200 ng/mL to 220 ng/mL. In some embodiments, a concentration of the Wnt3a in the culture fluid may be in a range of 10 ng/mL to 50 ng/mL. In some embodiments, the concentration of the Wnt3a in the culture fluid may be in a range of 20 ng/mL to 40 ng/mL. In some embodiments, the concentration of the Wnt3a in the culture fluid may be in a range of 25 ng/mL to 35 ng/mL. In some embodiments, the concentration of the Wnt3a in the culture fluid may be in a range of 25 ng/mL to 30 ng/mL. In some embodiments, the concentration of the Wnt3a in the culture fluid may be in a range of 30 ng/mL to 35 ng/mL.

In some embodiments, the culture fluid of the liver tumor organoid cell may also include other nutrient components. In some embodiments, other nutrient components may include, but are not limited to, 4-(2-hydroxyethyl)piperazine-1-ethanesulfonic acid (HEPES), N-Acetyl-L-cysteine, gastrin, nicotinamide, small molecule compounds (A83.01), forskolin, etc.

In some embodiments, the culture may be a gastric organoid cell. In some embodiments, nutrient components of a culture fluid of the gastric organoid cell may include an additive, a serum substitute, penicillin-streptomycin (P/S). In some embodiments, the additive may include, but is not limited to, L-glutamine, Glutamax™, etc. In some embodiments, the serum substitute may include, but is not limited to, N2, B27, etc. In some embodiments, the concentrations of the Glutamax™, the N2, the B27 and the P/S in the culture fluid may be the same as that in the culture fluid for culturing the liver tumor organoid cell, which are not repeated herein.

In some embodiments, the nutrient components of the culture fluid of the gastric organoid cell may also include an EGF, a RSPO1, a recombinant protein (noggin), a recombinant human fibroblast growth factor 10 (hFGF10), etc.

In some embodiments, the concentrations of the EGF and the RSPO1 in the culture fluid may be the same as those in the culture fluid for culturing the liver tumor organoid cell, which are not repeated here. In some embodiments, a concentration of the hFGF10 in the culture fluid may be in a range of 100 ng/mL to 400 ng/mL. In some embodiments, the concentration of the hFGF10 in the culture fluid may be in a range of 150 ng/mL to 350 ng/mL. In some embodiments, the concentration of the hFGF10 in the culture fluid may be in a range of 150 ng/mL to 300 ng/mL. In some embodiments, the concentration of the hFGF10 in the culture fluid may be in a range of 150 ng/mL to 250 ng/mL. In some embodiments, the concentration of the hFGF10 in the culture fluid may be in a range of 200 ng/mL to 500 ng/mL. In some embodiments, a concentration of the noggin in the culture fluid may be in a range of 5 ng/mL to 45 ng/mL. In some embodiments, the concentration of the noggin in the culture fluid may be in a range of 15 ng/mL to 35 ng/mL. In some embodiments, the concentration of the noggin in the culture fluid may be in a range of 20 ng/mL to 50 ng/mL. In some embodiments, the concentration of the noggin in the culture fluid may be in a range of 20 ng/mL to 60 ng/mL. In some embodiments, the concentration of the noggin in the culture fluid may be in a range of 25 ng/mL to 50 ng/mL.

In some embodiments, the culture fluid of the gastric organoid cell may also include other nutrient components. In some embodiments, other nutrient components may include, but are not limited to, HEPES, N-acetyl-L-cysteine, nicotinamide, small molecule compounds (A83.01), selective ROCK1 inhibitors (Y27632), gastrin, Primocin, etc.

In some embodiments, the culture may be a colonic organoid cell. In some embodiments, nutrient components of the colonic organoid cell may include an additive, a serum substitute, a double resistant penicillin and a penicillin-streptomycin (P/S). In some embodiments, the additive may include, but is not limited to, L-glutamine, Glutamax™, etc. In some embodiments, the serum substitute may include, but is not limited to, N2, B27, etc. In some embodiments, concentrations of the Glutamax™ the N2, the B27 and the P/S in the culture fluid may be the same as those in the culture fluid for cultivating the liver tumor organoid cell, which are not repeated here.

In some embodiments, the nutrient components of the culture fluid of the colonic organoid may include an EGF, a secreted protein that activates Wnt signaling (RSPO1), a noggin, an essential protein of the Wnt signaling pathway (Wnt3a), etc. In some embodiments, concentrations of the components EGF, RSPO1, noggin and Wnt3a in the culture fluid of the colonic organoid may be the same as those in the culture fluid of the liver tumor organoid cell, which are not repeated here.

In some embodiments, the culture fluid of the colonic organoid may also include other nutrient components. In some embodiments, other nutrient components may include, but are not limited to, HEPES, N-acetyl-L-cysteine, nicotinamide, small molecule compounds (A83.01), selective p38 MAPK inhibitors (SB202190), selective ROCK1 inhibitor (Y27632), gastrin, hormone-like substance (Prostaglandine E2) and Primocin.

In some embodiments, the mixing unit 1 may also include a fluid replenishment chamber 104. In some embodiments, the fluid replenishment chamber 104 may be connected with the mixing chamber 101 for delivering a nutrient solution into the mixing chamber 101. In some embodiments, the fluid replenishment chamber 104 may also be used to store the nutrient solution. In some embodiments, the fluid replenishment chamber 104 may deliver a stored nutrient solution into the mixing chamber 101 according to an actual condition of the culture fluid in the mixing chamber 101. In some embodiments, the actual condition of the culture fluid in the mixing chamber 101 may be that during the delivery of the culture fluid from the mixing chamber 101 to the culture module 110, there may be too little culture fluid stored to meet the next delivery demand.

In some embodiments, a power unit 5 may be provided between the fluid replenishment chamber 104 and the mixing chamber 101. In some embodiments, the power unit may include a power source, a motor, etc. The power source may include battery and power supply system. In some embodiments, the power unit 5 may control a delivery amount and a delivery rate of the nutrient solution. The nutrient solution in the fluid replenishment chamber 104 may be delivered to the mixing chamber 101 in accordance with the usage requirements using the power unit 5.

In some embodiments, the mixing unit 1 may further include a gas mixing control unit 105 connected with the mixing chamber 101 for delivering a gas (e.g., oxygen, carbon dioxide, nitrogen, etc.) into the mixing chamber 101. In some embodiments, the gas mixing control unit may include a gas mixing controller.

In some embodiments, the mixing unit 1 may be configured to introduce the nutrient solution and the gas to the mixing chamber 101 via the fluid replenishment chamber 104 and the gas mixing control unit 105, and mix the nutrient solution and gas in the mixing chamber 101 to form the culture fluid. In some embodiments, the introduced oxygen may increase the amount of dissolved oxygen in the culture fluid to meet the growth requirements of the culture. In some embodiments, the amount of dissolved oxygen may be an amount of oxygen dissolved in the culture fluid. In some embodiments, the amount of dissolved oxygen in the culture fluid may be in a range of 0% to 100% of an air saturation of the culture fluid. In some embodiments, the amount of dissolved oxygen in the culture fluid may be in a range of 20% to 80% of the air saturation of the culture fluid. In some embodiments, the amount of dissolved oxygen in the culture fluid may be in a range of 20% to 60% of the air saturation of the culture fluid.

In some embodiments, the introduced carbon dioxide may regulate the pH of the culture fluid to meet the growth requirements of the culture.

In some embodiments, the introduced nitrogen may be used to dilute the culture fluid to reduce the amount of dissolved oxygen in the culture fluid. In some embodiments, the gas mixing control unit 105 may also be configured to introduce other inert gases to achieve the purpose of diluting the amount of dissolved oxygen in the culture fluid.

In some embodiments, a count of the mixing chamber 101 may be one and a count of the gas mixing control unit 105 may be one, and the gas mixing control unit may be connected with the mixing chamber. In some embodiments, a count of the mixing chamber 101 may be at least two, a count of the gas mixing control unit 105 may be one, and the gas mixing control unit may be connected with the at least two mixing chambers at the same time. In some embodiments, one gas mixing control unit may be capable of separately controlling the amount of gas introduced into the at least two mixing chambers. In some embodiments, one gas mixing control unit may separately control the amount of oxygen introduced into the at least two mixing chambers to further control the amount of dissolved oxygen in the culture fluid of the at least two mixing chambers. In some embodiments, one gas mixing control unit may separately control the amount of carbon dioxide introduced into the at least two mixing chambers to control a carbon dioxide concentration of the culture fluid in the at least two mixing chambers, and further control the pH of the culture fluid. In some embodiments, one gas mixing control unit may separately control the amount of nitrogen gas introduced into the at least two mixing chambers to achieve a purpose of diluting the amount of dissolved oxygen in the culture fluid of each mixing chamber.

In some embodiments, the mixing unit 1 may also include a pH detection member 102, a dissolved oxygen detection member 103, and a signal detector. In some embodiments, the pH detection member 102 and the dissolved oxygen detection member 103 may be provided in the mixing chamber 101. In some embodiments, the signal detector may be provided inside or outside the mixing chamber 101. The signal detector may be capable of sensing signals from the pH detection member 102 and the dissolved oxygen detection member 103 to obtain a pH and an amount of dissolved oxygen of the culture fluid in the mixing chamber 101.

In some embodiments, as the pH and the amount of dissolved oxygen in the mixing chamber 101 change, the signals detected by the pH detection member 102 and the dissolved oxygen detection member 103 change. In some embodiments, the pH detection member 102 and the dissolved oxygen detection member 103 may include an optical detection assembly that generates an optical signal. In some embodiments, as the pH and the amount of dissolved oxygen of the culture fluid in the mixing chamber change, the optical signals of the pH detection member 102 and the dissolved oxygen detection member 103 change, the signal detector may periodically receives the optical signals on surfaces of the pH detection member 102 and the dissolved oxygen detection member 103 to obtain data feedback, and the pH and the amount of dissolved oxygen may be calculated according to the data feedback. In some embodiments, the pH detection member 102 and the dissolved oxygen detection member 103 may include an electrical detection assembly that generates an electrical signal.

In some embodiments, the pH detection member 102 may include a pH electrode piece and the dissolved oxygen detection member 103 may include a dissolved oxygen electrode piece. The pH electrode piece may be provided on an inner wall of the mixing chamber 101 or in the mixing chamber 101 and the dissolved oxygen detection member 103 may be provided on the inner wall of the mixing chamber 101 or immersed in the culture fluid of the mixing chamber 101. In some embodiments, as the pH and the amount of dissolved oxygen in the culture fluid of the mixing chamber change, electrical signals from the pH electrode piece and the dissolved oxygen electrode piece change. The signal detector may periodically receive the electrical signals from the pH electrode piece and the dissolved oxygen electrode piece to obtain data feedback, and the pH and the amount of dissolved oxygen of the culture fluid may be calculated based on the feedback data.

In some embodiments, the signal detector may feed detected information back to the processor so that the processor may generate a control instruction based on the detected information to control the fluid replenishment chamber 104 and the gas mixing control unit 105 to adjust the amount of dissolved oxygen and the pH of the culture fluid. In some embodiments, if the amount of dissolved oxygen of the culture fluid is too high, the processor may cause the fluid replenishment chamber 104 to deliver more nutrient solution to the mixing chamber 101 for dilution to reduce the amount of dissolved oxygen of the culture fluid in the mixing chamber 101, and if the amount of dissolved oxygen is too low, the processor may cause the gas mixing control unit 105 to introduce the oxygen into the mixing chamber 101 to increase the amount of dissolved oxygen of the culture fluid in the mixing chamber 101. If the pH is above a first threshold, the processor may control the gas mixing control unit 105 to introduce the carbon dioxide to lower the pH of the culture fluid in the mixing chamber 101; and if the pH is below a second threshold, the processor may control the fluid replenishment chamber 104 to deliver more nutrient solution to the mixing chamber 101 for dilution or introduce alkaline solution into the mixing chamber 101 to raise the pH of the culture fluid in the mixing chamber 101. In some embodiments, the first threshold may be in a range of 7.6 to 8. In some embodiments, the first threshold may be 7.6, 7.8, or 8. In some embodiments, the second threshold may be in a range of 6.6 to 7. In some embodiments, the second threshold may be 6.6, 6.8, or 7.

In some embodiments, the mixing chamber 101 may be also provided with an air outlet, which may discharge the gas in the mixing chamber 101 to ensure the stability of the air pressure in the mixing chamber 101. In some embodiments, the mixing chamber 101 may be provided with a stirring assembly, and the stirring assembly may stir the fluid in the mixing chamber 101 to mix evenly and make the gas sufficiently enter into the fluid. In some embodiments, the mixing chamber 101 may be further provided with an alkali addition opening, and alkali solution may be added to the mixing chamber 101 through the alkali addition opening to adjust the pH of the culture fluid in the mixing chamber 101.

In some embodiments, the nutrient solution may be mixed with the oxygen or the like in the mixing chamber 101 to form the culture fluid, and by controlling corresponding variables (e.g., the pH and the amount of dissolved oxygen), the culture fluid formed by mixing the nutrient solution with the oxygen or the like may be more suitable for the growth of the culture, which may adequately provide the nutrient required for the growth of the culture. Compared to directly feeding the culture chamber with the nutrient solution and the oxygen, etc., it saves the design of complicated pipelines, valves and probes, and reduces the system cost.

In some embodiments, the culture fluid provision module 120 may also not include the mixing unit 1, but may include a storage structure storing the formulated culture fluid. The storage structure may be used to introduce the formulated culture fluid directly into the culture chamber 2 for use in cultivating the culture. In some embodiments, the components of the culture fluid and the concentrations of the components can be found in the descriptions elsewhere in the present disclosure and are not repeated herein.

In some embodiments, the culture module 110 may include a culture unit. In some embodiments, the culture unit may include a culture chamber 2, the culture chamber 2 being used to hold the culture fluid and the culture. In some embodiments, the culture chamber 2 may be connected with the mixing chamber 101, and the culture fluid in the mixing chamber 101 may be delivered into the culture chamber 2. In some embodiments, a culture fluid delivery pipeline may be provided between the mixing chamber 101 and the culture chamber 2. In some embodiments, the culture fluid delivery pipeline may be a hose made of airtight material. In some embodiments, the airtight material may be obtained by applying an airtight coating to a polymeric material. In some embodiments, the airtight coating may be a multilayer polymer coating, each of which has different molecular orientation. In some embodiments, the airtight coating may also be a multilayer laminated film prepared with a clay material as the primary material and a resin material as an additive. The above airtight coating also has a strong adhesive property and is not easily detached from the hose when the shape of the hose changes. According to a relative setting position of the mixing chamber 101 and the culture chamber 2 and the need for a delivery rate and a delivery amount of the culture fluid, it may be determined whether a power unit 5 is provided between the mixing chamber 101 and the culture chamber 2 for delivering the culture fluid in the mixing chamber 101 to the culture chamber 2. In some embodiments, in order to prevent a possible condition of insufficient airtightness of the culture fluid delivery pipeline (e.g., wear and tear of the airtight coating), the culture fluid delivery pipeline may be placed in a sealed environment, which may be filled with a gas having a same component and a same concentration as those of the gas provided by the gas mixing control unit 105, so as to cause the concentration of the gas both inside and outside of the culture fluid delivery pipeline to a balance, so that the gas in the culture fluid inside the culture fluid delivery pipeline does not escape through the culture fluid delivery pipeline.

In some embodiments, the perfusion culture of the culture in the culture chamber 2 may be achieved by feeding the culture fluid into the culture chamber 2 through the mixing chamber 101. In some embodiments, in order to prevent a condition where the culture is not adapted to the perfusion culture at the beginning of the cultivating culture, the culture in the culture chamber 2 may be subjected to a static culture for a first preset time, and when the first preset time is ended, the culture may be subjected to the perfusion culture. In some embodiments, the first preset time may be set according to a type of culture. In some embodiments, the culture may be an organoid cell, and the first preset time may be in a range of 1 hour to 72 hours. In some embodiments, the gas mixing control unit 105 may be connected with the culture chamber 2, and the oxygen may be supplemented to the culture fluid in the culture chamber 2 or the pH of the culture fluid may be adjusted through the gas mixing control unit 105 during the static culture of the culture.

In some embodiments, referring to FIG. 2B, the gas mixing control unit 105 may be connected with a cavity above the culture fluid in the mixing chamber 101, which may also be connected with the culture chamber 2 at the same time, and the gas may flow through the cavity and then into the culture chamber 2. In some embodiments, a gas delivery pipeline 1011 may be provided between the cavity above the culture fluid in the mixing chamber 101 and the culture chamber 2. In some embodiments, the gas delivery pipeline may be fitted with a valve, the valve being used to open or close the gas delivery pipeline 1011. In some embodiments, a culture fluid delivery pipeline 1012 may also be provided between the mixing chamber 101 and the culture chamber 2. In some embodiments, the gas delivery pipeline 1011 and the culture fluid delivery pipeline 1012 may be connected with the culture chamber 2 through a same interface. In some embodiments, a three-way valve may be provided among the gas delivery pipeline 1011, the culture fluid delivery pipeline 1012, and the interfaces of the culture chamber 2, through which either the gas delivery pipeline 1011 or the culture fluid delivery pipeline 1012 may be controlled to be connected with the culture chamber 2. In some embodiments, the power unit 5 may be provided between the gas delivery pipeline 1011 and the culture chamber 2. In some embodiments, the power unit 5 may also be provided between the culture liquid delivery pipeline 1012 and the culture chamber 2. In some embodiments, the power unit 5 may also be provided at an interface of the culture chamber 2, such that the power unit 5 may control a rate of the gas or culture fluid delivered to the culture chamber 2 in the gas delivery pipeline 1011 or the culture fluid delivery pipeline 1012.

In some embodiments, the gas mixing control unit 105 may also not be connected with the mixing chamber 101 but directly to the culture chamber 2, i.e., the gas in the gas mixing control unit 105 may be directly introduced into the culture chamber 2. In some embodiments, the in vitro life culture system 100 may also include a plurality of gas mixing control units 105, and each of the culture chambers and each of the mixing chambers in the in vitro life culture system 100 may be connected with one gas mixing control unit.

In some embodiments, a count of the culture chamber 2 may be one, i.e., one mixing chamber corresponds to one culture chamber. In some embodiments, a count of the culture chamber may be a plurality, and the plurality of culture chambers may be all connected with the mixing chamber 101. In some embodiments, the plurality of culture chambers may be connected in parallel, each of the culture chambers may be connected with one mixing chamber, all of the culture chambers 2 may be connected with one mixing chamber, or several culture chambers may be connected with together to one mixing chamber. In some embodiments, the plurality of culture chambers may also be connected in series, the culture fluid flowing sequentially through the plurality of culture chambers. In some embodiments, a plurality of cultures may be cultivated at the same time by providing the plurality of culture chambers, which is convenient for ensuring the consistency of the cultures during a subsequent test, thereby ensuring the accuracy of the comparison of test results.

In some embodiments, the culture chamber 2 may be provided with a sample addition opening 201 and a sample taking opening 202. The culture may be added to the culture chamber 2 through the sample addition opening 201. A growth factor, a drug, or the like may also be added to the culture chamber 2 through the sample addition opening 201 to promote or inhibit the growth of the culture during the cultivation process according to the culture requirements, and an enzyme, a matrix gel treatment reagent, a staining reagent, or the like may also be added to the system at end of the cultivation for collecting, treating, or characterizing the culture. a culture sample or a culture fluid sample may be taken during the cultivation process through the sample taking opening 202 for analysis.

During the cultivation process, in order to control a temperature of the culture fluid in the culture chamber 2 to perform different operations on the culture at different temperatures, in some embodiments, the culture module 110 may further include a temperature control unit 6. In some embodiments, the temperature control unit 6 may be configured to control the temperature of the culture fluid to be at a physiological temperature of the culture to cultivate the culture. In some embodiments, the temperature control unit 6 may be configured to control the temperature of the culture fluid to be at a liquefaction temperature of a support structure of the culture so that the culture may be removed at the end of the cultivation. For more information about the temperature control unit 6, please refer to the elsewhere in the present disclosure.

In order to obtain real-time information about the growth of the culture during the cultivation of the culture, in some embodiments, the culture module 110 may further include a microscopic observation module 8. In some embodiments, a culture personnel may observe the culture in the culture chamber 2 via the microscopic observation module 8. In some embodiments, the microscopic observation module 8 may be configured to obtain image data of the culture for determining the growth of the culture, and further, the culture personnel or the in vitro life culture system 100 may make an adjustment to the cultivation of the culture. For more information about the microscopic observation module 8, please refer to elsewhere in the present disclosure.

In some embodiments, the fluid output module 130 may include a collection unit 4. In some embodiments, the collection unit may include a collection container. In some embodiments, the collection unit 4 may be connected with the culture chamber 2 for discharging and collecting the culture fluid in the culture chamber 2.

In some embodiments, the fluid output module 130 may also not include the collection unit 4, but may include a waste fluid outlet provided in the culture chamber 2, and the waste fluid outlet may be used for discharging the culture fluid of the culture chamber 2 out of the in vitro life culture system 100. In some embodiments, the waste fluid outlet may be an arbitrarily shaped structure that may guide flow, and the shape and structure of the waste fluid outlet are not limited in the present disclosure.

In some embodiments, through a cooperation of the culture fluid provision module 120, the culture module 110, and the fluid outlet module, the culture (e.g., an organoid cell, etc.) may be placed in a dynamic perfusion cultivation environment, which ensures a fast growth rate of the culture and a short cultivation cycle, thereby ensuring the extent of the growth of the culture during the cultivation cycle.

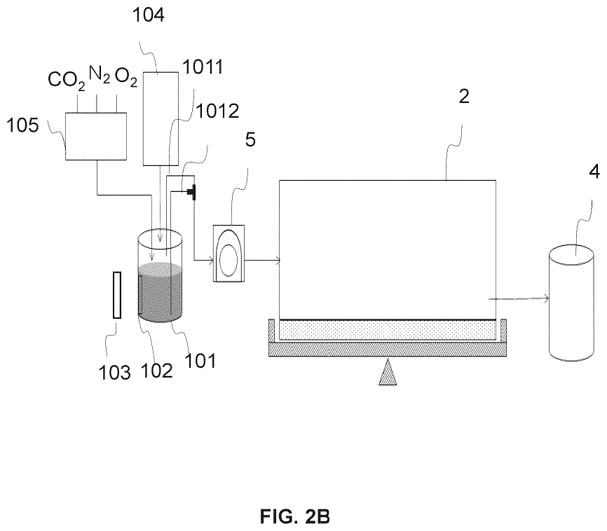

In some embodiments, the culture fluid in the culture chamber 2 may be discharged through the fluid outlet module after use, resulting in a low utilization rate of the culture fluid and a high cultivation cost. In some embodiments, referring to FIG. 3, the in vitro life culture system 100 may further include a culture fluid circulation module 140. In some embodiments, the culture fluid circulation module 140 may be used to achieve a recirculation of the culture fluid in the culture chamber 2 to increase the utilization rate of the culture fluid and reduce the waste of the culture fluid.

Since the culture fluid is mixed with metabolites produced by the culture after the culture is cultivated in culture fluid of the culture unit, a metabolite concentration needs to be controlled within a preset range before the culture fluid is circulated for use. In some embodiments, the culture fluid circulation module 140 may include a component exchange structure for performing a component exchange of metabolites in the culture fluid. In some embodiments, the culture fluid circulation module 140 may also include a structure for controlling the circulation of the fluid, which is used to circulate the culture fluid. In some embodiments, the culture fluid circulation module 140 may be provided inside or outside the culture chamber 2.

In some embodiments, the culture fluid circulation module 140 provided inside the culture chamber 2 may include a component exchange structure. The component exchange structure may perform a component exchange of the culture fluid to be discharged in the culture chamber 2, and guide the recyclable culture fluid obtained after the component exchange into the culture fluid provision module 120 or the culture module 110 for reuse. In some embodiments, the component exchange may be a flow of a portion of a component of a culture fluid in different chambers from one chamber to another chamber. In some embodiments, the components of the culture fluid that are subject to the component exchange may include a nutrient component and a metabolite, i.e., the nutrient component in the first chamber flows into the second chamber, and the metabolite in the second chamber enters the first chamber as an exchange. In some embodiments, the component exchange structure may include, but is not limited to, a permeable membrane or a unidirectional component exchange membrane, etc. In some embodiments, the culture fluid circulation module 140 provided outside the culture chamber 2 may also include a component exchange structure. In some embodiments, the culture fluid circulation module 140 provided outside the culture chamber 2 may receive a culture fluid flowing out of the culture chamber 2, and after the component exchange of the received culture fluid, guide the obtained recyclable culture fluid to the culture fluid provision module 120 or the culture module 110 for reuse.

Referring to FIGS. 3-6, in some embodiments, the culture fluid circulation module 140 may include a first exchange unit 3 disposed outside the culture chamber 2. In some embodiments, the first exchange unit 3 may be connected with the culture chamber 2, and the first exchange unit 3 receives the culture fluid flowing out of the culture chamber 2 and performs the component exchange of the culture fluid. In some embodiments, the components of the culture fluid flowing out of the culture chamber 2 may include a nutrient component and a metabolite, and after the culture fluid flows into the first exchange unit 3, the nutrient component and the metabolite of the culture fluid in different chambers may be exchanged between different chambers of the first exchange unit 3, so that the concentrations of the metabolite and the nutrient component of the culture fluid in the different chambers of the first exchange unit 3 are changed. In some embodiments, the first exchange unit 3 may include a plurality of exchange chambers and membrane assemblies disposed among the plurality of exchange chambers. The exchange chamber may be used to receive and hold the culture fluid flowing out of the culture chamber 2. In some embodiments, the membrane assembly may be used to perform a component exchange of components in the culture fluid for filtering out non-reusable and reusable substances from components of the culture fluid, making the first exchange unit 3 discharge the non-reusable substance and deliver the reusable substance to the culture chamber 2. In some embodiments, the reusable substance may include a nutrient component. In some embodiments, the non-reusable substance may include a metabolite.

In some embodiments, the first exchange unit 3 may be provided to make the in vitro life culture system 100 achieve the perfusion cultivation while retaining the useful components of the culture fluid through the plurality of exchange chambers and the membrane assemblies and refluxing them into the culture chamber 2 for re-absorption and utilization, which improves the utilization rate of the culture fluid and reduces the cultivation cost.

In some embodiments, the first exchange unit 3 may be provided outside the culture chamber 2, so that an effective area of the membrane assembly may be set larger and the structure may be more flexible, which in turn improves the exchange efficiency.

In some embodiments, the first exchange unit 3 may be provided inside the culture chamber 2, so that the in vitro life culture system 100 does not need to additionally add a new structure, making the structure of the in vitro life culture system 100 simpler and less cost.

In some embodiments, an exchange chamber of the first exchange unit 3 may include a first exchange chamber 302 and a second exchange chamber 303, a membrane assembly 301 may be provided between the first exchange chamber 302 and the second exchange chamber 303, and the membrane assembly 301 may be used to retain or permeate at least a portion of the components in the culture fluid. In some embodiments, the first exchange chamber 302 may be capable of receiving the culture fluid flowing out of the culture chamber 2, the first exchange chamber 302 may be connected with the second exchange chamber 303 by the membrane assembly 301, and the metabolite in the culture fluid may be exchanged to the second exchange chamber 303, so that the metabolite concentration in the culture fluid may be reduced. In some embodiments, after the metabolite concentration of the culture fluid in the first exchange chamber 302 is reduced, the first exchange chamber 302 may be capable of refluxing the culture fluid to the culture chamber 2 or the mixing unit 1. In some embodiments, the membrane assembly 301 may also retain nutrient components required by the culture in the culture fluid in the first exchange chamber 302, such as, a serum, a growth factor, and an enzyme, etc., and metabolites produced by the growth of the culture in the first exchange chamber 302, such as, urea, carbon dioxide, etc., may be permeated into the second exchange chamber 303 through the membrane assembly 301, and then be discharged from the second exchange chamber 303.

In some embodiments, the first exchange chamber 302 may include a first interface and a second interface, the first interface may be used to receive the culture fluid flowing into the first exchange chamber 302, and the second interface may be used to flow out the culture fluid from the first exchange chamber 302. In some embodiments, the first interface may be connected with the culture chamber 2 to receive the culture fluid from the culture chamber 2. In some embodiments, the second interface may be connected with the culture chamber 2 to reflux the culture fluid to the culture chamber 2. In some embodiments, the second interface may be connected with one of the chambers in the culture fluid provision module 120 to reflux the culture fluid into the culture fluid provision module 120. In some embodiments, the second interface may be connected with the mixing chamber 101 to reflux the culture fluid into the mixing chamber 101. In some embodiments, the second interface may also be connected with the fluid replenishment chamber 104 to reflux the culture fluid into the fluid replenishment chamber 104.

In some embodiments, a metabolite concentration detection unit may be provided in the first exchange chamber 302 for detecting a metabolite concentration of the culture fluid in the first exchange chamber 302. In some embodiments, the metabolite concentration detection unit may include a concentration detector. In some embodiments, the metabolite concentration may be used to determine a percentage of the metabolite in the culture fluid and determine whether to continue filter the metabolite in the culture fluid. In some embodiments, when the metabolite concentration of the culture fluid is below a preset threshold (e.g., 2%; 3%; 5%, etc.), the culture fluid may be circulated.

In some embodiments, the metabolite concentration detection unit may use a sample detection or an in-situ detection to detect. In some embodiments, the in-situ detection may be a detection performed on the substance or component to be detected (e.g., a metabolite) at its original position. In some embodiments, the in-situ detection may include, but is not limited to, an infrared spectroscopy technique, a fluorescence detection technique, etc. In some embodiments, the fluorescence detection technique may include labelling a portion of the substance or component to be detected (e.g., a metabolite) using a fluorescent dye, irradiating the labelled substance by an excitation beam (e.g., a laser) to cause the substance to emit fluorescence, and then capturing the fluorescent beam for concentration detection. In some embodiments, the infrared spectroscopy technique may include irradiating the culture fluid to be detected with an infrared light, and measuring the absorption of the culture fluid at characteristic peak wavelength in the spectrum of the infrared light to perform the concentration detection.

In some embodiments, the second interface may be a selectively connectable interface with a function of opening or closing the interface. In some embodiments, the first exchange unit 3 may open or close the second interface based on a detection result of the metabolite concentration detection unit. In some embodiments, the detection result of the metabolite concentration detection unit may be that a metabolite concentration of the culture fluid is below the preset threshold (e.g., 2%; 3%; 5%, etc.), which indicates that the metabolite concentration of the culture fluid does not affect the reuse of the culture fluid. In some embodiments, when the detection result of the metabolite concentration detection unit is that a metabolite concentration of the culture fluid is lower than the preset threshold, it is indicated that the culture fluid in the first exchange chamber 302 may be reused, and the first exchange unit 3 is configured to open the second interface to make the culture fluid be guided into the culture fluid provision module 120 or the culture chamber 2. In other embodiments, when the detection result of the metabolite concentration detection unit is that a metabolite concentration of the culture fluid is higher than the preset threshold, it is indicated that the metabolite concentration of the culture fluid in the first exchange chamber 302 is too high to be reused, and the first exchange unit 3 may be configured to close the second interface to make the culture fluid continue to be exchanged for components in the first exchange chamber 302.

In some embodiments, the second exchange chamber 303 may include a third interface for discharging the culture fluid from the second exchange chamber 303. In some embodiments, the third interface may be connected with the collection unit 4 such that the collection unit 4 may collect the culture fluid in the second exchange chamber 303. In some embodiments, the third interface may also not be connected with the collection unit 4, but directly discharge the culture fluid in the second exchange chamber 303 out of the in vitro life culture system 100.

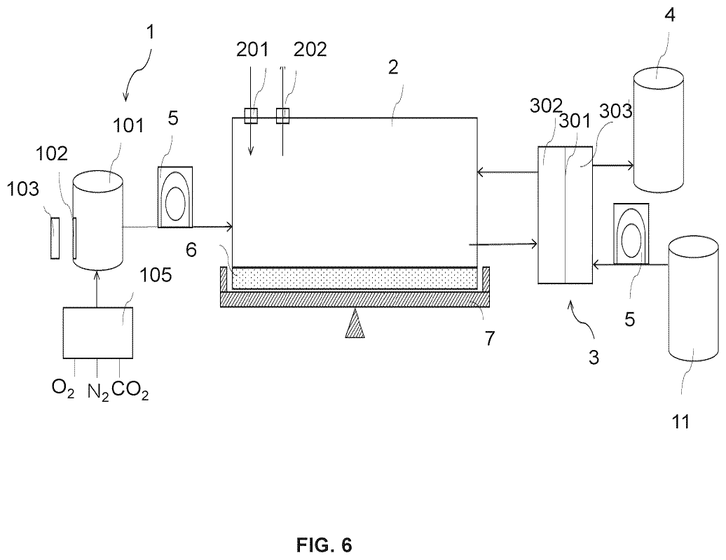

In some embodiments, referring to FIG. 7, an exchange chamber of the first exchange unit 3 may include a first exchange chamber 302, a second exchange chamber 303, and a third exchange chamber 304. In some embodiments, the first exchange chamber 302 may be connected with the second exchange chamber 303 and the third exchange chamber 304, respectively. A first unidirectional membrane assembly 3011 may be provided between the first exchange chamber 302 and the second exchange chamber 303, and the first unidirectional membrane assembly 3011 may be used for permeating the nutrient component in the culture fluid and retaining the metabolite. A second unidirectional membrane assembly 3012 may be provided between the first exchange chamber 302 and the third exchange chamber 304, and the second unidirectional membrane assembly 3012 may be used for permeating the metabolite in the culture fluid and retaining the nutrient component.

In some embodiments, the first exchange chamber 302 may be capable of receiving the culture fluid flowing out of the culture chamber 2, which is connected with the second exchange chamber 303 via the first unidirectional membrane assembly 3011, so that the culture fluid in the second exchange chamber 303 does not contain the metabolite. The metabolite may permeate into the third exchange chamber 304 through the second unidirectional membrane assembly 3012 and may be further discharged out of the in vitro life system 100 through the collection unit 4 connected with the third exchange chamber 304.

In some embodiments, the first exchange chamber 302 may include a first interface, and the first interface may be used to receive the culture fluid flowing into the first exchange chamber 302. In some embodiments, the first interface may be connected with the culture chamber 2 to receive the culture fluid flowing out from the culture chamber 2.

In some embodiments, the second exchange chamber 303 may include a second interface, the second interface may be connected with the culture chamber 2 to deliver the reusable culture fluid not containing or containing a small amount of the metabolite to the culture chamber 2. In some embodiments, the second interface of the second exchange chamber 303 may be connected with one of the chambers in the culture fluid provision module 120 to deliver the culture fluid to the culture fluid provision module 120. In some embodiments, the second interface may be connected with the mixing chamber 101 to deliver the culture fluid into the mixing chamber 101. In some embodiments, the second interface may also be connected with the fluid replenishment chamber 104 to deliver the culture fluid into the fluid replenishment chamber 104.

In some embodiments, the third exchange chamber 304 may include a third interface, and the third interface may be connected with the outside of the system for discharging the metabolite to the outside of the system. In some embodiments, the third interface may be connected with the collection unit 4 to discharge the metabolite into the collection unit for collection.

In some embodiments, the count of the culture chamber 2 may be one, the count of the first exchange unit 3 may also be one, and the one first exchange unit may be connected with the one culture chamber.

In some embodiments, the count of the culture chamber 2 may exceed 1, and the plurality of chambers may be connected in parallel. In some embodiments, the count of the first exchange unit 3 may be one, and the plurality of culture chambers connected in parallel may be connected with the one first exchange unit at the same time. In some embodiments, the count of the first exchange unit 3 may exceed 1, and each of the plurality of culture chambers connected in parallel may be connected with the one exchange unit.

In some embodiments, the count of the culture chamber 2 may exceed 1, and the plurality of chambers may be connected in series. In some embodiments, the count of the first exchange unit 3 may be one, and the first exchange unit may be connected with one (e.g., the last) of the plurality of culture chambers connected in series. When the plurality of culture chambers are connected in series, the “front” of the series connection refers to a portion of the culture chambers where the culture fluid flows first, and the “rear” of the series connection refers to a portion of the culture chambers where the culture fluid flows later. In some embodiments, the count of the first exchange unit 3 may exceed 1, and each of the plurality of culture chambers connected in series may be connected with the one first exchange unit located after the culture chamber. In some embodiments, each of the plurality of culture chambers connected in series may be connected with the one first exchange unit located after that culture chamber via the first interface to flow the culture fluid into the first exchange chamber 302, and the second interface of the first exchange chamber 302 may be connected with a later culture chamber connected in series to flow the culture fluid in the first exchange chamber 302 into the later culture chamber connected in series for cultivation of the culture. In some embodiments, the metabolite produced in each of the culture chambers may be discharged through the second exchange chamber 303 of the first exchange unit 3 behind the culture chamber.

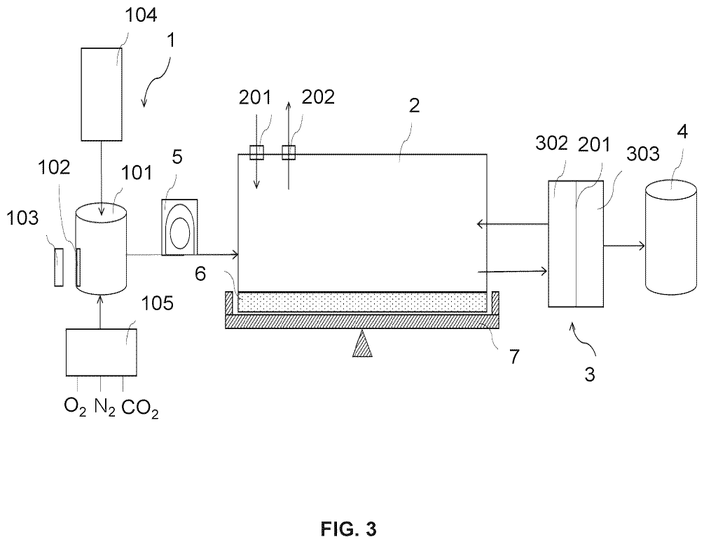

Referring to FIG. 4, in some embodiments, the culture fluid circulation module 140 may further include a power unit 5, the power unit 5 being used to control a flow rate of the culture fluid in the first exchange chamber 302 or the second exchange chamber 303. In some embodiments, the power unit 5 may be provided inside or outside the exchange unit to control the flow rate of the culture fluid of the first exchange chamber 302 or the second exchange chamber 303 in the exchange unit. In some embodiments, the power unit 5 may also be provided inside any one chamber (e.g., the mixing chamber 101, the culture chamber 2, etc.) containing the culture fluid to achieve the control of the flow rate of the culture fluid inside the chamber. In some embodiments, the power unit 5 may also be provided between any two connected chambers to control the flow rate of the culture fluid.

In some embodiments, any one or more interfaces (e.g., the first interface, the second interface, or the third interface) of the first exchange unit 3 may be provided with the power unit 5. In some embodiments, the power unit 5 may be provided between the culture chamber 2 and the first interface to deliver the culture fluid in the culture chamber 2 to the first exchange chamber 302 via the first interface. In some embodiments, the power unit 5 may also be provided between the culture chamber 2 and the second interface to deliver the culture fluid in the first exchange chamber 302 to the culture chamber 2 via the second interface.

In some embodiments, when the first exchange chamber 302 and the mixing chamber 101 are connected, the power unit 5 may also be provided between the first exchange chamber 302 and the mixing chamber 101, and the power unit 5 may be used to deliver the culture fluid in the first exchange chamber 302 into the mixing chamber 101. In some embodiments, the power unit 5 may be provided at the second interface to control the switch and flow rate of the fluid flowing the from the first exchange chamber 302 to the mixing chamber 101 by the second interface.

In some embodiments, the exchange unit 5 may be controlled by the control module 150. In some embodiments, the control module 150 may control the switch of the first interface, the second interface, or the third interface through controlling the exchange unit 5. In some embodiments, the control module 150 may also control the flow rate of the culture fluid at each interface (the first interface, the second interface, or the third interface) through the exchange unit 5.

In some embodiments, the flow rate of the culture fluid in the first exchange chamber 302 may be different from the flow rate of the culture fluid in the second exchange chamber 303. In some embodiments, the flow rate of the culture fluid in the first exchange chamber 302 may be lower than the flow rate of the culture fluid in the second exchange chamber 303. In some embodiments, the metabolite concentration of the culture fluid in the first exchange chamber 302 may be higher than the metabolite concentration of the culture fluid in the second exchange chamber 303, and the metabolite may permeate to the second exchange chamber 303 through the membrane assembly 301, and when the flow rate of the culture fluid in the second exchange chamber 303 is higher than the flow rate of the culture fluid in the first exchange chamber 302, the metabolite permeation may be effectively promoted to improve the exchange efficiency of the metabolite between the first exchange chamber 302 and the second exchange chamber 303. In some embodiments, the flow rate of the culture fluid in the first exchange chamber 302 may be higher than the flow rate of the culture fluid in the second exchange chamber 303. In some embodiments, a concentration of the nutrient component of the culture fluid in the second exchange chamber 303 may be higher than a concentration of the nutrient component of the culture fluid in the first exchange chamber 302, and the nutrient component may permeate to the first exchange chamber 302 through the membrane assembly 301, and when the flow rate of the culture fluid in the first exchange chamber 302 is higher than the flow rate of the culture fluid in the second exchange chamber 303, the permeation effect of the nutrient component may be effectively promoted to improve the exchange efficiency of the nutrient component between the first exchange chamber 302 and the second exchange chamber 303.

In some embodiments, the flow rate of the culture fluid in the first exchange chamber 302 and the second exchange chamber 303 may also not be controlled by the power unit 5, but may be controlled by a pore size of a permeable pore of the membrane assembly 301. In some embodiments, the membrane assembly 301 having different pore size may make flow rate of the culture fluid in the first exchange chamber 302 and the second exchange chamber 303 different. In some embodiments, the larger the pore size of the membrane assembly 301, the larger the permeation rate of the culture fluid between the first exchange chamber 302 and the second exchange chamber 303, and the larger the flow rate of the culture fluid in the first exchange chamber 302 and the second exchange chamber 303. The smaller the pore size of the membrane assembly 301, the smaller the permeation rate of the culture fluid between the first exchange chamber 302 and the second exchange chamber 303, and the smaller the flow rate of the culture fluid in the first exchange chamber 302 and the second exchange chamber 303.

In some embodiments, the culture fluid circulation module 140 may further include a fluid replenishment unit 11. In some embodiments, the fluid replenishment unit may include a fluid replenishment container. The fluid replenishment unit 11 may be used to deliver one or more components required for the culture to other chambers. In some embodiments, the fluid replenishment unit 11 may be connected with any chamber in the first exchange unit 3 (e.g., the first exchange chamber 302, the second exchange chamber 303, or the third exchange chamber 304) for delivering one or more components required for the culture to any chamber of the first exchange unit 3. In some embodiments, nutrient components of the culture fluid often does not directly meet the concentration requirements for reuse due to permeation of the culture fluid in the various chambers of the first exchange unit 3 (e.g., the first exchange chamber 302, the second exchange chamber 303, or the third exchange chamber 304), and therefore, it is necessary to supplement nutrient components to the culture fluid in these chambers so that the nutrient components of the culture fluid may be reused. In some embodiments, the control module 150 may determine whether to deliver one or more components required for the culture to the various chambers via the fluid replenishment unit 11 based on the amount of the nutrient components or metabolite of the culture fluid in the various chambers of the first exchange unit 3.

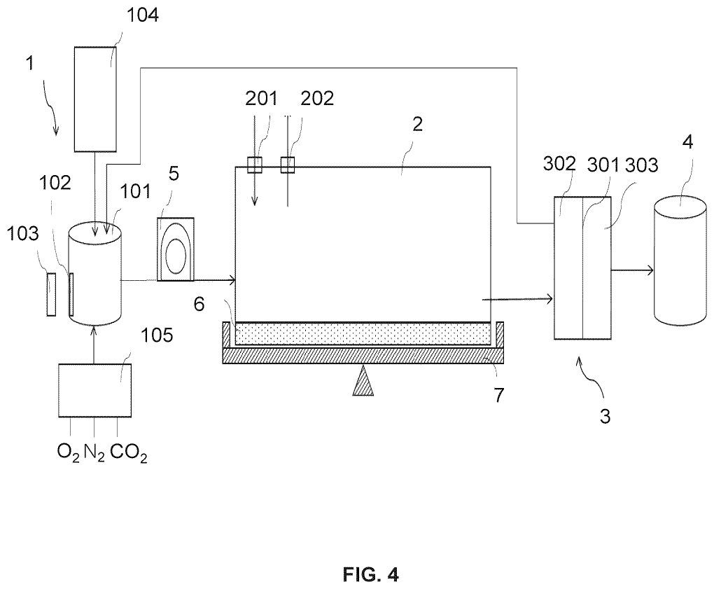

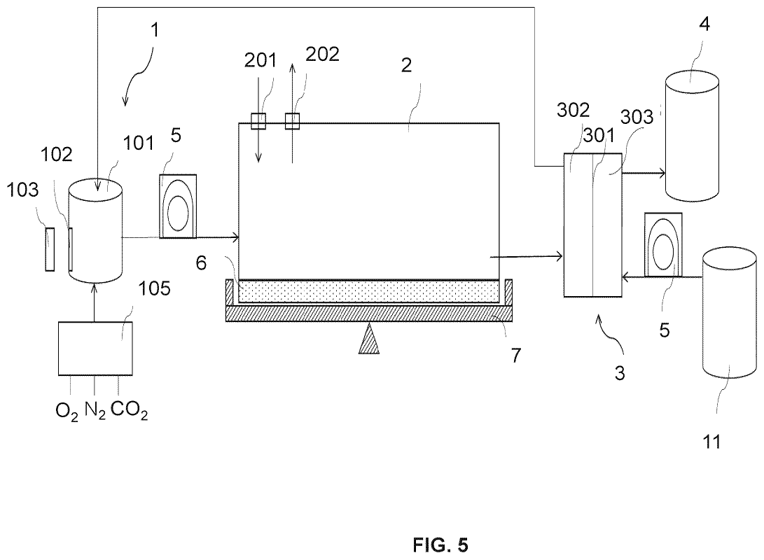

In some embodiments, referring to FIGS. 5 and 6, the fluid replenishment unit 11 may be connected with the second exchange chamber 303, and the fluid replenishment unit 11 may deliver one or more components required for the culture to the second exchange chamber 303. In some embodiments, the fluid replenishment unit 11 may deliver a nutrient solution having a nutrient component to the second exchange chamber 303. In some embodiments, the components of the nutrient solution delivered by the fluid replenishment unit 11 to the second exchange chamber 303 may be capable of permeating through the membrane assembly 301 into the first exchange chamber 302. In some embodiments, the power unit 5 may be provided between the fluid replenishment unit 11 and the second exchange chamber 303, and the power unit 5 may control a rate at which the fluid replenishment unit 11 delivers the nutrient solution into the second exchange chamber 303. Since the concentration of the nutrient component contained in the nutrient solution delivered to the second exchange chamber 303 is greater than the concentration of the nutrient component of the culture fluid in the first exchange chamber 302, a concentration difference between the two may drive the nutrient component in the nutrient solution to penetrate through the membrane assembly 301 into the first exchange chamber 302. In some embodiments, the culture fluid in the first exchange chamber 302 may be delivered to the culture chamber 2 for consumption by growth of the culture. In some embodiments, the culture fluid in the first exchange chamber 302 may also be delivered into the mixing chamber 101, and the culture fluid may be further mixed with the nutrient solution introduced from the fluid replenishment chamber 104 and the gas introduced from the gas mixing control unit 105 to form a culture fluid that is more suitable for the growth of the culture.

In some embodiments, due to the different consumption rate of individual components in the culture fluid during the cultivation process, one or more components may be added directionally by the fluid replenishment unit 11 to ensure balance of the individual components and reduce the overall fluid change step, the cost, and the chances of contamination of the culture at the same time. The fluid replenishment unit 11 may replenish the liquid to the second exchange chamber 303, and then the nutrient solution may be delivered to the culture chamber 2 from the first exchange chamber 302, therefore, the concentration and the components of the nutrient component of the nutrient solution in the fluid replenishment unit 11 are not formulated to be the same as the concentration and components of the culture fluid required for the culture, and the volume of the fluid replenishment unit 11 may be smaller. At the same time, a perfusion rate of the mixing chamber 101 may be higher without worrying about consuming the culture fluid too quickly due to the increased perfusion rate, improving the culture efficiency and increasing the variety of cultivable cultures.

In some embodiments, the fluid replenishment unit 11 may also be connected with the first exchange chamber 302, and the fluid replenishment unit 11 may deliver one or more components required for the culture to the first exchange chamber 302. In some embodiments, when the metabolite concentration of the culture fluid in the first exchange chamber 302 is higher than the preset threshold, the fluid replenishment unit 11 may deliver a nutrient solution having a nutrient composition to the first exchange chamber 302. In this way, the metabolite concentration of the culture fluid in the first exchange chamber 302 may be reduced below the preset threshold so that the culture fluid in the first exchange chamber 302 may be delivered to the culture chamber 2 or the mixing chamber 101 for reuse.

In some embodiments, the culture fluid circulation module 140 may further include a second exchange unit disposed in the culture chamber 2 for exchanging components of the culture fluid in the culture chamber 2 and circulating at least a portion of the components of the culture fluid.

In some embodiments, a count of the culture chamber 2 may include a plurality. In some embodiments, the plurality of culture chambers may form the second exchange unit. In some embodiments, the second exchange unit may perform a component exchange of the culture fluid in the chamber holding the culture, and reuse a portion of the culture fluid after the component exchange. In some embodiments, a portion of the culture fluid after the component exchange may be refluxed into the mixing chamber 101. In some embodiments, a portion of the culture fluid after the component exchange may also be refluxed into the culture chamber for holding the culture.

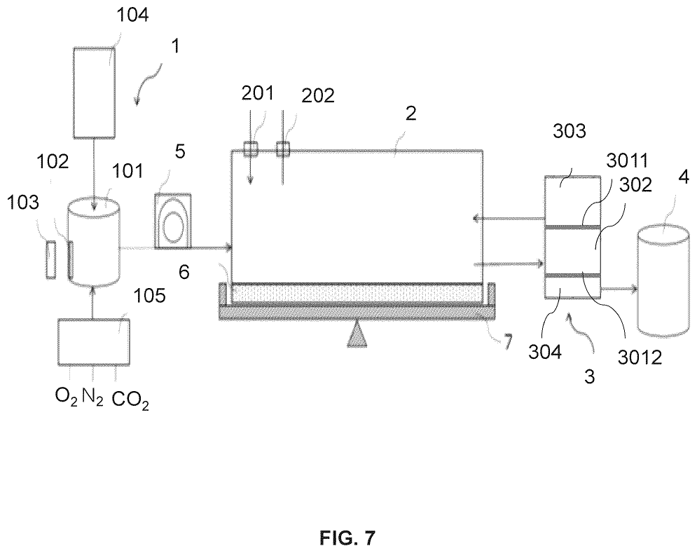

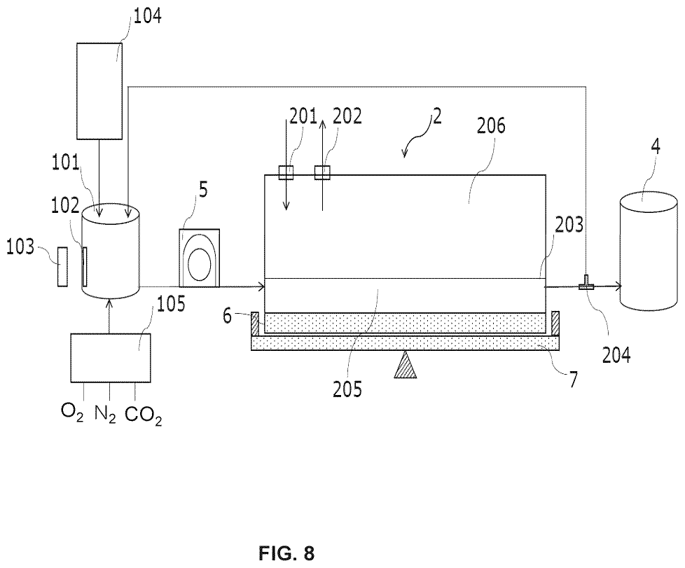

In some embodiments, referring to FIGS. 8-9, the second exchange unit may include a first culture chamber 205 and a second culture chamber 206 disposed in the culture chamber 2, and a first membrane assembly 203 disposed between the first culture chamber 205 and the second culture chamber 206, and at least a portion of the components of the culture fluid may be capable of penetrating through the first membrane assembly 203 by permeation. In some embodiments, the first culture chamber 205 and the second culture chamber 206 may be disposed up and down in the culture chamber 2. In some embodiments, the first culture chamber 205 and the second culture chamber 206 may be disposed left and right in the culture chamber 2. In some embodiments, the first culture chamber 205 and the second culture chamber 206 may be disposed front and rear in the culture chamber 2. In some embodiments, the first culture chamber 205 and the second culture chamber 206 may be disposed inside and outside in the culture chamber 2.

In some embodiments, the second culture chamber 206 may be used to hold the culture and cultivate the culture. In some embodiments, the first culture chamber 205 may be connected with the mixing chamber 101, and the mixing chamber 101 may be capable of delivering a culture fluid to the first culture chamber 205. After the culture fluid in the mixing chamber 101 is delivered into the first culture chamber 205, the nutrient composition in the culture fluid may permeate through the first membrane assembly 203 into the second culture chamber 206 for absorption and utilization by the culture. The metabolic waste produced by the growth of the culture may permeate through the first membrane assembly 203 into the first culture chamber 205. Since the first membrane assembly 203 is provided between the first culture chamber 205 and the second culture chamber 206, the culture fluid may be delivered to the first culture chamber 205 to prevent the culture fluid from directly washing the culture. In some embodiments, the pore size of the permeable pore of the first membrane assembly 203 may be smaller than an outer diameter of a single cell of the culture, causing that the first membrane assembly 203 is capable of retaining the culture and single dispersed cells in the culture are not washed away during the cultivation process, so as to make the system applicable to the cultivation of more species or modalities of cultures. In some embodiments, the pore size of the permeable pore of the first membrane assembly 203 may be in a range of 0.0001 microns to 10 microns. In some embodiments, the pore size of the permeable pore of the first membrane assembly 203 may be in a range of 0.0001 microns to 100 microns. In some embodiments, the pore size of the permeable pore of the first membrane assembly 203 may be in a range of 0.0001 microns to 50 microns. In some embodiments, the pore size of the permeable pore of the first membrane assembly 203 may be in a range of 0.0001 microns to 30 microns. In some embodiments, the pore size of the permeable pore of the first membrane assembly 203 may be in a range of 0.0001 microns to 20 microns.

In some embodiments, the first culture chamber 205 may include a fluid inlet and a fluid outlet. The fluid inlet may be used to receive the culture fluid from the other chambers, and the fluid outlet may be used to deliver the culture fluid to the other chambers. In some embodiments, the fluid inlet may be connected with the mixing chamber 101 in the culture fluid provision module 120 such that the first culture chamber 205 is capable of receiving the culture fluid from the culture fluid provision module 120. In some embodiments, the fluid outlet may be selectively connected with the mixing chamber 101 in the culture fluid provision module 120 such that the culture fluid in the first culture chamber 205 is capable of controllably flowing into the culture fluid provision module 120.

In some embodiments, the selectable connection may be a selection of connection or non-connection based on a preset condition. In some embodiments, the preset condition may be whether the metabolite concentration of the culture fluid is above the preset threshold (e.g., 5 mg/mL). In some embodiments, when the metabolite concentration of the culture fluid is above the preset threshold, the fluid outlet may be not connected with the mixing chamber 101 in the culture fluid provision module 120; when the metabolite concentration of the culture fluid is not higher than the preset threshold, the fluid outlet may be connected with the mixing chamber 101 in the culture fluid provision module 120. In some embodiments, the preset threshold of the metabolite concentration in the culture fluid may be in a range of 0.1 mg/mL to 10 mg/mL.

In some embodiments, the selectable connection may be achieved by a valve. In some embodiments, a metabolite concentration detection unit may be provided in the first culture chamber 205 for detecting a metabolite concentration of the culture fluid in the first culture chamber 205. In some embodiments, the control module 150 may control the second exchange unit to open or close the fluid outlet (e.g., a valve) based on a detection result of the metabolite concentration detection unit.

In some embodiments, the power unit 5 may be provided between the fluid inlet of the first culture chamber 205 and the mixing chamber 101. In some embodiments, the power unit 5 may control the flow rate of the culture fluid through the fluid inlet. In some embodiments, the power unit 5 may deliver the culture fluid in the mixing chamber 101 into the first culture chamber 205. In some embodiments, the power unit 5 may be provided between the fluid outlet and the mixing chamber 101. In some embodiments, the power unit 5 may control the flow rate of the culture fluid through the fluid outlet. In some embodiments, the power unit 5 may deliver the culture fluid in the first culture chamber 205 into the mixing chamber 101.

In some embodiments, the culture cultivated in the second culture chamber 206 may be an immune cell, and the immune cell may be cultivated in the second culture chamber 206 by the suspension culture. In some embodiments, the immune cell may include, but is not limited to, a T cell, an NK cell, etc. In some embodiments, referring to FIG. 8, the second culture chamber 206 may not include a fluid inlet and a fluid outlet, and the culture fluid in the second culture chamber 206 may be replenished and exchanged through permeation of the first membrane assembly 203, which has a low flow rate, so as to prevent the culture in the second culture chamber 206 from being washed away and simulate a micro-environment of the immune cell in the patient's body, improving the accuracy of the growth of the culture.

In some embodiments, the second culture chamber 206 and the first culture chamber 205 may be used to simultaneously cultivate the culture. In some embodiments, the cultures cultivated in the second culture chamber 206 and the first culture chamber 205 may be different. In some embodiments, the culture cultivated in the second culture chamber 206 may be an immune cell, and the cultivation manner may be a suspension cultivation. In some embodiments, the culture cultivated in the first culture chamber 205 may be an organoid cell, and the cultivation manner may be a support culture (e.g., the culture is placed into a matrix gel for cultivation). In some embodiments, referring to FIG. 8, the second culture chamber 206 may not include a fluid inlet and a fluid outlet, and the flow rate of the culture fluid in the second culture chamber 206 may be small to simulate the micro-environment of the immune cell in the patient's body; the first culture chamber 205 may include a fluid inlet and a fluid outlet, and the flow rate of the culture fluid in the first culture chamber 205 may be large to simulate a blood flow environment of the organoid cell in the patient's body, improving the accuracy of the growth of the culture.

In some embodiments, the first culture chamber 205 may include two fluid outlets. In some embodiments, one of the two fluid outlets may be selectively connected with the mixing chamber 101 and the other fluid outlet may be selectively connected with the collection unit 4. The collection unit 4 may be used to collect the culture fluid discharged from the first culture chamber 205. In some embodiments, when the metabolite concentration of the culture fluid in the first culture chamber 205 is higher than the preset threshold, the control module 150 may control the second exchange unit to open the fluid outlet that is selectively connected with the collection unit 4 and close the fluid outlet that is selectively connected with the mixing chamber 101, so that the culture fluid flowing out of the first culture chamber 205 is discharged into the collection unit 4. In some embodiments, when the metabolite concentration of the culture fluid in the first culture chamber 205 is not higher than the preset threshold, the control module 150 may control the second exchange unit to close the fluid outlet that is selectively connected with the collection unit 4 and open the fluid outlet that is selectively connected with the mixing chamber 101, so that the culture fluid flowing out of the first culture chamber 205 is delivered into the mixing chamber 101. In some embodiments, the preset threshold of the metabolite concentration in the culture fluid may be in a range of 0.1 mg/mL to 10 mg/mL.

In some embodiments, the first culture chamber 205 may also include only a fluid outlet that is selectively connected with both the mixing chamber 101 and the collection unit 4. In some embodiments, the fluid outlet may be selectively connected with both the mixing chamber 101 and the collection unit 4 by providing a three-way valve. In some embodiments, three interfaces of the three-way valve 204 may be connected with the fluid outlet, the collection unit 4, and the mixing chamber 101 via pipelines, respectively. The first culture chamber 205 may be connected with the collection unit 4, or the first culture chamber 205 may be connected with the mixing chamber 101 by switching the three-way valve. In some embodiments, when the metabolite concentration of the culture fluid in the first culture chamber 205 is higher than the preset threshold, an interface of the three-way valve connected with the collection unit 4 may be opened and an interface connected with the mixing chamber 101 may be closed, and the culture fluid in the first culture chamber 205 may be capable of being delivered into the collection unit 4 via the three-way valve 204. In some embodiments, when the metabolite concentration of the culture fluid in the first culture chamber 205 is not higher than the preset threshold, the interface of the three-way valve connected with the collection unit 4 may be closed and the interface connected with the mixing chamber 101 may be opened, and the culture fluid in the first culture chamber 205 may be capable of being delivered into the mixing chamber 101 via the three-way valve 204. In some embodiments, the power unit 5 may be provided between the three-way valve 204 and the fluid outlet to control the flow rate of the culture fluid through the fluid outlet.

In some embodiments, the first culture chamber 205 may include a fluid inlet and the second culture chamber 206 may include a fluid outlet. In some embodiments, the fluid inlet may be connected with the mixing chamber 101 in the culture fluid provision module 120 so that the first culture chamber 205 may receive the culture fluid from the mixing chamber 101. In some embodiments, the fluid outlet may be selectively connected with the mixing chamber 101 in the culture fluid provision module 120. In some embodiments, the fluid outlet may also be selectively connected with the collection unit 4. In some embodiments, when the metabolite concentration of the culture fluid in the second culture chamber 206 is not above a preset threshold, the culture fluid in the second culture chamber 206 may flow into the mixing chamber 101. In some embodiments, when the metabolite concentration of the culture fluid in the second culture chamber 206 is above the preset threshold, the culture fluid in the second culture chamber 206 may flow into the collection unit 4.

In some embodiments, the first culture chamber 205 may include two fluid inlets and the second culture chamber 206 may include a fluid outlet. In some embodiments, one of the fluid inlets may be connected with the mixing chamber 101 in the culture fluid provision module 120, such that the first culture chamber 205 may receive the culture fluid from the mixing chamber 101. In some embodiments, the other fluid inlet may be selectively connected with the fluid outlet of the second culture chamber 206, such that the first culture chamber 205 may selectively receive the culture fluid from the second culture chamber 206. In some embodiments, the fluid outlet of the second culture chamber 206 may also be selectively connected with the collection unit 4, so that when the metabolite concentration of the culture fluid in the second culture chamber 206 is higher than the preset threshold, the culture fluid is delivered to the collection unit 4. In some embodiments, the first culture chamber 205 may further include a fluid outlet, so that when there is too much culture fluid in the first culture chamber 205, the culture fluid is discharged, causing that the introduced amount and discharged amount of the culture fluid in the first culture chamber 205 are balanced. In some embodiments, the fluid outlet of the first culture chamber 205 may be selectively connected with the collection unit 4, so that when there is too much culture fluid in the first culture chamber 205, the culture fluid may be delivered to the collection unit 4.

In some embodiments, the second culture chamber 206 may be connected with the mixing chamber 101, and the mixing chamber 101 may be capable of delivering the culture fluid to the second culture chamber 206. In some embodiments, the second culture chamber 206 may include a fluid inlet, and the mixing chamber 101 may deliver the culture fluid to the second culture chamber 206 through the fluid inlet of the second culture chamber 206. In some embodiments, the second culture chamber 206 may include a fluid outlet. In some embodiments, the fluid outlet may be selectively connected with both the collection unit 4 and the mixing chamber 101. When the metabolite concentration of the culture fluid in the second culture chamber 206 is higher than the preset threshold, the culture fluid may be delivered to the collection unit 4; when the metabolite concentration of the culture fluid in the second culture chamber 206 is not higher than the preset threshold, the culture fluid may be delivered to the mixing chamber 101. In some embodiments, the first culture chamber 205 may include a fluid inlet and a fluid outlet. In some embodiments, the fluid inlet of the first culture chamber 205 may be connected with the mixing chamber 101, and the fluid outlet of the first culture chamber 205 may be connected with the collection unit 4. In some embodiments, the delivery of the culture fluid to the first culture chamber 205 through the mixing chamber 101 may cause the concentration of the nutrient composition of the culture fluid in the first culture chamber 205 to be higher than the concentration of the nutrient composition of the culture fluid in the second culture chamber 206, and the metabolite concentration of the culture fluid in the first culture chamber 205 to be lower than the metabolite concentration of the culture fluid in the second culture chamber 206, so that the nutrient composition of the culture fluid in the first culture chamber 205 is more easily permeable to the culture fluid in the second culture chamber 206, and the metabolite of the culture fluid in the second culture chamber 206 is more easily permeable to the culture fluid in the first culture chamber 205.

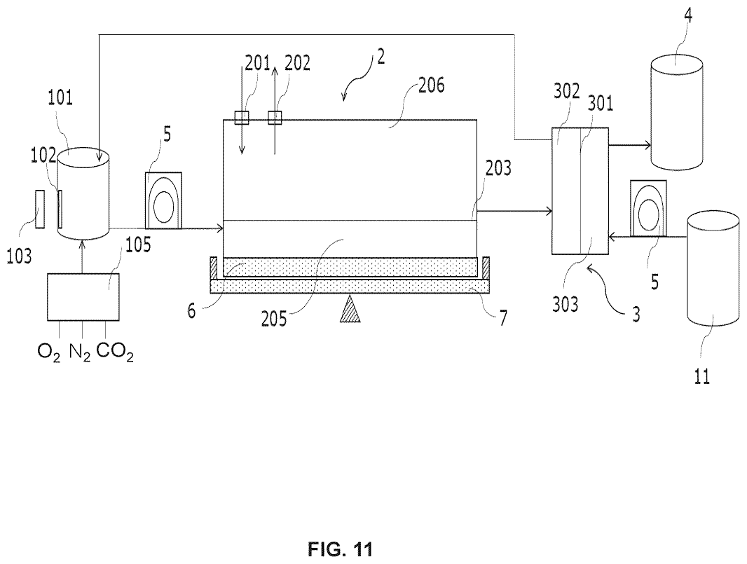

In some embodiments, referring to FIG. 12, the fluid inlet of the first culture chamber 205 may not be connected with the mixing chamber 101, but may be connected with the fluid replenishment unit 11. In some embodiments, the fluid replenishment unit may be capable of delivering the nutrient solution containing the nutrient composition into the first culture chamber 205. Since the concentration and composition of the nutrient solution in the fluid replenishment unit 11 are not formulated to be the same as the concentration and composition of the culture fluid required for the culture, a volume of the fluid replenishment unit 11 may be smaller as compared to the mixing chamber 101, making the in vitro life culture system 100 less cost.

In some embodiments, the fluid replenishment unit 11 may be connected with the fluid inlet of the first culture chamber 205, the fluid replenishment unit 11 contains a nutrient solution, and the fluid replenishment unit 11 may deliver the nutrient solution to the first culture chamber 205. In some embodiments, the power unit 5 may be provided between the fluid replenishment unit 11 and the first culture chamber 205, and the power unit 5 may control a rate at which the fluid replenishment unit 11 delivers the nutrient solution into the first culture chamber 205. In some embodiments, the nutrient solution in the first culture chamber 205 may be capable of permeating through the first membrane assembly 203 into the second culture chamber 206. Since the concentration of the nutrient composition in the nutrient solution is greater than the concentration of the nutrient composition of the culture fluid in the second culture chamber 206, a concentration difference between the two may drive the composition in the nutrient solution to penetrate through the first membrane assembly 203 into the second culture chamber 206 to replenish the nutrient composition required for the growth of the culture in the second culture chamber 206.

In some embodiments, the functions and structures of the first culture chamber 205 and the second culture chamber 206 may be interchangeable. In some embodiments, connection relationships between the first culture chamber 205/the second culture chamber 206 and other components of the in vitro life culture system 100 may also be interchangeable. In some embodiments, referring to FIG. 12, the first culture chamber 205 may be used to place and cultivate a culture, and the component exchange of a culture fluid may be performed between the second culture chamber 206 and the first culture chamber 205. In some embodiments, the second culture chamber 206 may include a fluid inlet and a fluid outlet. In some embodiments, the fluid replenishment unit 11 may be connected with the fluid inlet of the second culture chamber 206 for delivering the nutrient solution into the second culture chamber 206, and the collection unit 4 may be connected with the fluid outlet of the second culture chamber 206 for collecting the culture fluid discharged from the second culture chamber 206.

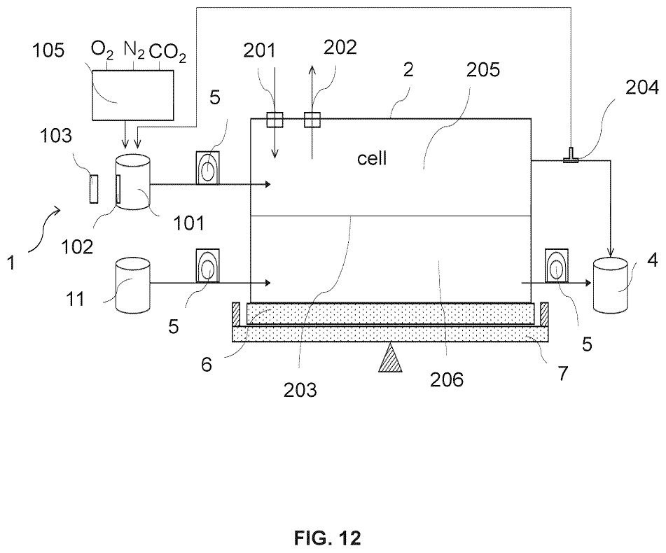

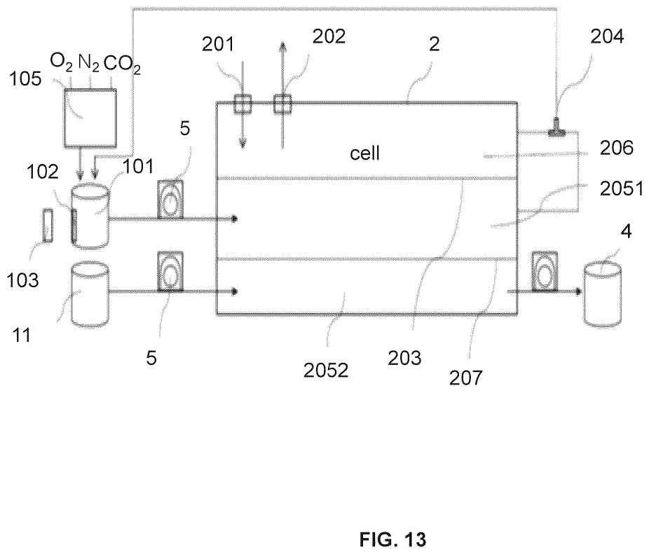

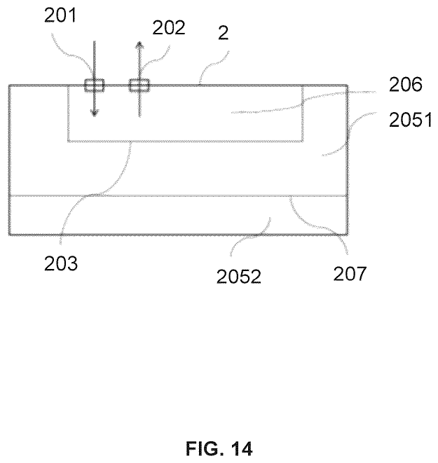

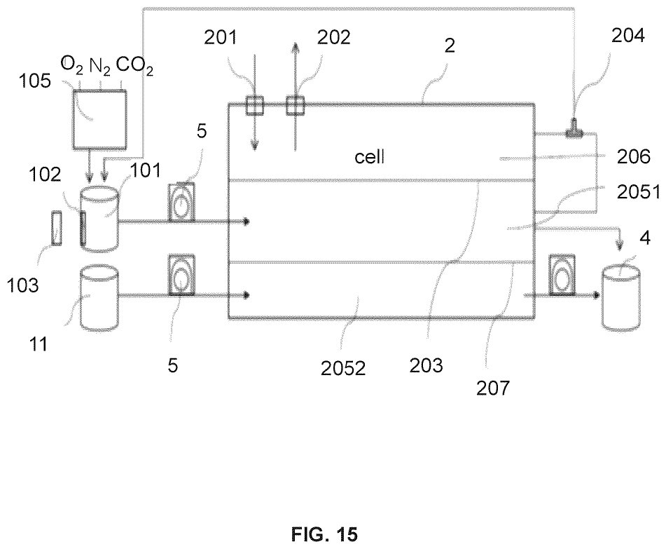

In some embodiments, referring to FIGS. 13-16, a third membrane assembly 207 may be provided in the first culture chamber 205, the third membrane assembly 207 may divide the first culture chamber 205 into a first sub-culture chamber 2051 and a second sub-culture chamber 2052, and the component exchange may be performed between the first sub-culture chamber 2051 and the second sub-culture chamber 2052 via the third membrane assembly 207.

In some embodiments, the third membrane assembly 207 may also be used to retain or permeate the component in the culture fluid. In some embodiments, the third membrane assembly 207 may be also capable of retaining the culture, so that single dispersed cells of the culture are not washed away, making the system applicable to cultivating more types or modalities of cultures.

In some embodiments, the second culture chamber 206 may be provided adjacent to the first sub-culture chamber 2051 and the component exchange may be performed between the second culture chamber 206 and the first sub-culture chamber 2051 through the first membrane assembly 203. In some embodiments, the second culture chamber 206 may be provided adjacent to the second sub-culture chamber 2052 and the component exchange may be performed between the second culture chamber 206 and the second sub-culture chamber 2052 through the first membrane assembly 203.

In some embodiments, the second culture chamber 206 may be used to place and cultivate a culture. In some embodiments, the first sub-culture chamber 2051 may be connected with the mixing chamber 101 for receiving the culture fluid from the mixing chamber 101. In some embodiments, the first sub-culture chamber 2051 may include a first fluid inlet for receiving the culture fluid flowing into the first sub-culture chamber 2051. In some embodiments, the mixing chamber 101 may be connected with the first fluid inlet. Due to the consumption of the culture fluid by the culture in the second culture chamber 206 during the cultivation process, the concentration of the nutrient composition required for the culture of the culture fluid in the second culture chamber 206 is lower than that in the first sub-culture chamber 2051 and the metabolite concentration of the culture fluid in the second culture chamber 206 is higher than that in the first sub-culture chamber 2051; the nutrient composition of the culture fluid in the first sub-culture chamber 2051 may permeate through the first membrane assembly 203 into the second culture chamber 206 for absorption and utilization by the culture, and the metabolic waste in the second culture chamber 206 may permeate through the first membrane assembly 203 into the first sub-culture chamber 2051. In some embodiments, the first sub-culture chamber 2051 may further include a first fluid outlet for flowing out the culture fluid from the first sub-culture chamber 2051. In some embodiments, the first fluid outlet may be selectively connected with the second culture chamber 206, when the metabolite concentration of the culture fluid in the first sub-culture chamber 2051 is not higher than the preset threshold, the first fluid outlet may be connected with the second culture chamber 206, and when the metabolite concentration of the culture fluid in the first sub-culture chamber 2051 is higher than the preset threshold, the first fluid outlet may not be connected with the second culture chamber 206. In some embodiments, the first fluid outlet may also be selectively connected with the mixing chamber 101, when the metabolite concentration of the culture fluid in the first sub-culture chamber 2051 is not higher than the preset threshold, the first fluid outlet may be connected with the mixing chamber 101, and when the metabolite concentration of the culture fluid in the first sub-culture chamber 2051 is higher than the preset threshold, the first fluid outlet may not be connected with the mixing chamber 101. In some embodiments, the first fluid outlet may also be selectively connected with the collection unit 4, when the metabolite concentration of the culture fluid in the first sub-culture chamber 2051 is not higher than the preset threshold, the first fluid outlet may not be connected with the collection unit 4, and when the metabolite concentration of the culture fluid in the first sub-culture chamber 2051 is higher than the preset threshold, the first fluid outlet may be connected with the collection unit 4. Since the nutrient composition of the culture fluid in the first sub-culture chamber 2051 permeates into the second culture chamber 206 and the culture fluid in the first sub-culture chamber 2051 is mixed with the metabolite from the second culture chamber 206, the concentration of the nutrient composition of the culture fluid in the first sub-culture chamber 2051 is lower than that in the second sub-culture chamber 2052 and the metabolite concentration of the culture fluid in the first sub-culture chamber 2051 is higher than that in the second sub-culture chamber 2052; the nutrient composition of the culture fluid in the second sub-culture chamber 2052 may permeate through the third membrane assembly 207 into the first sub-culture chamber 2051, and the metabolic waste in the first sub-culture chamber 2051 may permeate through the third membrane assembly 207 into the second sub-culture chamber 2052. In some embodiments, the second sub-culture chamber 2052 may include a second fluid outlet for flowing out the culture fluid from the second sub-culture chamber 2052. In some embodiments, the second fluid outlet may be connected with the collection unit 4.

In some embodiments, the second sub-culture chamber 2052 may include a second fluid inlet for flowing the culture fluid into the second sub-culture chamber 2052. In some embodiments, the second fluid inlet may be connected with the mixing chamber 101. In some embodiments, the second fluid inlet may also not be connected with the mixing chamber 101, but be connected with the fluid replenishment unit 11. The fluid replenishment unit 11 may deliver a nutrient solution containing one or more components (e.g., nutrient composition) required for the culture to the second sub-culture chamber 2052 via the second fluid inlet.

In some embodiments, the fluid replenishment unit 11 may be connected with the first sub-culture chamber 2051 for delivering one or more components required for the culture to the first sub-culture chamber 2051 to replenish the one or more nutrient components in the culture fluid in the first sub-culture chamber 2051, ensuring the integrity of the one or more nutrient components in the culture fluid.

In some embodiments, the fluid replenishment unit 11 may also be connected with the second sub-culture chamber 2052 for delivering the one or more components required by the culture to the second sub-culture chamber 2052. In some embodiments, the concentration of the nutrient solution in the fluid replenishment unit 11 may be greater than the concentration of the culture fluid in the second sub-culture chamber 2052, and a concentration difference between the two may drive components of the nutrient solution to penetrate through the third membrane assembly 207 into the first sub-culture chamber 2051 to replenish the nutrient components required for growth of the culture in the culture fluid of the first sub-culture chamber 2051. By delivering the nutrient solution to the second sub-culture chamber 2052, the metabolite concentration of the culture fluid in the second sub-culture chamber 2052 may be reduced to promote the penetration of the metabolite in the first sub-culture chamber 2051 into the second sub-culture chamber 2052, improving the efficiency of the component exchange.

In some embodiments, the fluid replenishment unit 11 may be connected with both the first sub-culture chamber 2051 and the second sub-culture chamber 2052 for simultaneously delivering the one or more components required for the culture to both the first sub-culture chamber 2051 and the second sub-culture chamber 2052.

In some embodiments, the power unit 5 may be provided between the second fluid inlet and the mixing chamber 101 or the fluid replenishment unit 11. In some embodiments, the power unit 5 may control the flow rate of the culture fluid or nutrient solution in a fluid channel. In some embodiments, the power unit 5 may also open or close the fluid channel. In some embodiments, when the metabolite concentration of the culture fluid in the second sub-culture chamber 2052 is higher than the preset threshold, the power unit 5 may open the fluid channel to deliver a certain flow volume (e.g., 100 mL, etc.) of the culture fluid or nutrient solution from the mixing chamber 101 or the fluid replenishment unit 11 into the second sub-culture chamber 2052 for diluting the metabolite concentration of the culture fluid in the second sub-culture chamber 2052. In some embodiments, the power unit 5 may close the fluid channel when the metabolite concentration of the culture fluid in the second sub-culture chamber 2052 is not higher than the preset threshold.

In some embodiments, the power unit 5 may also be provided between the second fluid outlet and the collection unit 4 for controlling the flow rate of the culture fluid at the second outlet. In some embodiments, the power unit 5 may also be provided between the first fluid inlet and the mixing chamber 101. In some embodiments, a first power unit may be provided between the culture fluid provision module 120 and the first fluid inlet, and a second power unit may be provided between the second fluid outlet and the collection unit. The first power unit and the second power unit may be configured to cause the flow rates of the culture fluid in the first sub-culture chamber 2051 and the second sub-culture chamber 2052 different.

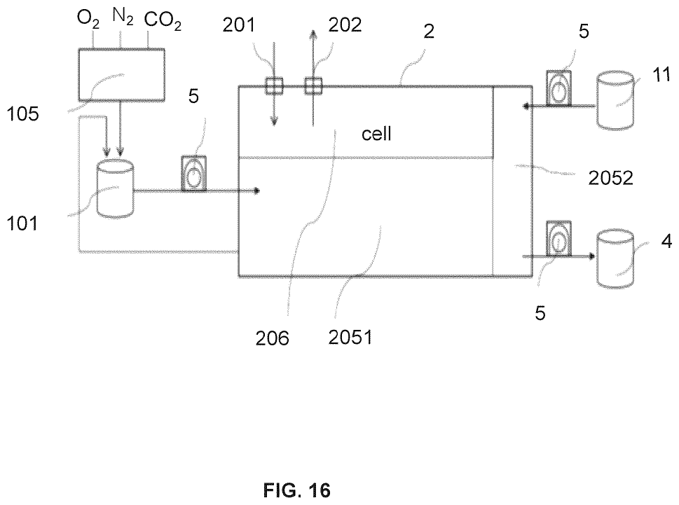

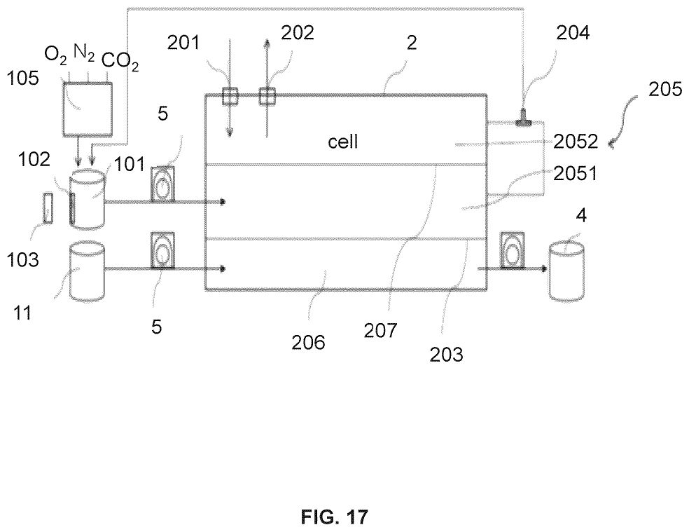

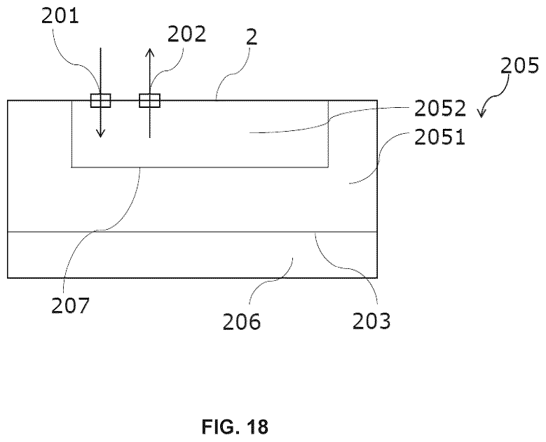

In some embodiments, positions, functions, and structures of the second culture chamber 206 and the second sub-culture chamber 2052 may be interchangeable. In some embodiments, referring to FIGS. 17-20, the second sub-culture chamber 2052 may be used to place and cultivate a culture. In some embodiments, due to the consumption of the culture fluid by the culture in the second sub-culture chamber 2052 during the cultivation process, the concentration of the components required for the culture within the culture fluid in the second sub-culture chamber 2052 may be lower than the concentration of the components required for the culture within the culture fluid in the first sub-culture chamber 2051, and the concentration of metabolic waste produced by the culture in the second sub-culture chamber 2052 may be higher than the concentration of metabolic waste produced by the culture in the first sub-culture chamber 2051. The useful components of the culture fluid in the first sub-culture chamber 2051 may permeate through the third membrane assembly 207 into the second sub-culture chamber 2052 for absorption and utilization by the culture, and metabolic wastes in the second sub-culture chamber 2052 may permeate through the third membrane assembly 207 into the first sub-culture chamber 2051. In some embodiments, since the nutrient composition of the culture fluid in the first sub-culture chamber 2051 penetrates into the second sub-culture chamber 2052 and the culture fluid in the first sub-culture chamber 2051 is mixed with metabolites from the second sub-culture chamber 2052, the concentration of the nutrient composition required for the culture within the culture fluid in the first sub-culture chamber 2051 may be lower than the concentration of the nutrient composition required for the culture within the culture fluid in the second sub-culture chamber 206 and the metabolite concentration of the culture fluid in the first sub-culture chamber 2051 may be higher than the metabolite concentration of the culture fluid in the second sub-culture chamber 206; the nutrient composition of the culture fluid in the second culture chamber 206 may permeate through the first membrane assembly 203 into the first sub-culture chamber 2051, and the metabolic wastes in the first sub-culture chamber 2051 may permeate through the first membrane assembly 203 into the second culture chamber 206. In some embodiments, the fluid replenishment unit 11 may be connected with the second culture chamber 206. In some embodiments, the fluid replenishment unit 11 may be connected with the first sub-culture chamber 2051.

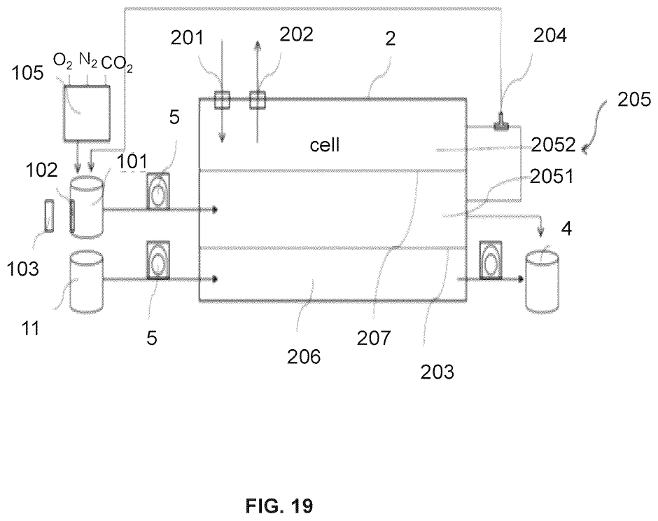

In some embodiments, referring to FIG. 20, both the first sub-culture chamber 2051 and the second sub-culture chamber 2052 may be connected with the second culture chamber 206 via the first membrane assembly 203. The fluid replenishment unit 11 may be connected with the second culture chamber 206, and the fluid replenishment unit 11 may be configured to store a nutrient solution for providing the nutrient solution to the second culture chamber 206. In some embodiments, the power unit 5 may be provided between the second culture chamber 206 and the fluid replenishment unit 11 for delivering the nutrient solution. The nutrient solution in the second culture chamber 206 may be capable of permeating through the first membrane assembly 203 into the first culture chamber 205 (specifically, permeating into the first sub-culture chamber 2051 and the second sub-culture chamber 2052). Since the concentration of the nutrient composition in the nutrient solution replenished by the fluid replenishment unit 11 is greater than the concentration of the nutrient composition of the culture fluid in the first sub-culture chamber 2051 and the second sub-culture chamber 2052, a concentration difference between the the concentration of the nutrient composition in the nutrient solution replenished by the fluid replenishment unit 11 and the concentration of the nutrient composition of the culture fluid in the first sub-culture chamber 2051 or the second sub-culture chamber 2052 may drive the nutrient composition in the nutrient solution penetrate through the first membrane assembly 203 into the first sub-culture chamber 2051 and the second sub-culture chamber 2052 to replenish the nutrient composition required for the growth of the cultures in the culture fluid of the first sub-culture chamber 2051 and the second sub-culture chamber 2052. In some embodiments, the second culture chamber 206 may be connected with the collection unit 4. Due to the concentration difference between the metabolite concentration of the culture fluid in the first culture chamber 205 and the metabolite concentration of the culture fluid in the second culture chamber 206, the metabolism waste produced by the culture contained in the culture fluid of the first culture chamber 205 (including the first sub-culture chamber 2051 and the second sub-culture chamber 2052) may permeate through the first membrane assembly 203 into the second culture chamber 206, the second culture chamber 206 may be connected with the collection unit 4, and the metabolism waste produced by the culture in the second culture chamber 206 may be discharged into the collection unit 4. In some embodiments, the nutrient solution in the second culture chamber 206 may be discharged when the metabolite concentration of culture fluid in the second culture chamber 206 is too high (referring to the metabolite concentration of culture fluid in the second culture chamber 206 larger than a metabolite concentration threshold) or the concentration of nutrient components required for the culture in the nutrient solution is too low (referring to the concentration of nutrient components required for the culture in the nutrient solution less than a concentration threshold of nutrient components), and then the nutrient solution may be replenished using the fluid replenishment unit 11. The first sub-culture chamber 2051 or the second sub-culture chamber 2052 may be capable of delivering the culture fluid to the mixing unit 1. In some embodiments, the culture fluid of the first sub-culture chamber 2051 may be capable of being delivered into the mixing chamber 101, which may enable the culture fluid of the first sub-culture chamber 2051 to be delivered into the mixing chamber 101 for mixing with gas to supplement the oxygen, and mixing with carbon dioxide or alkali solution to adjust the pH, so that the amount of dissolved oxygen and the pH of the culture fluid flowing from the mixing chamber 101 into the first sub-culture chamber 2051 meets the growth requirements of the culture.

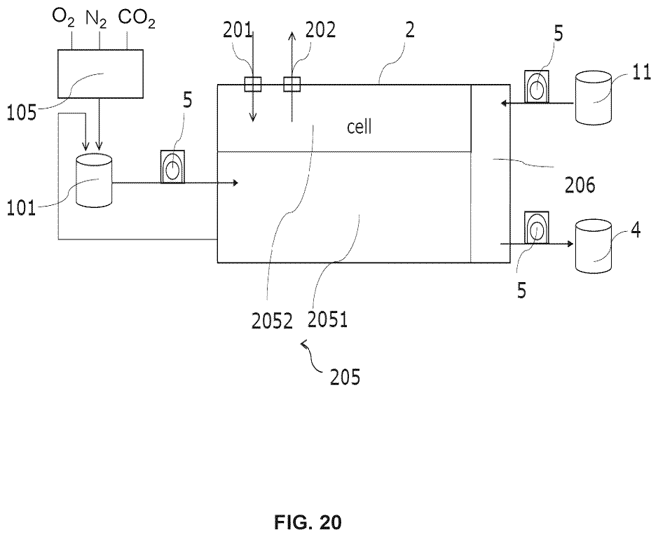

In some embodiments, referring to FIG. 21, the first sub-culture chamber 2051 may be used to place and cultivate a culture. The nutrient composition of the culture fluid in the first sub-culture chamber 2051 may permeate through the third membrane assembly 207 into the second sub-culture chamber 2052, and the metabolite in the culture fluid may permeate through the first membrane assembly 203 into the second culture chamber 206.

The third membrane assembly 207 may be used to retain or permeate components in the culture fluid, and the third membrane assembly 207 may be also capable of retaining the culture (specifically, cells of the culture), so that single dispersed cells of the culture are not washed away, making the system applicable to the cultivation of more cultures. Due to the consumption of the nutrient composition by the culture in the first sub-culture chamber 2051, the concentration of components (e.g., nutrients) required by the culture in the culture fluid of the first sub-culture chamber 2051 may be lower than that in the second sub-culture chamber 2052, and the metabolite concentration of the culture fluid of the first sub-culture chamber 2051 may be higher than that in the second sub-culture chamber 2052; the nutrient composition of the culture fluid in the second sub-culture chamber 2052 may permeate through the third membrane assembly 207 into the first sub-culture chamber 2051 for absorption and utilization by the culture, and the metabolite in the first sub-culture chamber 2051 may permeate through the third membrane assembly 207 into the second sub-culture chamber 2052.

The first sub-culture chamber 2051 may be connected with the second culture chamber 206 via the first membrane assembly 203. The fluid replenishment unit 11 may be connected with the second culture chamber 206, and the fluid replenishment unit 11 may be configured to store a nutrient solution for providing the nutrient solution to the second culture chamber 206. In some embodiments, the power unit 5 may be provided between the second culture chamber 206 and the fluid replenishment unit 11 for delivering the nutrient solution. The nutrient solution in the second culture chamber 206 may be capable of permeating through the first membrane assembly 203 into the first sub-culture chamber 2051. Since a concentration of the nutrient composition of the supplemented nutrient solution is greater than the concentration of the nutrient composition of the culture fluid in the first sub-culture chamber 2051, a concentration difference between the two may drive the nutrient composition in the nutrient solution to permeate through the first membrane assembly 203 into the first sub-culture chamber 2051 to replenish the nutrient composition required for the growth of the culture in the culture fluid of the first sub-culture chamber 2051.

In some embodiments, the second culture chamber 206 may be connected with the collection unit 4. Due to the concentration difference, the metabolite produced from the metabolism of the culture contained in the culture fluid of the first sub-culture chamber 2051 may permeate through the first membrane assembly 203 into the second culture chamber 206, and the second culture chamber 206 may be connected with the collection unit 4 for discharging the metabolite produced from the metabolism of the culture in the second culture chamber 206 into the collection unit 4. In some embodiments, when the metabolite concentration of the culture fluid in the second culture chamber 206 is too high or the concentration of the nutrient composition in the nutrient solution in the second culture chamber 206 is too low, the culture fluid in the second culture chamber 206 may be discharged, and then the second culture chamber 206 may be replenished with nutrient solution using the fluid replenishment unit 11.

In other embodiments, the first culture chamber 205 and the second culture chamber 206 may also both be connected with the collection unit 4, i.e., the first sub-culture chamber 2051 and/or the second sub-culture chamber 2052 may be connected with the collection unit 4, and the second culture chamber 206 may be connected with the collection unit 4. In some embodiments, both the first sub-culture chamber 2051 and the second culture chamber 206 may be connected with the collection unit 4. The metabolite produced from the metabolism of the culture contained in the culture fluid of the first sub-culture chamber 2051 may permeate through the first membrane assembly 203 into the second culture chamber 206. Metabolites produced by the cultures in both the first sub-culture chamber 2051 and the second culture chamber 206 may be discharged into the collection unit 4 and nutrient solution may be replenished into both the first sub-culture chamber 2051 and the second culture chamber 206.

The first sub-culture chamber 2051 and/or the second sub-culture chamber 2052 may be capable of delivering the culture fluid to the mixing unit 1. In some embodiments, the culture fluid in the first sub-culture chamber 2051 and the second culture chamber 206 may be capable of being delivered into the mixing chamber 101. The culture fluid in the first sub-culture chamber 2051 and the second sub-culture chamber 2052 may be capable of being delivered into the mixing chamber 101 for mixing with the gas to supplement the oxygen, and mixing with carbon dioxide or alkali solution to adjust the pH, so that the dissolved oxygen and the pH of the culture fluid flowing from the mixing chamber 101 into the second sub-culture chamber 2052 may meet the growth requirement of the culture.

In some embodiments, the culture fluid of the second culture chamber 206 may be capable of being delivered into the mixing chamber 101 to make the culture fluid flow into the mixing chamber 101 for reuse; and alternatively, as shown in FIG. 21, the culture fluid of the first sub-culture chamber 2051 may be delivered into the mixing chamber 101 to make the culture fluid flow into the mixing chamber 101 for reuse.

In some embodiments, the culture fluid in the first sub-culture chamber 2051 may be capable of being delivered to the second sub-culture chamber 2052. In some embodiments, the in vitro life culture system 100 may further include a three-way valve 204, and the three interfaces of the three-way valve 204 may be respectively connected with the first sub-culture chamber 2051, the second sub-culture chamber 2052, and the mixing chamber 101 via pipelines. The three-way valve 204 may be adjusted to deliver the culture fluid in the first sub-culture chamber 2051 to the second sub-culture chamber 2052, or deliver the culture fluid in the second sub-culture chamber 2052 to the mixing chamber 101, or deliver the culture fluid in the first sub-culture chamber 2051 to the mixing chamber 101.

As a consumption rate of each component in the culture fluid is different during the cultivation process, one or more components may be directionally added by the fluid replenishment unit 11 to ensure a balance of each component while reducing the overall fluid exchange step, costs and the chance of culture contamination. The fluid replenishment unit 11 may be configured to replenish the nutrient solution to the second culture chamber 206, and then the nutrient solution may be delivered from the second culture chamber 206 to the first culture chamber 205; therefore, the concentration and components of the nutrient solution in the fluid replenishment unit 11 are not formulated to be the same as the concentration and components of the culture fluid required for the culture, and the volume of the fluid replenishment unit 11 may be smaller. At the same time, the perfusion rate of the mixing chamber 101 may be higher without worrying about consuming the culture fluid too quickly due to the increased perfusion rate, improving the culture efficiency and increasing the variety of cultivable cultures.

In some embodiments, the concentration of each component in the culture fluid flowing out of the first culture chamber 205 (e.g., the first sub-culture chamber 2051, the second sub-culture chamber 2052) may be detected and components of the culture fluid may be targeted replenished by the fluid replenishment unit 11. A concentration detection may be performed by sampling detection or in-situ detection (e.g., an infrared spectroscopy, a fluorescence detection technique). In some embodiments, the in vitro life culture system 100 may also automatically add the nutrient composition to the fluid replenishment unit 11 based on a concentration detection result as described above.

In some embodiments, when the culture chamber 2 has a plurality of culture chambers, the sample addition opening 201 and the sample taking opening 202 may be provided on a chamber for placing the culture. In some embodiments, the culture may be placed in the first culture chamber 205, and the sample addition opening 201 and the sample taking opening 202 may be provided on the first culture chamber 205. In some embodiments, the culture may be placed in the second culture chamber 206, and the sample addition opening 201 and sample taking opening 202 may be provided on the second culture chamber 206. In some embodiments, the culture may be placed in the first sub-culture chamber 2051, and the sample addition opening 201 and the sample taking opening 202 may be provided on the first sub-culture chamber 2051. In some embodiments, the culture may be placed in the second sub-culture chamber 2052, and the sample addition opening 201 and the sample taking opening 202 may be provided on the second sub-culture chamber 2052.

In some embodiments, the culture fluid circulation module 140 may include a first exchange unit 3 located outside the culture chamber 2 and a second exchange unit located inside the culture chamber 2. In some embodiments, for more information about the specific structure of the first exchange unit 3, please refer to FIGS. 3-7 and its related descriptions, which is not repeated herein.

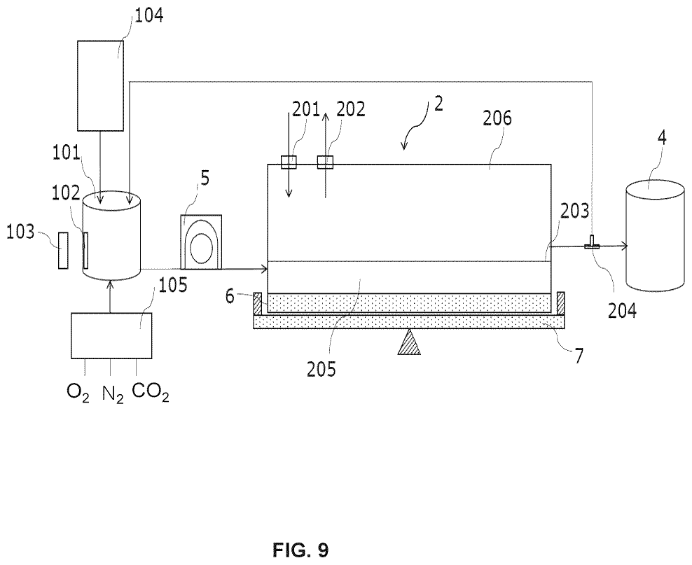

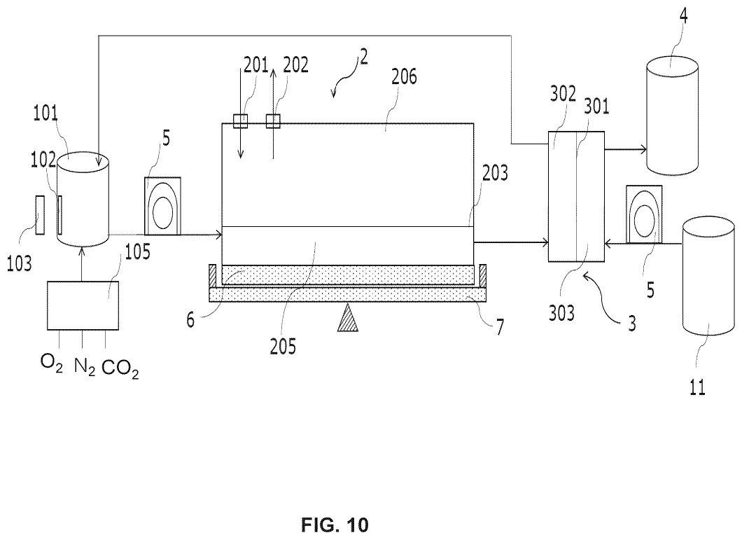

In some embodiments, referring to FIGS. 10-11, the first exchange unit 3 may include a first exchange chamber 302 and a second exchange chamber 303, and a membrane assembly 301 may be provided between the first exchange chamber 302 and the second exchange chamber 303. The culture chamber 2 may include a first culture chamber 205 and a second culture chamber 206, and the first membrane assembly 203 may be provided between the first culture chamber 205 and the second culture chamber 206, so that at least a portion of the components of the culture fluid may penetrate through the first membrane assembly 203 by permeation. The second exchange unit may correspond to the first culture chamber 205, and the second culture chamber 206 may be used for placing the culture.

In some embodiments, the first exchange chamber 302 may be connected with the first culture chamber 205 for receiving the culture fluid discharged from the first culture chamber 205. The first exchange chamber 302 may be connected with the second culture chamber 206 and/or the culture fluid provision module 120 for flowing the culture fluid into the second culture chamber 206 and/or the culture fluid provision module 120.

In some embodiments, the first exchange chamber 302 may be connected with the second culture chamber 206 for receiving the culture fluid discharged from the second culture chamber 206. The first exchange chamber 302 may be connected with the second culture chamber 206 and/or the culture fluid provision module 120 for flowing the culture fluid into the second culture chamber 206 and/or the culture fluid provision module 120.

In some embodiments, the first culture chamber 205 or the second culture chamber 206 may include a fluid outlet. In some embodiments, the fluid outlet of the first culture chamber 205 or the second culture chamber 206 and the mixing unit 1 may be connected with the first exchange chamber 302. In some embodiments, the first exchange chamber 302 may be provided with a first interface and a second interface, the fluid outlet of the first culture chamber 205 or the second culture chamber 206 may be connected with the first interface, and the mixing unit 1 may be connected with the second interface. The culture fluid in the first culture chamber 205 or the second culture chamber 206 may be capable of being delivered into the first exchange chamber 302 via the fluid outlet and the first interface. In some embodiments, in order to control the delivery rate and volume of the culture fluid between the first interface of the first exchange chamber 302 and the fluid outlet of the first culture chamber 205 or the second culture chamber 206, the power unit 5 may be provided between the first interface of the first exchange chamber 302 and the fluid outlet for delivering the culture fluid in the first culture chamber 205 into the first exchange chamber 302. The culture fluid in the first exchange chamber 302 may be capable of flowing into the mixing chamber 101 via the second interface, so that the nutrient composition of the culture fluid in the first exchange chamber 302 is circulated. In some embodiments, in order to control the delivery rate and volume of the culture fluid between the second interface of the first exchange chamber 302 and the mixing chamber 101, the power unit 5 may be provided between the second interface of the first exchange chamber 302 and the mixing chamber 101 for delivering the culture fluid in the first exchange chamber 302 to the mixing chamber 101 via the second interface.

In some embodiments, the collection unit 4 may be connected with the second exchange chamber 303, and the collection unit 4 may be used to collect the culture fluid discharged from the second exchange chamber 303. In some embodiments, the second exchange chamber 303 may be provided with a third interface and a fourth interface, the fourth interface being connected with the collection unit 4. The collection unit 4 may be connected with the second exchange chamber 303 for collecting the culture fluid from the second exchange chamber 303. In some embodiments, the power unit 5 may be provided between the collection unit 4 and the second exchange chamber 303 for delivering the culture fluid in the second exchange chamber 303 into the collection unit 4. In some embodiments, in order to control the delivery rate and the volume of the culture fluid between the second exchange chamber 303 and the collection unit 4, the power unit 5 may be provided between the second exchange chamber 303 and the collection unit 4 for delivering the culture fluid in the second exchange chamber 303 to the collection unit 4.

In some embodiments, the fluid replenishment unit 11 may be connected with the second exchange chamber 303. In some embodiments, the third interface may be connected with the fluid replenishment unit 11. The fluid replenishment unit 11 may be used to deliver a supplemental nutrient solution to the second exchange chamber 303, and at least a portion of components of the supplemental nutrient solution in the second exchange chamber 303 may be capable of permeating through the membrane assembly 301 into the first exchange chamber 302. The power unit 5 may be provided between the fluid replenishment unit 11 and the second exchange chamber 303. It may be determined whether to provide the power unit 5 between the fluid replenishment unit 11 and the second exchange chamber 303 for delivering the supplemental nutrient solution according to a relative setting position of the second exchange chamber 303 and the fluid replenishment unit 11 and the demand for the delivery rate and delivery volume of the supplemental nutrient solution. The concentration of the nutrient composition of the supplemental nutrient solution may be greater than the concentration of the nutrient composition of the culture fluid in the first exchange chamber 302, and the concentration difference between the concentration of the nutrient composition of the supplemental nutrient solution and the concentration of the nutrient composition of the culture fluid in the first exchange chamber 302 may drive the nutrient composition in the supplemental nutrient solution to permeate through the membrane assembly 301 into the first exchange chamber 302, then the supplemental nutrient solution may be delivered to the mixing chamber 101 for mixing with the gas to form a culture fluid.

As the consumption rate of each component in the culture fluid is different during the cultivation process, one or more components may be directionally added through the fluid replenishment unit 11 to ensure a balance of each component while reducing the overall fluid change step, the cost, and the possibility of the culture contamination. For example, only one or more components with a high consumption rate may be supplemented through the fluid replenishment unit 11. The fluid replenishment unit 11 may be configured to replenish the nutrient solution to the second exchange chamber 303, and then the nutrient solution may be delivered to the culture chamber 2 through the first exchange chamber 302; therefore, the concentration and composition of the nutrient solution in the fluid replenishment unit 11 are not formulated to be the same as the concentration and composition of the culture fluid required for the culture, and the volume of the fluid replenishment unit 11 may be smaller. At the same time, the perfusion rate of the mixing chamber 101 may be high without worrying about consuming the culture fluid too quickly due to the increased perfusion rate, improving the culture efficiency and increasing the variety of culturable cultures.

In order to control the temperature of the culture fluid in the culture chamber 2 to perform different operations on the culture at different temperatures during the cultivation process, in some embodiments, the culture module 110 may include a temperature control unit 6. In some embodiments, the temperature control unit may include a temperature controller. In some embodiments, the temperature control unit 6 may be used to control a temperature of the culture chamber 2 to a first temperature. The first temperature may correspond to a liquefaction temperature of a support structure of the culture. The liquefaction temperature is a temperature at which the support structure of the culture begins to change from a solid state to a liquid state. In some embodiments, the first temperature may be in a range of 0° C. to 40° C. In some embodiments, the first temperature may be in a range of 0° C. to 35° C. In some embodiments, the first temperature may be in a range of 0° C. to 30° C. In some embodiments, the first temperature may be in a range of 0° C. to 25° C. In some embodiments, the first temperature may be in a range of 0° C. to 20° C. In some embodiments, the first temperature may be in a range of 0° C. to 15° C. In some embodiments, the first temperature may be in a range of 0° C. to 10° C. In some embodiments, the first temperature may be in a range of 0° C. to 5° C. In some embodiments, the support structure of the culture may be used to form a network support of the culture, facilitating a 3D growth of the culture. In some embodiments, the support structure of the culture may include, but is not limited to, a matrix gel and a synthetic gel. In some embodiments, the matrix gel may be a basement membrane matrix extracted from an EHS mouse tumor enriched in extracellular matrix proteins. In some embodiments, the liquefaction temperature of the matrix gel may be in a range of −8° C. to 2° C. When the temperature is lowered from a high temperature to the range of −8° C. to 2° C., the matrix gel may be transformed from a solid state to a liquid state, and when the temperature is increased from a low temperature to the range of −8° C. to 2° C., the matrix gel may be solidified, and the matrix gel may provide a very important support for simulating the growth environment of cells in vitro based on its composition and the properties. In some embodiments, the synthetic gel may be a gel that is synthesized artificially, and the liquefaction temperature of the synthetic gel may be 25° C. at a room temperature, and when the temperature is lowered from a high temperature to 25° C., the synthetic gel may be transformed from a solid state to a liquid state, and when the temperature is increased from a low temperature to 25° C., the synthetic gel may be solidified. In some embodiments, the synthetic gel may include, but is not limited to, a block copolymer of poly(N-isopropylacrylamide) and poly(ethylene glycol) (PNIPAM-PEG), a block copolymer of polyethylene glycol (PEG) and poly(lactic-co-glycolic acid) (PLGA) (PEG-PLGA), a PLGA-PEG-PLGA triblock polymer, a PCLA-PEG-PCLA triblock copolymer (PCLA being a copolymer of ε-caprolactone and L-Lactide, a PCLA-PEG-PCLA grafted RGD peptide).

In some embodiments, the temperature control unit 6 may also be used to control the temperature of the culture chamber 2 to switch between the first temperature and a second temperature. The second temperature may correspond to a physiological temperature of the culture. The physiological temperature is a temperature suitable for the growth of organisms. In some embodiments, the second temperature may be in a range of 20° C. to 40° C. In some embodiments, the second temperature may be in a range of 25° C. to 40° C. In some embodiments, the second temperature may be in a range of 30° C. to 40° C. In some embodiments, the temperature control unit 6 may control the temperature of the culture chamber to be increased from the first temperature to the second temperature. In some embodiments, the temperature control unit 6 may control the temperature of the culture chamber to be reduced from the second temperature to the first temperature. In some embodiments, the first temperature may be lower than the second temperature.

In some embodiments, the culture fluid provision module 120 may also include a temperature control unit 6. In some embodiments, the mixing chamber 101 may be used to mix the nutrient solution and the gas to form a culture fluid, and the temperature control unit 6 may be used to control the temperature of the mixing chamber 101 to the second temperature, making the temperature of the culture fluid increase to the physiological temperature suitable for the growth of the culture to cultivate the culture.

In some embodiments, the temperature control unit 6 may be used to control the temperature of the mixing chamber 101 and the culture chamber 2. In some embodiments, the temperature control unit 6 may be used to heat the mixing chamber 101 and the culture chamber 2. The temperature control unit 6 may heat the mixing chamber 101 and the culture chamber 2 to raise the temperature of the mixing chamber 101 and the culture chamber 2 when the temperature of the mixing chamber 101 and the culture chamber 2 is too low, so as to raise the temperature of the culture fluid in the mixing chamber 101 and the temperature of the culture fluid in the culture chamber 2 to the physiological temperature suitable for growth of the culture to cultivate the culture. In some embodiments, the temperature control unit 6 may be used to cool the mixing chamber 101 and the culture chamber 2. The temperature control unit 6 may cool the mixing chamber 101 and the culture chamber 2 to lower the temperature of the mixing chamber 101 and the culture chamber 2 when the temperature of the mixing chamber 101 and the culture chamber 2 is too high, so as to lower the temperature of the culture fluid in the mixing chamber 101 and the temperature of the culture fluid in the culture chamber 2 to the physiological temperature suitable for growth of the culture to cultivate the culture. In some embodiments, the temperature control unit 6 may be used to control the temperature of the mixing chamber 101 and the culture chamber 2 to switch between the first temperature and the second temperature. In some embodiments, after the end of the cultivation, when the substrate gel is required to be removed and the culture is collected, the temperature control unit 6 may control the temperature of the culture fluid in the culture chamber 2 to the first temperature, and the culture may be taken through the sample taking opening 202 after the support structure of the culture is liquefied.

In some embodiments, the temperature control unit 6 may include a refrigeration assembly and a temperature control module, the refrigeration assembly may be electrically connected with the temperature control module. In some embodiments, the temperature control module may be used to control the refrigeration assembly to cool the culture chamber 2 to the first temperature. In some embodiments, the temperature of the culture chamber 2 may be restored to an ambient temperature when the refrigeration assembly stops working. In some embodiments, the ambient temperature may refer to a room temperature (e.g., 25° C.). In some embodiments, the ambient temperature may be the same as the physiological temperature of the culture in the set culture environment, and a range of values of the ambient temperature may be equal to the range of values of the physiological temperature of the culture. In some embodiments, the ambient temperature may be the same as the first temperature, and a cooling rate of the temperature of the culture chamber 2 may be accelerated by providing the refrigeration assembly, thereby improving the cooling efficiency. In some embodiments, a temperature control duration for controlling, by the temperature control module, the refrigeration assembly to cool the temperature of the culture chamber 2 to the first temperature may be manually set, e.g., setting the temperature control module to cool the temperature of the culture chamber 2 to the first temperature within 30 minutes. In some embodiments, the temperature control duration may be set according to the time change of day. In some embodiments, when the temperature is high during the period of the day, the temperature control duration may be set to be longer compared with the temperature being low during the period of the day, so that the temperature of the culture chamber 2 may be reduced to the first temperature more quickly.

In some embodiments, the temperature control unit 6 may further include a heating assembly, and the heating assembly may be electrically connected with the temperature control module. In some embodiments, the temperature control module may be used to control the heating assembly to heat the chamber. Specifically, the temperature control module may be used to control the heating assembly to heat the culture chamber 2 to the second temperature. In some embodiments, the temperature of the culture chamber 2 may be restored to the ambient temperature when the heating assembly stops working. In some embodiments, the ambient temperature may be the same as the second temperature, and a heating rate of the temperature of the culture chamber 2 may be accelerated by providing the heating component, thereby improving the efficiency of heating. In some embodiments, a working time length for controlling, by the temperature control module, the heating assembly to heat the temperature of the culture chamber 2 to the second temperature may be manually set, e.g., setting the temperature control module to heat the temperature of the culture chamber 2 to the second temperature within 30 minutes.

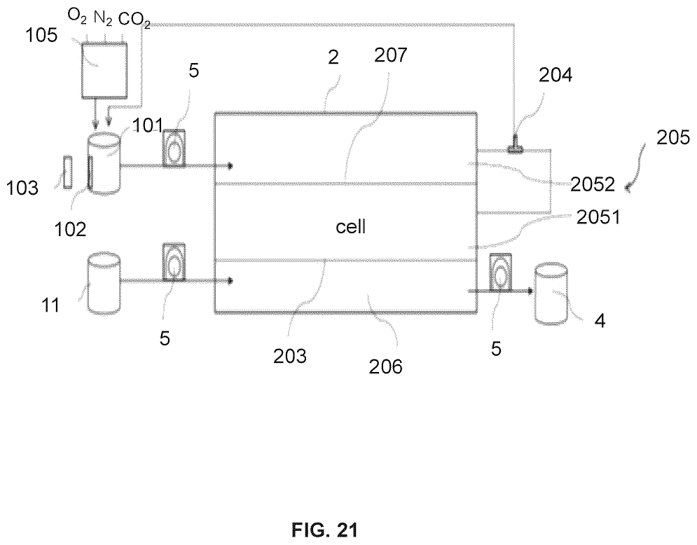

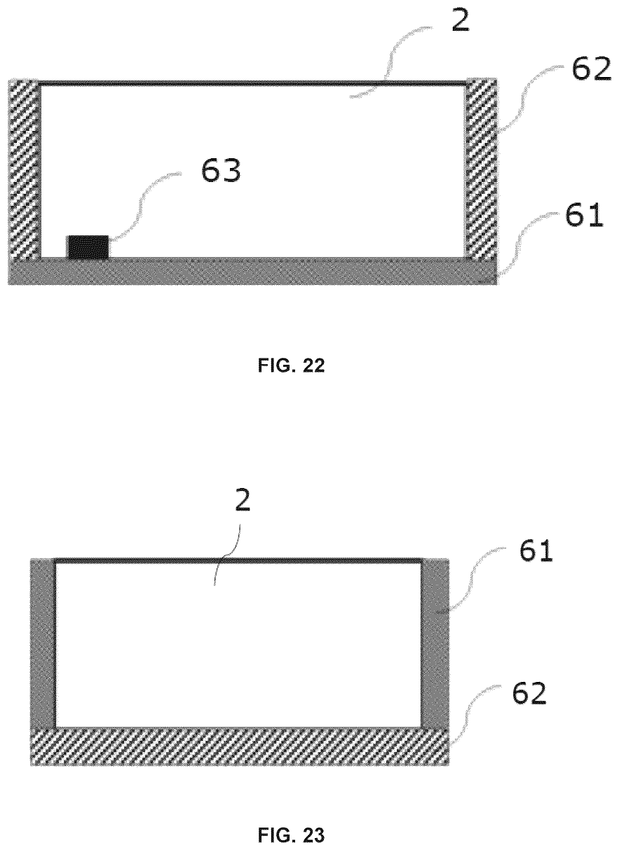

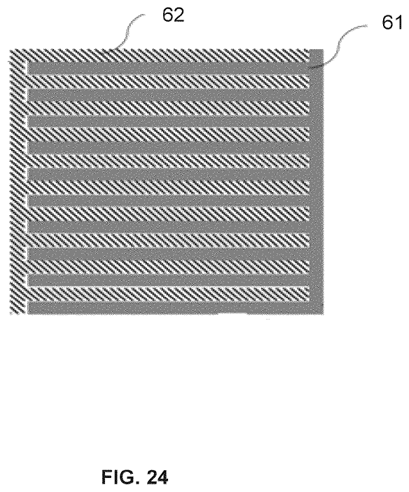

In some embodiments, referring to FIGS. 22-24, the heating assembly may include a plurality of heating sheets 61. In some embodiments, the refrigeration assembly may include a plurality of refrigeration sheets 62.

In some embodiments, the heating assembly and the refrigeration assembly may be provided on outer walls of the mixing chamber 101 and the culture chamber 2. In some embodiments, the heating assembly may include a plurality of heating sheets 61, which are provided on the outer wall of the culture chamber 2. The heating sheet may be at least one of a transparent heating plate, a metal heating plate, a semiconductor heating plate, a heating blanket, or a heating plate.

In some embodiments, the refrigeration sheets 62 may be provided on the outer wall of the culture chamber 2. The refrigeration sheet may be a semiconductor refrigeration sheet, or other refrigeration materials.

In some embodiments, the heating sheets 61 and the refrigeration sheets 62 may be located at different positions in the culture chamber 2. For example, the heating sheets 61 may be located at a bottom of the culture chamber 2 and the refrigeration sheets 62 may be located on a side of the culture chamber 2. As another example, the heating sheets 61 may be located on the side of the culture chamber 2 and the refrigeration sheets 62 may be located at the bottom of the culture chamber 2. As another example, the heating sheets 61 and the refrigeration sheets 62 may also be located at the same position in the culture chamber 2, e.g., on the same side or at the bottom of the culture chamber 2.

In some embodiments, energy of the plurality of heating sheets 61 may be delivered to the plurality of refrigeration sheets 62.

In some embodiments, the heating sheets 61 and the refrigeration sheets 62 may be disposed at intervals on the same side or at bottom of the culture chamber 2. Specifically, the plurality of heating sheets 61 may be disposed at intervals, the plurality of refrigeration sheets 62 may be disposed at intervals, one refrigeration sheet may be disposed between two adjacent heating sheets, and one heating sheet may be disposed between two adjacent refrigeration sheets. In some embodiments, the heating sheet may be in close contact with the two adjacent refrigeration sheets, and the refrigeration sheet 62 may be in close contact with the two adjacent heating sheets, which may enable the energy from the plurality of heating sheets 61 to be delivered to the plurality of refrigeration sheets 62.

In some embodiments, the heating sheets 61 and the refrigeration sheets 62 may be embedded in each other. Specifically, the heating sheets 61 and the refrigeration sheets 62 may be made of materials with good thermal conductivity, the structure of which is processed into an embeddable structure, causing the heating sheets 61 to be embedded between the refrigeration sheets 62, and the refrigeration sheets 62 to be embedded between the heating sheets 61, so as to realize the delivery of energy between the heating sheets and the refrigeration sheets.

In some embodiments, the heating sheets 61 and the refrigeration sheets 62 may work together to heat or cool the culture chamber 2. In some embodiments, when the culture chamber 2 needs to be heated up, the heating sheets 61 may be heated to a specified temperature under the control of the temperature control module, and since the refrigeration sheets 62 may be heat-conducting materials and the heat from the heating sheets 61 may be transferred to the refrigeration sheets 62, the refrigeration sheets 62 may be heated while heating the culture chamber 2, and the temperature of the refrigeration sheets 62 may quickly rise to the specified temperature, and then the culture chamber 2 may be heated by the heating sheets 61 and the refrigeration sheets 62 together. When the culture chamber 2 needs to be cooled down, the refrigeration sheets 62 may be cooled down to a specified temperature under the control of the temperature control module, and since the heating sheets 61 are heat-conducting materials and the heat of the heating sheets 61 are transferred to the refrigeration sheets 62, the heating sheets 61 may be cooled down while cooling down the culture chamber 2 by the refrigeration sheets 62, and the temperature of the heating sheets 61 may be quickly reduced to the specified temperature, and then the culture chamber 2 may be cooled down by the refrigeration sheets 62 and the heating sheets 61 together. The heating sheets 61 and the refrigeration sheets 62 may be staggered for transferring heat, causing the heating sheets 61 and the refrigeration sheets 62 may jointly heat and cool the culture chamber 2, increasing a heat exchange area, and improving the heat exchange efficiency.

In some embodiments, the refrigeration assembly may further include at least one cryogenic culture fluid device. The cryogenic culture fluid device may be used to introduce a cryogenic culture fluid into the chamber to cool the chamber to the first temperature. In some embodiments, the heating assembly may further include at least one thermal radiation emitting device. The thermal radiation emitting device may be used to emit thermal radiation into the chamber to heat the chamber to the second temperature.

In some embodiments, the cryogenic culture fluid device and the thermal radiation emitting device may be located at different positions in the culture chamber 2. For example, the cryogenic culture fluid device may be located inside the culture chamber 2 and the thermal radiation emitting device may be located on the side of the culture chamber 2. As another example, the cryogenic culture fluid device may be located at a top of the culture chamber 2 and the thermal radiation emitting device may be located at the bottom of the culture chamber 2. In some embodiments, the cryogenic culture fluid device and the thermal radiation emitting device may also be located at the same position in the culture chamber 2, e.g., on the same side or at top of the culture chamber 2.

In some embodiments, the temperature control unit 6 may further include a temperature detection member 63. The temperature detection member 63 may be a temperature sensor. The temperature detection member 63 may be electrically connected with the temperature control module, and the temperature detection member 63 may be provided on an inner wall of the culture chamber 2 or on the outer wall of the culture chamber 2.

In some embodiments, the temperature detection member 63 may be used to detect the temperature of the culture fluid in the culture chamber and feed a detected value back to the temperature control module, and the temperature control module may be configured to control the heating assembly to heat the culture chamber 2 or the refrigeration assembly to cool the culture chamber 2 by comparing the temperature detected by the temperature detection member 63 with an actual desired set value. In some embodiments, when the temperature detected by the temperature detection member 63 is lower than the actual desired set value, the temperature control module may control the heating assembly to heat the culture chamber 2 to the set value. In some embodiments, when the temperature detected by the temperature detection member 63 is higher than the actual desired set value, the temperature control module may control the heating assembly to cool the culture chamber 2 to the set value.

The structure and setting of the heating assembly, the refrigeration assembly, and the temperature detection member 63 on the mixing chamber 101 are the same as those of the culture chamber 2, which are not repeated herein.

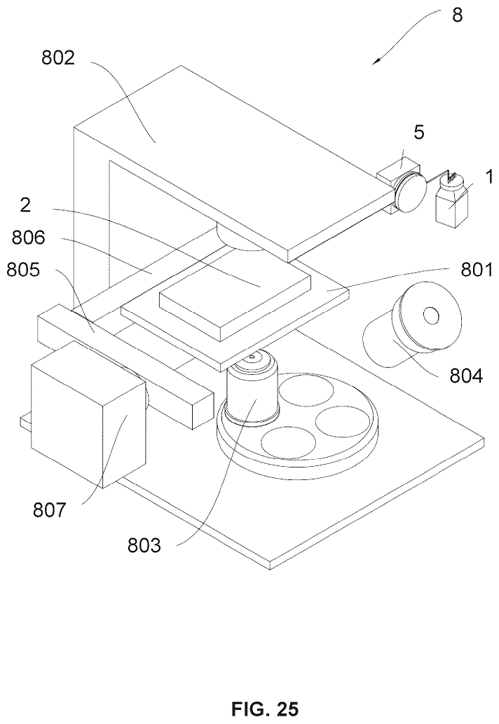

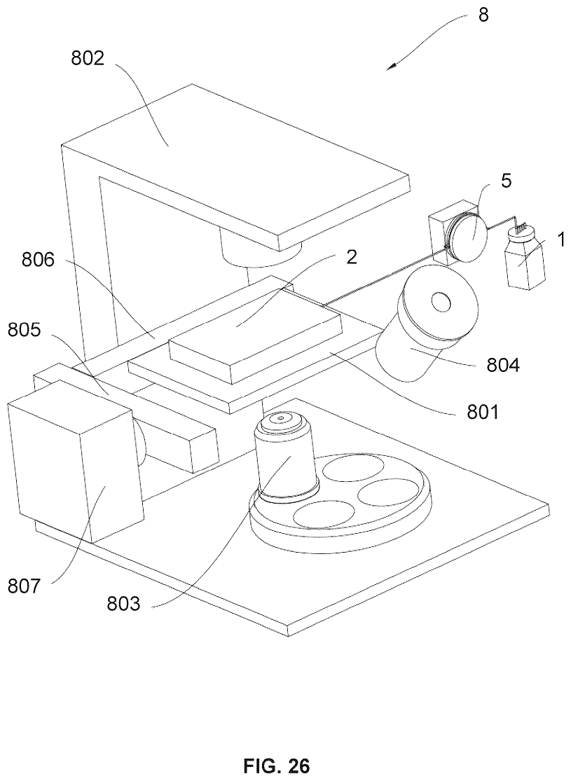

In some embodiments, referring to FIGS. 25-27, the culture module 110 may also include a microscopic observation module 8. The microscopic observation module 8 may observe and record the growth of the culture in the culture chamber 2. In some embodiments, according to the growth of the culture, the culture fluid provision module 120 and the fluid output module 130 may be operated to regulate the culture fluid in the culture chamber 2. In some embodiments, the microscopic observation module 8 may be fixedly disposed outside the culture chamber 2 for observing the culture chamber 2.

In some embodiments, the microscopic observation module 8 may include an observation assembly, the observation assembly being used to observe the culture in the culture chamber 2. In some embodiments, the microscopic observation module 8 may also include a stage 801 and a frame 802. The culture chamber 2 may be placed on the stage 801 to facilitate observation of the culture in the culture chamber 2 through the observation assembly. The frame 802 may be used to provide a platform for mounting and fixing the stage 801.

In some embodiments, the observation assembly may include an objective lens 803 and an eyepiece 804 through which an observer may observe the culture. In some embodiments, the objective lens 803 may be provided on the frame 802, which is capable of moving in a vertical direction. In some embodiments, the eyepiece 804 may also be provided on the frame 802, and the culture in the culture chamber 2 may be observed through the eyepiece 804.

The frame 802 may be provided with a first mechanical track 805, and a second mechanical track 806 may be slidably provided on the first mechanical track 805. In some embodiments, the first mechanical track 805 and the second mechanical track 806 may be provided perpendicular to each other and located in a horizontal plane. The stage 801 may be slidably provided on the second mechanical track 806, and adjustment of the stage 801 at any position in the horizontal plane may be achieved by sliding the stage 801 relative to the second mechanical track 806 and sliding the second mechanical track 806 relative to the first mechanical track 805, so as to facilitate observation of the culture chamber 2 directly by the objective lens 803, and observation of the culture using the eyepiece 804 to focus on the culture inside the culture chamber 2. If image data needs to be recorded during the observation process, a camera 807 may be used to take a picture to record the image data. In some embodiments, the culture chamber 2 may be shaken during the observation process so that the culture is in a better observation condition for the observation.

In some embodiments, the observation assembly may include an image acquisition device, and the image acquisition device may be used to obtain image data of the culture. The image acquisition device may mainly include the camera 807 (e.g., a CCD or a CMOS camera). If the image data needs to be recorded during the observation process, the camera 807 may be used to take a picture to record the image data.

In some embodiments, referring to FIGS. 2-12 and FIG. 29, the culture module 110 may also include a mixing module 7, which is used to shake the culture fluid in the culture chamber 2, thereby providing a full opportunity for exchange between the culture fluid and the culture and improving the absorption efficiency of the culture fluid by the culture.

In some embodiments, the mixing module 7 may include, but is not limited to, one or more of a shaking structure and a stirring structure. In some embodiments, the shaking structure may be a structure capable of shaking the culture chamber 2. In some embodiments, the stirring structure may be a structure capable of stirring the culture fluid in the culture chamber 2. In some embodiments, the mixing module may include a shaking structure. In some embodiments, the shaking structure may be connected with the culture chamber 2. In some embodiments, the shaking structure may be controlled manually. In some embodiments, the user may observe the growth of the culture in the culture chamber 2 through the observation assembly and manually control the shaking structure to shake the culture chamber 2 according to the growth of the culture.

In some embodiments, the shaking structure may also be controlled by automation. In some embodiments, the mixing module 7 may also include a drive member controlled by a processor, and the processor may control the drive member to drive the shaking structure to shake the culture chamber 2 based on input information (e.g., image data obtained by the observation assembly). In some embodiments, the processor may cause the culture chamber 2 to start shaking when the culture is growing inadequately based on an observation result. In some embodiments, inadequate growth of the culture may be that a portion or all of the culture does not reach a predetermined growth maturity. In some embodiments, the processor may control the shaking structure to continuously shake the culture chamber 2 throughout the cultivation process of the culture and adjust a rate and an amplitude of the shaking based on an observation result. In some embodiments, a shaking amplitude and rate of the shaking structure may be matched to the rate of delivery of the culture fluid into the culture chamber 2. A high culture delivery rate matching with a small amplitude of shaking promotes the opportunity for exchange of the culture fluid with the culture, but results in an excessive consumption of the culture fluid. A low culture fluid delivery rate matching with a large amplitude of mechanical shaking may simultaneously achieve the purpose of updating the culture fluid, increasing the opportunity for exchange of the nutrient solution with the culture, and facilitating the exchange of nutrient substances between the various chambers in the culture chamber 2.

In some embodiments, the shaking structure may include an oscillating shaker. In some embodiments, the oscillating shaker may include a stage 801 that places the culture chamber 2. In some embodiments, the culture chamber 2 may be located at a center of the oscillating shaker (e.g., the stage 801), and the width of the oscillating shaker matches with the width of the culture chamber 2. For example, the width of the oscillating shaker may be larger than or equal to the width of the culture chamber 2 to cause the culture chamber 2 being placed in the oscillating shaker. In some embodiments, the culture chamber 2 may move with the oscillating shaker after being placed in the oscillating shaker, i.e., there is no relative motion between the culture chamber 2 and the oscillating shaker. In some embodiments, the culture chamber 2 may be detachably fixed on the oscillating shaker. In some embodiments, a shaking mode of the shaker may be a pulse shaking (i.e., a periodic rapid shaking to a specified position over a short period of time). In some embodiments, the shaker oscillates at an inclination angle of 1° to 15°. In some embodiments, the shaker oscillates at an inclination angle of 1° to 10°. In some embodiments, the shaker oscillates at an inclination angle of 1° to 5°. In some embodiments, the shaker oscillates at an inclination angle of 5° to 15°. In some embodiments, the shaker oscillates at an inclination angle of 5° to 10°. In some embodiments, the shaking process of the shaker may be to first incline to one side to quickly reach a specified inclination angle and then return to a middle balance position after a certain time interval t1, and incline to the other side after a certain time interval t2, and return to the middle balance position after a certain time interval t3. In some embodiments, t1, t2, and t3 may be in a range of is to 1800s. In some embodiments, the smaller t1, t2, and t3, the higher the shaking frequency, indicating that it is more favorable for nutrient exchange. In some embodiments, at least one of t1, t2, or t3 may be in a range of is to 500s. In some embodiments, at least one of t1, t2, or t3 may be in a range of is to 300s. In some embodiments, at least one of t1, t2, or t3 may be in a range of 1 s to 200s. In some embodiments, at least one of t1, t2, or t3 may be in a range of is to 100s. In some embodiments, at least one of t1, t2, or t3 may be in a range of is to 80s. In some embodiments, at least one of t1, t2, or t3 may be in a range of is to 50s. In some embodiments, at least one of t1, t2, or t3 may be in a range of is to 10s. In some embodiments, the larger t1, t2, and t3, the lower the shaking frequency, indicating that it is the less damage to the cells. In some embodiments, at least one of t1, t2, or t3 may be in a range of 300s to 1800s. In some embodiments, at least one of t1, t2, or t3 may be in a range of 500 s to 1800s. In some embodiments, at least one of t1, t2, or t3 may be in a range of 1000 s to 1800s. In some embodiments, at least one of t1, t2, or t3 may be in a range of 1500 s to 1800s. In some embodiments, at least one of t1, t2, or t3 may be in a range of 1700 s to 1800s. In some embodiments, at least one of t1, t2, or t3 may be in a range of 1750 s to 1800s. In some embodiments, at least one of t1, t2, or t3 may be values that facilitating nutrient exchange and causing less damage to cells. In some embodiments, at least one of t1, t2, or t3 may be in a range of 200 s to 500s. In some embodiments, at least one of t1, t2, or t3 may be in a range of 200 s to 400s. In some embodiments, at least one of t1, t2, or t3 may be in a range of 250 s to 300s. In some embodiments, at least one of t1, t2, or t3 may be in a range of 300 s to 350s. In some embodiments, the greater oscillating amplitude of the shaker may be more favorable for nutrient exchange. In some embodiments, an inclination angle of the oscillating amplitude of the shaker may be in a range of 5° to 20°. In some embodiments, an inclination angle of the oscillating amplitude of the shaker may be in a range of 7° to 20°. In some embodiments, an inclination angle of the oscillating amplitude of the shaker may be in a range of 10° to 20°. In some embodiments, the oscillating amplitude of the shaker is smaller, indicating that it is less damage to the cells. In some embodiments, an inclination angle of the oscillating amplitude of the shaker may be in a range of 1° to 10°. In some embodiments, the inclination angle of the oscillating amplitude of the shaker may be in a range of 1° to 7°. In some embodiments, an inclination angle of the oscillating amplitude of the shaker may be in a range of 1° to 5°. In some embodiments, the oscillating amplitude of the shaker may be balanced to promote nutrient exchange and cause less damage to the cells. In some embodiments, an inclination angle of the oscillating amplitude of the shaker may be in a range of 5° to 10°. In some embodiments, the inclination angle of the oscillating amplitude of the shaker may be in a range of 6° to 8°. In some embodiments, an inclination angle of the oscillating amplitude of the shaker may be in a range of 6° to 7°. In some embodiments, an inclination angle of the oscillating amplitude of the shaker may be in a range of 7° to 8°.

In some embodiments, the shaking mode of the shaker may also be a continuous oscillation. In some embodiments, the shaker oscillates from a lowest position on one side to a lowest position on the other side and returns to the lowest position on a starting side to achieve the continuous oscillation. In some embodiments, the frequency of the continuous oscillation depends on an oscillation rate; the faster the oscillation rate, the shorter the time required for a single oscillation, and the higher the frequency. In some embodiments, an inclination angle of the continuous oscillating amplitude of the shaker may refer to the inclination angle of the oscillating amplitude of the oscillating shaker, which is not repeated herein. In some embodiments, a time of one oscillation of the continuous oscillation of the shaker may also refer to as a time of one oscillation of the oscillating shaker, which is not repeated herein.

In some embodiments, a motion mode of the shaker may also be circular motion, i.e., the shaker moves in a circular motion around a certain circumferential center. In other words, the shaking structure may also include a circular motion shaker. In some embodiments, the culture chamber 2 may move with the circular motion shaker after being placed in the circular motion shaker, i.e., there is no relative motion between the culture chamber 2 and the circular motion shaker. In some embodiments, the culture chamber 2 may be detachably fixed to the circular motion shaker.

In some embodiments, a shaking parameter of the circular motion shaker may include a circular motion amplitude and a shaking rate of the shaker. The circular motion amplitude of the shaker may be understood as a circular motion amplitude and a shaking rate of a central axis of the shaker. In some embodiments, the central axis of the shaker may be an axis perpendicular to a plane in which the shaker is located and overlapping through a geometric center of the shaker. In some embodiments, the central axis of the shaker may be moved in a circular motion around a circular center in the plane in which the shaker is located, thereby achieving the motion of the shaker. In some embodiments, the circular motion amplitude of the central axis of the shaker may be a distance from the circular center to the central axis of the shaker. In some embodiments, the shaking rate may be a rotational rate at which the central axis of the shaker performs a circular motion around the circular center.

In some embodiments, the circular motion amplitude of the central axis of the shaker may be in a range of 10 mm˜30 mm. In some embodiments, the circular motion amplitude of the central axis of the shaker may be in a range of 10 mm˜20 mm. In some embodiments, the circular motion amplitude of the central axis of the shaker may be in a range of 15 mm˜20 mm. In some embodiments, the circular motion amplitude of the central axis of the shaker may be in a range of 20 mm˜30 mm. In some embodiments, the circular motion amplitude of the central axis of the shaker may be in a range of 20 mm˜25 mm. In some embodiments, the circular motion amplitude of the central axis of the shaker may be in a range of 21 mm˜22 mm. In some embodiments, a shaking rate of the circular motion shaker may be in a range of 10 r/min˜300 r/min. In some embodiments, a shaking rate of the circular motion shaker may be in a range of 10 r/min˜200 r/min. In some embodiments, a shaking rate of the circular motion shaker may be in a range of 50 r/min˜200 r/min. In some embodiments, a shaking rate of the circular motion shaker may be in a range of 50 r/min˜100 r/min. In some embodiments, a shaking rate of the circular motion shaker may be in a range of 100 r/min˜150 r/min. In some embodiments, a shaking rate of the circular motion shaker may be in a range of 90 r/min˜100 r/min. In some embodiments, a shaking rate of the circular motion shaker may be in a range of 100 r/min˜110 r/min. In some embodiments, a shaking rate of the circular motion shaker may be in a range of 99 r/min˜101 r/min.

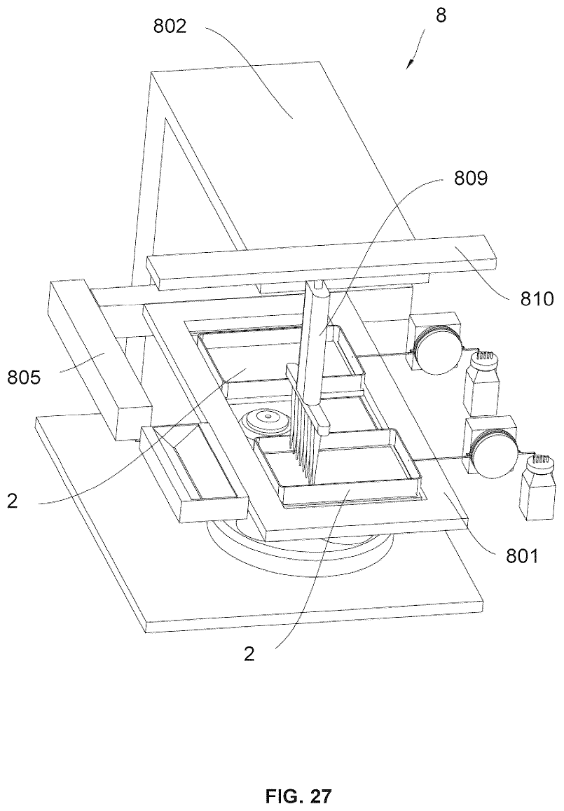

In some embodiments, the shaking structure may be placed on the stage 801 and the culture chamber 2 may be placed on the shaking structure. In some embodiments, the stage 801 may be provided with a placement station, and the shaking structure may be placed on the placement station. In other embodiments, the stage 801 may be provided with multiple placement stations (specifically refer to FIG. 27), and a shaking structure may be placed on each placement station.

In some embodiments, the mixing module 7 may include a stirring structure. In some embodiments, the stirring structure may include one or more stirring modes. In some embodiments, the stirring mode may include, but is not limited to, a mechanical stirring. In some embodiments, the mechanical stirring structure may include a mechanical stirring assembly, the mechanical stirring assembly being capable of stirring the culture fluid in the culture chamber 2, thereby improving the opportunity for exchange of the culture fluid with the culture. In some embodiments, the mechanical stirring assembly may be mounted in the culture chamber 2. In some embodiments, the mechanical stirring assembly may also be mounted outside the culture chamber 2 and be capable of extending into the culture chamber 2 from outside the culture chamber 2 to stir the culture fluid in the culture chamber 2.

In some embodiments, the mechanical stirring assembly may include a stirring member. The mechanical stirring assembly may stir the culture fluid by a circular motion of the stirring member. The stirring rate of the mechanical stirring assembly refers to a stirring rate of the stirring member, i.e., a rotation rate of the stirring member performing a circular motion. In some embodiments, a stirring rate of the mechanical stirring assembly may be no higher than 500 r/min. In some embodiments, the stirring rate of the mechanical stirring assembly may be no higher than 400 r/min. In some embodiments, the stirring rate of the mechanical stirring assembly may be no higher than 300 r/min. In some embodiments, the stirring rate of the mechanical stirring assembly may be no higher than 200 r/min. In some embodiments, the stirring rate of the mechanical stirring assembly may be no higher than 100 r/min. In some embodiments, the stirring rate of the mechanical stirring assembly may be in a range of 80 r/min-100 r/min. In some embodiments, the stirring rate of the mechanical stirring assembly may be in a range of 90 r/min-100 r/min. In some embodiments, the stirring rate of the mechanical stirring assembly may be in a range of 95 r/min-105 r/min. In some embodiments, the stirring rate of the mechanical stirring assembly may be in a range of 100 r/min-105 r/min. In some embodiments, the stirring rate of the mechanical stirring assembly may be lower, which may stir the culture to suspend in the culture fluid. In some embodiments, the stirring rate of the mechanical stirring assembly may be higher, which may make the contact between the culture and the culture fluid more adequate.

In some embodiments, the mechanical stirring assembly may stir the culture fluid by reciprocating linear motion of the stirring member. In some embodiments, a frequency of the reciprocating linear motion of the stirring member of the mechanical stirring assembly may be in a range of 20 times/min-100 times/min, a round trip of the reciprocating linear motion completed by the mechanical stirring assembly being 1 time.

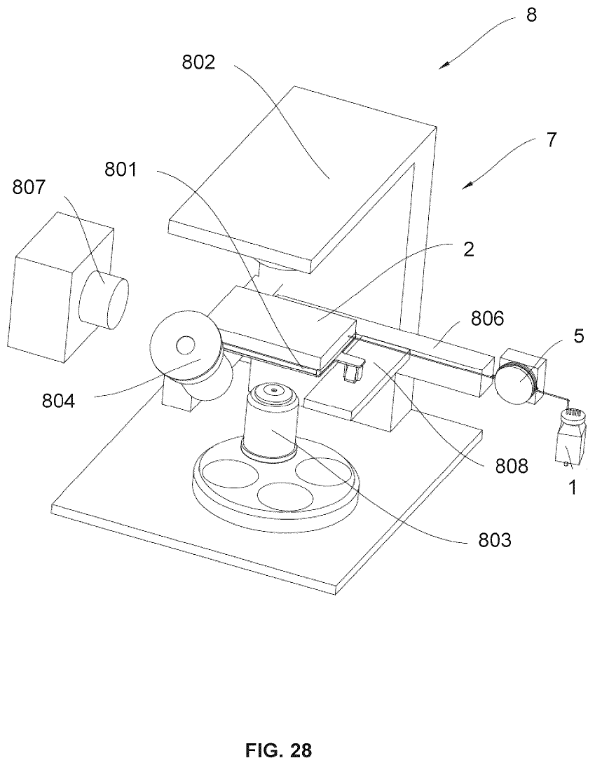

In some embodiments, the mixing module 7 may also be a portion of the structure in the microscopic observation module 8. Referring to FIG. 28, in some embodiments, the mixing module 7 may include a stage 801 and a frame 802, the culture chamber 2 may be placed on the stage 801, and the stage 801 may be capable of driving the culture chamber 2 to shake. The frame 802 may be used to provide a platform for mounting and fixing the stage 801. The stage 801 may be capable of holding at least one culture chamber 2. In some embodiments, a placement station may be provided on the stage 801, and the culture chamber 2 may be placed on the placement station. In other embodiments, the stage 801 may be provided with a plurality of placement stations, each of which may hold a culture chamber 2.

In some embodiments, the mixing module 7 and the temperature control unit 6 may be mutually independent structural modules. In some embodiments, the mixing module 7 and the temperature control unit 6 may also be integrated into one structural module, and the integrated structural module may be a mixing temperature control module, which is used to control the temperature of the culture chamber 2 and shake the culture fluid inside the culture chamber 2. The mixing temperature control module may include a mechanical shaking mechanism and a temperature control assembly, the mechanical shaking mechanism may be provided with a mounting surface for placing the culture chamber 2, so that the culture chamber 2 is fixed relative to the mechanical shaking mechanism after being mounted on the mounting surface, and the culture chamber 2 may be driven to shake by shaking the mounting surface. In some embodiments, the culture chamber 2 may also be detached from the mounting surface. In some embodiments, the mounting surface may be also provided with a temperature control assembly. In some embodiments, the mounting surface may be a plane of the mechanical shaking structure, an upper surface of the plane being provided with the temperature control assembly, and when the culture chamber 2 is mounted on the mounting surface, a bottom surface of the culture chamber 2 may be in contact with the temperature control assembly on the upper surface of the mounting surface. In some embodiments, the mounting surface may be a plurality of planes of the mechanical shaking structure, and one or more planes of the mounting surface may be provided with the temperature control assembly, and when the culture chamber 2 is mounted on the mounting surface, the bottom surface or a side surface of the culture chamber 2 may be in contact with the temperature control assembly on the one or more planes of the mounting surface. The temperature control assembly may be used to control the temperature of the culture chamber 2, specifically, the temperature control assembly may heat the mixing chamber 101 and the culture chamber 2, so that the temperature of the culture fluid in the mixing chamber 101 and the culture fluid in the culture chamber 2 raises to a physiological temperature suitable for the growth of the culture to cultivate the culture. After the cultivation is completed, a matrix gel is required to be removed, and the culture is collected, the temperature control assembly may cause the temperature of the culture fluid in the culture chamber 2 to drop to a temperature at which the matrix gel in the culture fluid is liquefied to collect the culture. Specifically, the temperature control assembly may include a heating assembly, a refrigeration assembly, and a temperature control module, the heating assembly and the refrigeration assembly may be electrically connected with the temperature control module, and the temperature control module may be used to control the heating assembly to heat the culture chamber 2, and control the refrigeration assembly to cool the culture chamber 2.

In some embodiments, the mixing module 7 may further include a drive member. In some embodiments, the drive member may include, but is not limited to, a linear motor, a rotary motor, a cam motor, etc. In some embodiments, the drive member may include an output end. In some embodiments, a motion form output by the output end may include, but is not limited to, a linear motion, a rotary motion, a circular motion, etc. In some embodiments, the output end may be connected with the shaking structure for driving the shaking structure to shake the culture fluid in the culture chamber 2 to provide a fuller exchange of the culture fluid and the culture. In some embodiments, the output end may be connected with the stage 801. In some embodiments, the output end may be connected with the mechanical mixing assembly (e.g., a stirring member) for driving the mechanical mixing assembly to move.

In some embodiments, the processor may obtain image data recorded by the camera 807 from the observation assembly. In some embodiments, the image data recorded by the camera 807 may contain type information of a culture, a cultivation time of a culture, and a cultivation degree of a culture, etc. In some embodiments, the type information of the culture, the cultivation time of the culture, and the cultivation degree of the culture may be information inputted humanly by an operator before the camera 807 captures an image of the culture or after the camera 807 captures an image of the culture. In some embodiments, the type information of the culture, the cultivation time of the culture, and the cultivation degree of the culture may also be information that the processor automatically obtains from a preset image library after the camera 807 takes an image of the culture. In some embodiments, the preset image library may be stored in a storage device that may interact data with the processor. In some embodiments, the preset image library may include images that have type information of the different culture, the cultivation time of the different culture, and the cultivation degree of the different culture, and the processor may determine a preset image that is closest to the currently captured image through an image comparison algorithm, and add the type information of the culture, the cultivation time of the culture, and the cultivation degree of the culture of the preset image to the currently captured image data.

In some embodiments, the processor may control the drive member based on the image data of the culture. In some embodiments, the processor may control the drive member to drive the stage 801 to shake based on the image data. In some embodiments, the processor may include a trained machine learning model. In some embodiments, the machine learning model may include, but is not limited to, a convolutional neural network (CNN) model, a recurrent neural network (RNN) model. In some embodiments, the processor may input the image data into the machine learning model, and the machine learning model may output a corresponding control parameter for the drive mechanism. In some embodiments, the control parameter may be a shaking parameter (e.g., an amplitude, a time, a frequency of the shaking, etc.) or several preset shaking modes. In some embodiments, the machine learning model may be obtained by training.

In some embodiments, an input to the machine learning model may be a sample image of the culture. In some embodiments, the sample image of the culture may include type information of a culture, a cultivation time of a culture, and a cultivation degree of a culture, etc. In some embodiments, a training label of the machine learning model may include a control parameter corresponding to each of the image data in the sample image. In some embodiments, the control parameter may include a preset shaking mode. In some embodiments, the control parameter may also include a shaking amplitude, a shaking rate, a shaking frequency, and a shaking time. In some embodiments, a sample image of a culture with a long cultivation time and a low cultivation degree corresponds to a violent shaking mode; and a sample image of a culture with a high cultivation degree corresponds to a mild shaking mode. In some embodiments, a sample image of a culture with a long cultivation time and a low cultivation degree may correspond to a large shaking amplitude (e.g., an edge of the culture chamber 2 deviating outwardly from its original position by 5 cm). In some embodiments, the sample image may be input to an initial machine learning model, and the initial machine learning model may be trained with a control parameter corresponding to the sample image as a training label, and a trained machine learning model may be obtained.

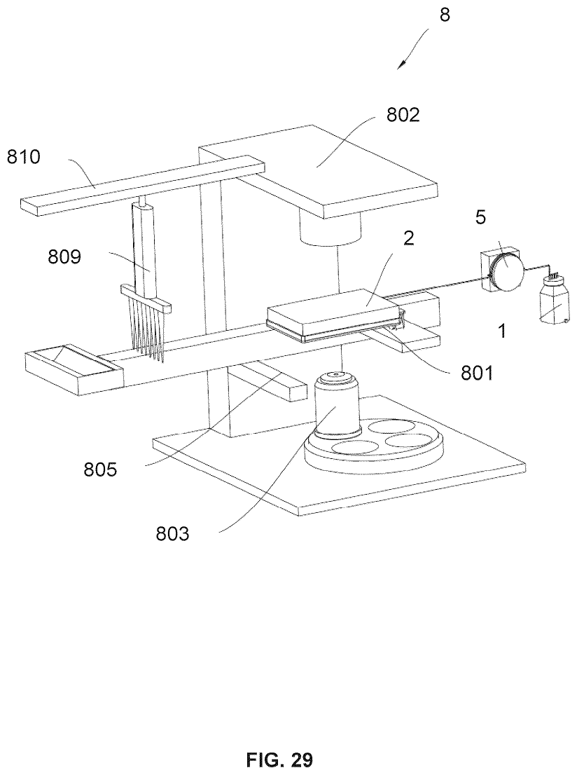

In some embodiments, referring to FIG. 29, the microscopic observation module 8 may further include an automatic feeding module, the automatic feeding module being used to automatically add a culture to the culture chamber 2.

In some embodiments, the automatic feeding module may include an automatic feeder 809 and an automatic feeding track 810. In some embodiments, the automatic feeding track 810 may be provided on the frame 802, the automatic feeder 809 may be slidably provided on the automatic feeding track 810, and the automatic feeder 809 may be driven by a feeding drive member to slide on the automatic feeding track 810 to perform feeding. The automatic feeder 809 may be mainly used to add a sample using the automatic feeder 809 when it is necessary to add a culture, a reagent or a drug, etc. in the culture chamber 2, avoiding manual addition of the sample, ensuring hygienic conditions in the culture chamber 2, and making the culture less prone to contamination. In some embodiments, an operator may place a prepared cell sample in a feeding slot, and the automatic feeder 809 may move along the automatic feeding track 810 to a position of the feeding slot, take a certain amount of the cell sample, and then move to the position of the culture chamber 2, add the cell sample to the culture chamber 2, and complete the feeding of the sample.

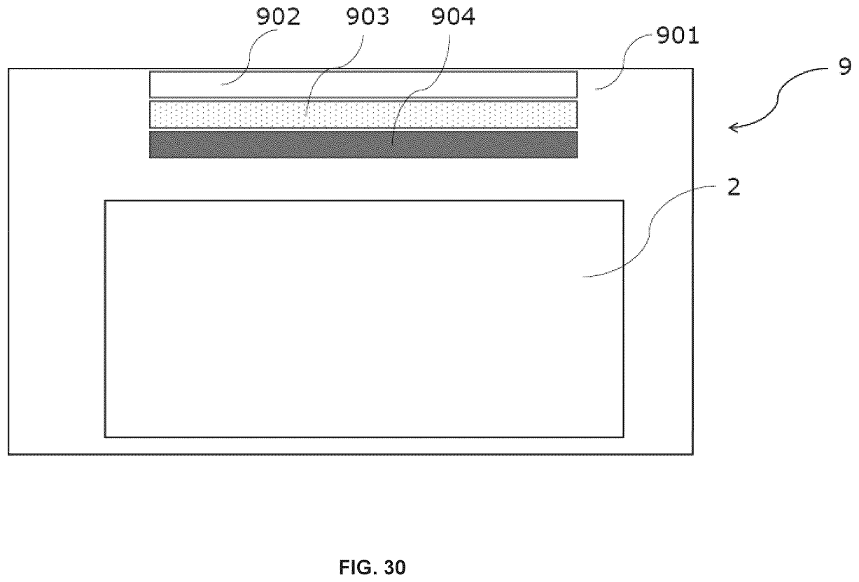

In some embodiments, referring to FIG. 30, the in vitro life culture system 100 may further include a sterility control module 9, which includes a sterile work chamber 901, a filtration assembly 903, and a sterilization assembly 904. In some embodiments, the sterile work chamber 901 may be used to keep the culture chamber 2 in a sterile culture environment. In some embodiments, the culture chamber 2 may be at least provided in the sterile work chamber 901. In some embodiments, the culture module 110, the culture fluid provision module 120, and the fluid output module 130, etc. may be all provided in the sterile work chamber 901, i.e., all parts of the culture system 100 of the culture, except for the sterile control module 9, may be located in the sterile work chamber 901.

The sterile work chamber 901 may be provided with a vent, the filtration assembly 903 may be provided at the vent, and an air intake fan 902 may be provided on the vent, through which gas may be introduced into the sterile work chamber 901. The filtration assembly 903 may be used to filter particles in the gas introduced into the sterile work chamber 901, causing that the particles such as dust, impurities, etc. cannot enter the sterile work chamber 901. The sterilization assembly 904 may be used to sterilize and disinfect the sterile work chamber 901. Specifically, the sterilization assembly 904 may be an ultraviolet lamp, which is provided at the air inlet, and the sterilization assembly 904 may sterilize both the sterile work chamber 901, and also sterilize the gas introducing into the sterile work chamber 901, which may prevent the gas with bacteria from entering into the sterile work chamber 901.

In some embodiments, at least one of the culture module 110, the culture fluid provision module 120, the fluid output module 130, and the culture fluid circulation module 140 may be a disposable consumable. The disposable consumable means materials disposed of as waste after being used once, which ensures a hygienic condition during the perfusion cultivation process, and avoids affecting the normal perfusion cultivation due to the contamination of the culture module 110, the culture fluid provision module 120, the fluid output module 130, and the culture fluid circulation module 140. In some embodiments, the disposable consumable may include a culture medium disposable, a glass disposable, a plastic disposable, etc. In some embodiments, at least one vessel (e.g., the culture chamber 2) in the culture module 110 for holding the culture fluid or culture may be a disposable consumable. In some embodiments, at least one vessel holding the culture fluid or gas (e.g., the mixing chamber 101) in the culture fluid provision module 120 may be a disposable consumable. In some embodiments, at least one vessel holding the culture fluid (e.g., the collection unit 4) in the fluid output module 130 may be a disposable consumable. In some embodiments, at least one vessel holding the culture fluid in the culture fluid circulation module 140 (e.g., the exchange unit 3) may be a disposable consumable.

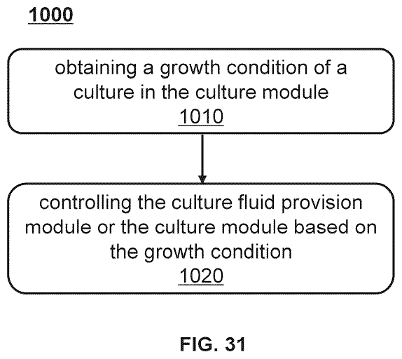

In some embodiments, the method for controlling an in vitro life culture system 100 may be applied to the in vitro life system 100 described above. In some embodiments, the method for controlling the in vitro life culture system 100 may be performed by a processor of the in vitro life system 100. In some embodiments, referring to FIG. 31, the method for controlling the in vitro life culture system 100 may include a process 1000, and the process 1000 may include following operations.

In 1010, obtaining a growth condition of a culture in the culture module.

In some embodiments, the processor may obtain the growth of the culture in the culture module 110. In some embodiments, the processor may determine the growth of the culture from information input by the operator. In some embodiments, the processor may obtain an image of the culture via the microscopic observation module 8 and determine the growth of the culture based on the obtained image and a preset algorithm.

In some embodiments, the processor may perform a geometric correction, size adjustment, and grayscale processing on the obtained image of the culture. In some embodiments, the processor may also perform a Gaussian filtering process on the obtained image of the culture to eliminate and suppress noise. In some embodiments, the processor may also perform a MorpHology morphological transformation process on the obtained image of the culture to further eliminate noise.

In some embodiments, the stage 801 may be placed obliquely, resulting in the obtained image being in a deflected position, and the processor may rotate the stage 801 to correct when obtaining an image region, thereby improving the positioning accuracy of the obtained image.

In some embodiments, under different lighting conditions, a color of the culture changes, and an electronic device cannot self-correct according to the change of light, thus the obtained image may appear to be color distorted, reddish or bluish. The processor may perform a white balance process on the obtained image. The white balance is a main manner for correcting color cast images, thus improving the accuracy of the color of the obtained image. In some embodiments, the processor may obtain an image of the culture in the culture module 110 in real time via the microscopic observation module 8. In some embodiments, the image obtained by the processor may be imported into a computer or other device with a display function for display.

In some embodiments, the preset algorithm may include an image comparison algorithm, and the processor may determine the growth of the culture by the image comparison algorithm. In some embodiments, the in vitro life system 100 may include a storage device capable of data interaction with the processor, the storage device storing a preset image library. In some embodiments, the preset image library may be stored with preset images corresponding to types of different cultures, and growth of the different cultures. In some embodiments, the processor may determine a preset image that is closest to the currently obtained image through an image comparison algorithm, and obtain type information of culture, the growth of the culture, etc., from the preset image.

In some embodiments, the preset algorithm may include a machine learning model. In some embodiments, the machine learning model may be a convolutional neural network model. In some embodiments, the processor may perform an image recognition on the image of the culture by the machine learning model to determine the growth of the culture. In some embodiments, the growth of the culture may include a growth time of the culture, a growth degree of the culture, a growth stage of the culture, a growth rate of the culture, a growth stability of the culture, etc. In some embodiments, the growth time of the culture may be a total length of time of cultivating the culture when the image of the culture is taken. In some embodiments, the growth degree of the culture may be whether the culture grows well. In some embodiments, the growth stage of the culture may be a stage differentiated by the maturity of the culture in the growth and development. In some embodiments, the growth rate of the culture may be a rate of growth of the culture. In some embodiments, the growth stability of the culture may be a rate at which the growth degree of the culture changes over time, when the growth degree of the culture changes slowly over time, the growth stability of the culture may be high, and when the growth degree of the culture changes fast over time, the growth stability of the culture may be low.

In some embodiments, a training process of the machine learning model may include: using images of cultures with different growth conditions as sample images, and labelling the sample images. In some embodiments, the sample images may be labelled as “Stage 1” and “Stage 2” according to different stages of the growth conditions. In some embodiments, the sample images may be labelled as “good” and “poor” according to the growth degree of the growth conditions. In some embodiments, the sample images may be labeled as “stable” and “unstable” according to the growth stability of the growth conditions. In some embodiments, the sample images may be labelled as “faster growing” and “slower growing” according to the growth rate of the growth conditions. The sample images and labels may be input to an initial machine learning model for training, the initial machine learning model may be evaluated based on a loss value and an accuracy rate, if a stable loss value and a stable accuracy rate are obtained, then the training may be terminated, otherwise the training and model evaluation process is repeated until the termination condition is met and a trained machine learning model is obtained.

In 1020, controlling the culture fluid provision module or the culture module based on the growth condition.

In some embodiments, the processor may control the culture fluid provision module 120 and the culture module 110 based on the growth conditions. In some embodiments, the processor may control the culture fluid provision module 120 or the culture module 110 based on different growth conditions of the culture.

In some embodiments, the culture module 110 may include an oscillating drive mechanism. The oscillating drive mechanism may be used to drive the mixing module 7 to shake the culture liquid in the culture chamber 2, thereby driving the culture liquid to exchange with the culture. In some embodiments, the oscillating drive mechanism may control a shaking mode of the mixing module 7, such as lighter shaking, more violent shaking. In some embodiments, the oscillating drive mechanism may also control a shaking parameter of the mixing module 7, including but not limited to at least one of a shaking amplitude, a shaking rate, a shaking frequency, and a shaking time. In some embodiments, the oscillating drive mechanism may be a positive drive, i.e., accelerating the mixing module 7 to shake the culture fluid in the culture chamber 2. In some embodiments, the oscillating drive mechanism may also be negative drive, i.e., slowing down the mixing module 7 to shake the culture fluid in the culture chamber 2.

In some embodiments, the processor may control a motion of the oscillating drive mechanism based on the growth conditions. In some embodiments, the processor may control the oscillating drive mechanism to accelerate the motion when the growth condition is poor, such as controlling the oscillating drive mechanism to speed up the shaking rate of the mixing module 7, increase the shaking amplitude of the mixing module 7, lengthen the shaking time and frequency of the mixing module 7 of the mixing module 7, etc.; and the processor may control the oscillating drive mechanism to slow down the motion when the growth condition exceeds a preset condition, such as controlling the oscillating drive mechanism to slow down the shaking rate of the mixing module 7, reduce the shaking amplitude of the mixing module 7, reduce the shaking time of the mixing module 7 and frequency of the mixing module 7, etc.

In some embodiments, the processor may control a rate at which the culture fluid is provided by the culture fluid provision module 120 based on the growth condition. In some embodiments, the processor may control the power unit 5 to control the delivery rate of the nutrient solution based on the growth condition, i.e., the flow rate of the nutrient solution flowing from the fluid replenishment chamber 104 into the mixing chamber 101. In some embodiments, when the growth condition is poor, the processor may control the culture fluid provision module 120 to increase the rate of providing the culture fluid, such as increasing a fluid flow rate or increasing a delivery power; when the growth condition exceeds the preset condition, the processor may control the culture fluid provision module 120 to slow down the rate of providing the culture fluid, such as reducing the fluid flow rate or attenuating the delivery power. In some embodiments, the processor may control the rate of providing the culture fluid at each interface by the culture fluid provision module 120 in real time based on the growth condition.

In some embodiments, during a static cultivation phase, the processor may control the rate of gas introduced from the gas mixing control unit 105 to the culture chamber 2 based on the growth condition. In some embodiments, the processor may control the power unit 5 to control a flow rate of the gas in a gas delivery pipeline 1011 based on the growth condition. In some embodiments, when the growth condition is poor due to lack of oxygen, the processor may control the power unit 5 to speed up the gas delivery rate for increasing the oxygen content of the culture fluid in the culture chamber 2. In some embodiments, the processor may also close a valve between the gas delivery pipeline 1011 and the culture chamber 2 based on good growth condition, so that the culture in the culture chamber 2 maintains in the current culture environment.

In some embodiments, the culture module 110 may include a temperature control unit. The temperature control unit may be used to control the temperature of the culture, e.g., raising or reducing the temperature of the culture. In some embodiments, the processor may control the temperature control unit to heat or cool the culture based on the growth condition. In some embodiments, the processor may control the temperature control unit to heat the culture to a certain temperature when the growth condition is poor; the processor may control the temperature control unit to cool the culture to a certain temperature when the growth condition exceeds the preset condition. In some embodiments, the temperature control unit may control the temperature of the culture to the first temperature after the culture is completed.

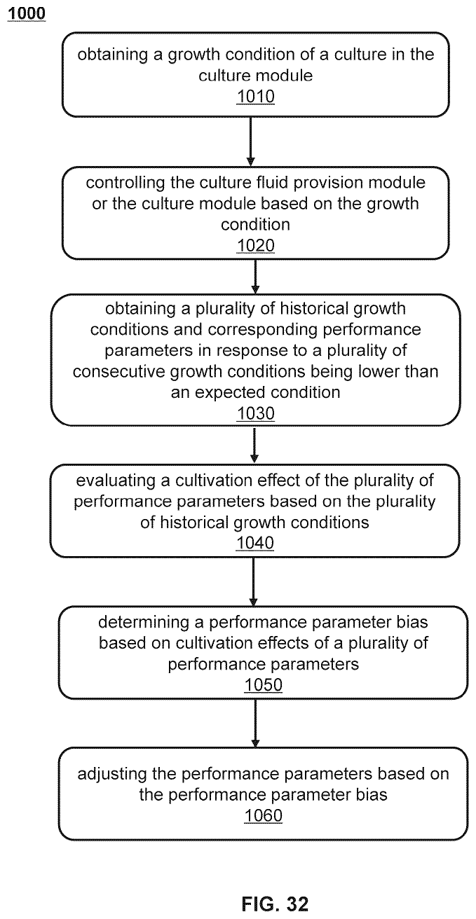

FIG. 32 is a flowchart illustrating a process for controlling a culture fluid provision module and/or a culture module based on a growth condition according to some embodiments of the present disclosure. As shown in FIG. 32, in some embodiments, the process 1000 may further include the following operations.

In 1030, obtaining a plurality of historical growth conditions and corresponding performance parameters in response to a plurality of consecutive growth conditions being lower than an expected condition.

In some embodiments, the processor may obtain a plurality of historical growth conditions and corresponding performance parameters when a plurality of consecutive growth conditions are lower than the expected condition. The growth conditions may be obtained based on operation 1010. The expected condition may be an expected growth time, an expected growth degree, an expected growth stage, an expected growth rate, an expected growth stability, etc. corresponding to the culture for a current cultivation time under a cultivation condition of a current performance parameter. The cultivation time may be determined based on the time when the operator inputs information and/or a time when an image of the culture is obtained. In some embodiments, for different cultures, the expected growth conditions may be different under the same cultivation time. In some embodiments, for the same culture, the expected growth conditions may vary accordingly with the cultivation time. It should be noted that the cultivation time of the culture is not equivalent to the growth time during the cultivation period. For example, a culture may not grow at the beginning of the cultivation of the culture, but start to grow after a period of cultivation time.

In some embodiments, the plurality of historical growth conditions refer to growth conditions of the culture before and after a plurality of recent adjustments of performance parameters prior to a current time point. Exemplarily, when the performance parameters are adjusted for n times, the plurality of historical growth conditions may include two historical growth conditions before and after a first adjustment of the performance parameters, two historical growth conditions before and after a second adjustment of the performance parameters, two historical growth conditions before and after an nth adjustment of the performance parameters. Only one historical growth condition may be obtained between the first adjustment and the second adjustment, between the second adjustment and the third adjustment, and between the (n−1)th adjustment and the nth adjustment, respectively. In some embodiments, the historical growth condition before and after the ith (i being a natural number between 1 and n) adjustment of the performance parameters may correspond to a time point, respectively, and a time interval between one of the above two time points and the time point of the ith adjustment of the performance parameters may be equal to a time interval between the other of the above two time points and the time point of the ith adjustment of the performance parameters. The performance parameters corresponding to the plurality of historical growth conditions are performance parameters after each adjustment. In some embodiments, the performance parameters may include at least one of a movement parameter of the oscillating drive mechanism of the culture module 110, a culture fluid provision rate of the culture fluid provision module 120, a control parameter of a drive member in the mixing module 7 for driving the shaking of the stage 801, a temperature parameter of the temperature control unit 6 of the culture module 110, a rate at which the gas is passed through the gas mixing control unit 105, etc. In some embodiments, the performance parameters may be characterized as a sequence of parameters, and each element in the sequence corresponding to a performance parameter.

In 1040, evaluating a cultivation effect of the plurality of performance parameters based on the plurality of historical growth conditions.

In some embodiments, the processor may determine a cultivation effect of corresponding performance parameters based on the plurality of historical growth conditions. Specifically, after the performance parameters are adjusted for the ith (i being a natural number between 1 and n) adjustment, the adjusted historical growth condition is subtracted from a pre-adjustment historical growth condition to obtain a result, and if the result is negative, it indicates that the cultivation effect of the performance parameters after the ith adjustment is poor; if the result is positive, it indicates that the cultivation effect of the performance parameters after for the ith adjustment is good.

In 1050, determining a performance parameter bias based on cultivation effects of a plurality of performance parameters.

In some embodiments, the processor may determine a performance parameter bias based on the cultivation effects of the plurality of performance parameters. If the cultivation effects are good, it indicates that the performance parameters are too large; and if the cultivation effects are poor, it indicates the performance parameters are too small. In some embodiments, the processor may determine the performance parameter bias and a probability of the performance parameter bias based on the cultivation effects of the plurality of performance parameters. In some embodiments, a probability of good cultivation effect or poor cultivation effect may be determined by a total count of the performance parameters corresponding to the cultivation effects, thus a probability of the performance parameters being too large or small is determined. Exemplarily, among eleven historical growth conditions, there are ten adjusted performance parameters, a count of performance parameters corresponding to good cultivation effects is six, and a ratio of the count of the performance parameters corresponding to good cultivation effects to ten adjusted performance parameters is 0.6; a count of the performance parameters corresponding to poor cultivation effects is four, and a ratio of the count of the performance parameters corresponding to poor cultivation effects to ten adjusted performance parameters is 0.4. Accordingly, the probability of the performance parameters being too large is 0.6, and the probability of the performance parameters being too small is 0.4.

In some embodiments, the performance parameter bias (or referred to as a bias of the performance parameters) may be determined by a parameter judgment model. The parameter judgment model may process inputs of the plurality of historical growth conditions, the corresponding plurality of cultivation effects, and the corresponding performance parameters to output the bias and probability of each parameter of the corresponding performance parameter. It should be noted that since the plurality of historical growth conditions are obtained under a condition that a plurality of consecutive growth conditions are lower than the expected condition, the plurality of historical growth conditions are lower than the expected condition. In some embodiments, the output of the parameter judgment model may be a performance parameter bias with a higher probability and its corresponding probability; when the probabilities are the same, any one of them may be output. Exemplarily, when the probability of the performance parameter being too large is 0.6 and the probability of the performance parameter being too small is 0.4, the output of the parameter judgment model is “too large, 0.6”. In some embodiments, the parameter judgment model may include, but is not limited to, a support vector machine model, a decision tree model, a neural network model, etc.

In some embodiments, the parameter judgment model may be obtained by training. Training samples may be a plurality of historical growth conditions, a corresponding plurality of cultivation effects, and corresponding performance parameters, and labels of the training samples may be a bias and a probability of the corresponding performance parameter. The training samples with the labels are input into an initial parameter judgment model, parameters of the initial parameter judgment model are updated by training, and when the trained model satisfies a preset condition, the training is completed and the trained parameter judgment model is obtained. In some embodiments, the parameter judgment model may also output main parameters of the performance parameters that affect the cultivation effect. Specifically, the main parameters in the performance parameters that affect the cultivation effect may be determined by analyzing a plurality of historical growth conditions, so as to add the main parameters to the labels of the training samples for training the parameter judgment model.

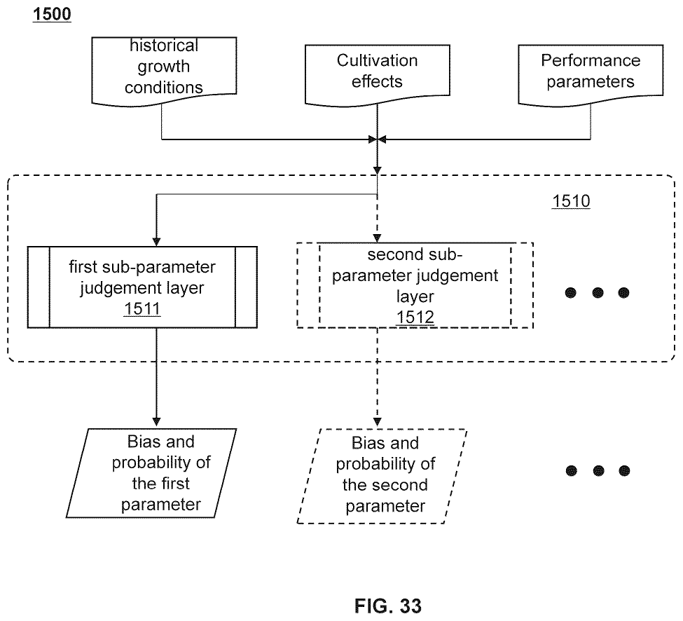

FIG. 33 is a schematic diagram illustrating a parameter judgment model according to some embodiments of the present disclosure.

As shown in FIG. 33, the parameter judgment model 1500 may include a parameter judgement layer 1510, and the parameter judgement layer 1510 may process the plurality of historical growth conditions, the corresponding plurality of cultivation effects, and the corresponding performance parameters to output a bias and a probability of the corresponding performance parameters. In some embodiments, the performance parameters may include at least a first parameter, and the parameter judgement layer 1510 may include at least a first sub-parameter judgement layer 1511, and the first sub-parameter judgement layer 1511 processes the plurality of historical growth conditions, the corresponding plurality of cultivation effects, and the corresponding first parameter to output the bias and probability of the first parameter. In some embodiments, the performance parameters may further include a second parameter, and correspondingly, the parameter judgement layer 1510 may include a second sub-parameter judgement layer 1512. The second sub-parameter judgement layer 1512 may process the plurality of historical growth conditions, the corresponding plurality of cultivation effects, and the corresponding second parameter to output the bias and probability of the second parameter. In some embodiments, the performance parameters may include a plurality of parameters, and the parameter judgement layer 1510 may include sub-parameter judgement layers that correspond one-to-one to the aforementioned plurality of parameters. Inputs of each sub-parameter judgement layer may be historical growth conditions, cultivation effects, and parameters corresponding to each sub-parameter judgement layer, and outputs of each sub-parameter judgement layer are bias and probability of the parameters corresponding to each sub-parameter judgement layer. In some embodiments, each sub-parameter judgement layer of the parameter judgement layer 1510 may be a neural network model. In some embodiments, the parameter judgment model 1500 may be obtained by a joint training of the respective sub-parameter judgement layer of the parameter judgement layer 1510. Training samples may include a plurality of historical growth situations, a corresponding plurality of cultivation effects, and a parameter of the performance parameter that corresponds to each of the sub-parameter judgement layers, and the labels of the training samples are the bias and probability of the parameter that corresponds to each of the sub-parameter judgement layers. The plurality of historical growth conditions and the corresponding plurality of cultivation effects in the training samples are input into the respective sub-parameter judgement layer of the parameter judgement layer 1510, and each parameter in the performance parameters in the training samples is input into the corresponding sub-parameter judgement layer respectively, a loss function is constructed based on the outputs of each sub-parameter judgement layer and labels, and the parameters of each sub-parameter judgement layer in the parameter judgment model are simultaneously and iteratively updated based on the loss function until the training of the preset condition is satisfied, and a trained parameter judgment model is obtained.

In 1060, adjusting the performance parameters based on the performance parameter bias.

In some embodiments, the processor may adjust the performance parameters based on the performance parameter bias. When the performance parameters are too large, the processor may adjust the performance parameters small; when the performance parameters are too small, the processor may adjust the performance parameters large. In some embodiments, the greater the probability corresponding to the performance parameters bias is, the greater the adjustment amplitude of the performance parameters is. In some embodiments, the culture fluid provision module 120 or the culture module 110 may be controlled based on the adjusted performance parameters.

In some embodiments, operations 1030-1060 may be performed once whenever the performance parameters need to be adjusted to enhance the safety and reliability of culture cultivation and reduce the possibility of problems with the culture.

Since the growth condition of the culture is determined based on the obtained image of the culture through a preset algorithm including a machine learning model, which is trained by labeling images of cultures with different growth conditions. Therefore, there exists the following possible scenario: a first difference between the growth condition of the culture and the expected condition is found to be large at a first cultivation time point, thus the performance parameters are adjusted. After the adjustment, at the second cultivation time point, the growth condition of the culture is better compared to the growth condition at the first cultivation time point before the adjustment, but a second difference relative to the expected condition corresponding to the second cultivation time point becomes larger compared to the first difference. It may be that since the growth condition of the culture at the first cultivation time point before the adjustment is lagged far behind the expected condition, the growth of the expected condition is greater than the growth of the culture due to the adjustment of the performance parameter in the process from the first cultivation time point to the second cultivation time point, which results in the growth of the culture at the second cultivation time point exceeding the growth of the culture at the first cultivation time point. However, a difference between the growth condition at the second cultivation time point and the preset condition at the second cultivation time point is greater than a difference between the growth condition at the first cultivation time point and the preset condition at the first cultivation time point. In this case, if the performance parameters continue to be adjusted after the second cultivation time point, it may lead to problems with the cultivation of the culture. For example, when the performance parameters include a culture fluid provision rate of the culture fluid provision module 120, in order to cause the growth of the culture to catch up with the preset situation, the culture fluid provision rate of the culture fluid provision module 120 may continue to be increased after the second cultivation time point, and too large culture fluid provision rate may lead to a problem of over-nutrition. Problems caused by over-nutrition include that (1) over-nutrition may stimulate rapid cell proliferation, which may lead to excessive cell division and the formation of cell piles, further causing the cells to lose their original morphology and characteristics become irregular, affecting the accuracy and reliability of the study; (2) over-nutrition may cause cells to lose normal growth regulation and increase unrestricted cell proliferation, which in turn may trigger cell carcinogenesis and the formation of tumor-like structures; (3) over-nutrition may inhibit apoptosis (programmed cell death), which is carried out by cells under normal physiological conditions to maintain tissue homeostasis, leading to prolonged cell survival, which in turn may interfere with cell physiological processes and the interpretation of experimental results; (4) over-nutrition may also lead to resource constraints, e.g., excess glucose may lead to the production of acidic metabolites by the cells, which reduces the pH of the culture medium, thus affecting the growth and metabolism of the cells; (5) cells may face higher metabolic burdens under high nutrient conditions resulting from over-nutrition, and may be prone to mutations or unstable gene expression, which in turn may interfere with the results of the experiment and cause uncontrolled changes in the cells in in vitro culture. The above problems may be well avoided by the operation of operations 1030-1060.

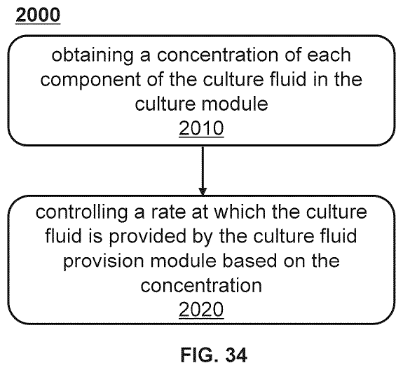

In some embodiments, referring to FIG. 34, the method for controlling the in vitro life culture system 100 may include a process 2000, and the process 2000 may include the following operations.

In 2010, obtaining a concentration of each component of the culture fluid in the culture module.

In some embodiments, the processor may obtain the concentration of each component of the culture fluid in the culture module 110. In some embodiments, a concentration detection unit may be provided in the culture chamber 2 of the culture module 110 for detecting the concentration of each component of the culture fluid in the culture chamber 2. In some embodiments, the concentration detection unit may use a sample detection or an in-situ detection to detect. In some embodiments, a detection manner of the concentration detection unit may be the same as that of the metabolite concentration detection unit described elsewhere in the present disclosure, which is not repeated herein.

In 2020, controlling a rate at which the culture fluid is provided by the culture fluid provision module based on the concentration.

In some embodiments, the processor may control the rate at which the culture fluid is provided by the culture fluid provision module 120 based on the obtained concentration of each component of the culture fluid in the culture module 110 and a preset concentration. For example, when the obtained concentration of each component of the culture fluid in the culture module 110 is higher than the preset concentration, the processor may control the culture fluid provision module 120 to slow down the rate of providing the culture fluid; when the obtained concentration of each component of the culture fluid in the culture module 110 is lower than the preset concentration, the processor may control the culture fluid provision module 120 to speed up the rate of providing the culture fluid. In some embodiments, the processor may also control the rate at which the culture fluid is provided by the culture fluid provisioning module 120 based on a percentage of the obtained concentration of each component of the culture fluid in the culture module 110 to the preset concentration. For example, when the obtained concentration of each component of the culture fluid in the culture module 110 is 80% of the preset concentration, the processor may control the culture fluid provision module 120 to increase the current rate of providing the culture fluid by 20%.

In some embodiments, the processor may also control the rate at which the culture fluid is provided by the culture fluid provision module 120 based on a consumption of each component of the culture fluid in the culture chamber 2. In some embodiments, the consumption of each component of the culture fluid may be determined based on an original concentration of the component in the culture fluid and the concentration detected by the concentration detection unit. In some embodiments, the consumption may include, but is not limited to, at least one of a consumption rate and a consumption amount. In some embodiments, the processor may control the rate at which the culture fluid is provided by the culture fluid provision module 120 based on a difference between the consumption of the culture fluid in the culture chamber 2 and the preset consumption. For example, when the consumption rate and the consumption amount of the culture fluid in the culture chamber 2 are below a preset consumption rate and a preset consumption amount, the processor may control the culture fluid provision module 120 to speed up the rate of providing the culture fluid to replenish the culture fluid. As another example, when the consumption rate and the consumption amount of the culture fluid in the culture chamber 2 are higher than the preset consumption rate and the preset consumption amount, the processor may control the culture fluid provision module 120 to slow down the rate of providing culture fluid.

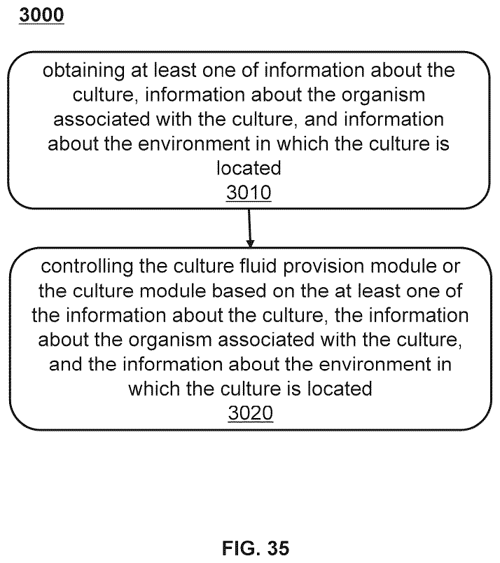

In some embodiments, referring to FIG. 35, the method for controlling the in vitro life culture system 100 may include a process 3000, and the process 3000 may include the following operations.

In 3010, obtaining at least one of information about the culture, information about the organism associated with the culture, and information about the environment in which the culture is located.

In some embodiments, the processor may obtain at least one of the information about the culture, the information about the organism associated with the culture, and the information about the environment in which the culture is located. In some embodiments, the operator may input one or more of the information about the culture, the information about the organism associated with the culture, or the information about the environment in which the culture is located to the in vitro life culture system 100 before starting the cultivation or during the cultivation process, and the processor may obtain at least one of the information based on the user's input.

The information about the culture may include, but is not limited to, information about an origin of the culture (e.g., information about an origin of an organoid: organoids differentiated from pluripotent stem cells (PSCs), induced pluripotent stem cells (iPSCs), etc., or patient-derived tumoroid organoids (PODs)), a type of the culture (e.g., types of organoids: a digestive tract organoid, a liver organoid, a pancreas organoid, a brain organoid, a kidney organoid, etc.), a cultivation time of the culture, and a cultivation degree of the culture. The information about the organism associated with the culture may include, but is not limited to, information about a sex, an age, a blood pressure, a physical health condition of the organism, and other information that may reflect individual differences among different organisms. The information about the environment in which the culture is located may include, but is not limited to, a time, a latitude and longitude, a temperature, an elevation, etc. The processor may simulate the growth environment of the organism from which the culture originated based on the information, so that the growth condition of the culture is closer to its growth condition in a real organism.

In 3020, controlling the culture fluid provision module or the culture module based on the at least one of the information about the culture, the information about the organism associated with the culture, and the information about the environment in which the culture is located.

In some embodiments, the processor may control a rate or a regularity of providing the culture fluid by the culture provision module based on the at least one of the information about the culture, the information about the organism associated with the culture, and the information about the environment in which the culture is located. In some embodiments, cultures of different origins may have different requirements for dissolved oxygen, and when it is known that the culture is a patient-derived tumor organoid based on the information about the culture, due to the fact that the core part of the tumor tissue is generally located in the hypoxic environment when growing in the organism, the processor may control the culture fluid provision module to reduce the concentration of dissolved oxygen in the mixing chamber 101 to simulate a hypoxic environment where the core part of the tumor tissue is located. The processor may determine the oxygen amount of the culture when growing in the organism based on the position at where the core part of the tumor tissue is located in the patient's body, and control the oxygen amount introduced from the gas mixing control unit 105 into the mixing chamber 101 based on the oxygen amount, so that the amount of dissolved oxygen of the culture fluid in the mixing chamber 101 is the same as or close to that oxygen amount (e.g., a difference between the amount of dissolved oxygen and the oxygen amount is within ±1%). In some embodiments, oxygen and nutrients may be delivered for different types of cultures in the organism through blood vessels, but different types of cultures may be located in different blood flow environments (e.g., a blood flow rate, a blood pressure). The processor may control the culture fluid provision module to provide the culture fluid at different rates based on the type of the culture (e.g., a liver organoid, a pancreas organoid, a brain organoid, a kidney organoid, etc.) to simulate a blood flow environment in the organism. The processor may determine the blood flow rate of the culture while it is in the patient's body based on the type of the culture, and control the power unit 5 to adjust the flow rate of the culture fluid in the culture chamber 2 to be the same as or close to the blood flow rate (e.g., a difference between the flow rate of the culture fluid and the blood flow rate is within ±1%). In some embodiments, due to differences in age, gender, physical health, or environment information (e.g., daytime or nighttime, altitude), rhythm (i.e., a heartbeat rate or a pulse) of blood supply in the different organisms may be different (120-140 beats per minute for an infant's heartbeat rate, 90-100 beats per minute for a toddler's heartbeat rate, 80-90 beats per minute for a school-age child's heartbeat rate, and 70-80 beats per minute for an adult's heartbeat rate; the heartbeat may be fast during exercise and emotional excitement during the day, while the heartbeat is slow during sleep at night; some clinical conditions, especially heart disease, may cause heartbeat change). The processor may control the culture fluid provision module 120 to introduce and discharge the culture fluid according to a blood supply regularity of the culture in the patient's body.

In some embodiments, the culture module 110 may include an oscillating drive mechanism. In some embodiments, the processor may control a motion of the oscillating drive mechanism based on at least one of the information about the culture, the information about the organism associated with the culture, and the information about the environment in which the culture is located. In some embodiments, the processor may determine that the culture is derived from an infant (e.g., 11 months of age, 24 months of age) based on the information about the organism or that it is currently daytime based on the information about the environment in which the culture is located, and the processor may control the oscillating drive mechanism to accelerate the motion, e.g., controlling the oscillating drive mechanism to cause the mixing module 7 to accelerate a shaking rate, increase a shaking amplitude, lengthen a shaking time and a shaking frequency, etc., to simulate the fast metabolism in the body of infant or the organism during the daytime. In some embodiments, the processor may determine that the culture is derived from an elderly person (e.g., 65 years old, 75 years old) based on the information about the organism or that it is currently nighttime based on the information about the environment in which the culture is located, and the processor may control the oscillating drive mechanism to slow down the motion, e.g., controlling the oscillating drive mechanism to cause the mixing module 7 to slow down the shaking rate, reduce the shaking amplitude, reduce the shaking time and shaking frequency, etc., to simulate the low metabolism in the body of the elderly person or the organism during the night time.

In some embodiments, the culture module 110 may include a temperature control unit. The temperature control unit may be used to control the temperature of the culture, e.g., raising or reducing the temperature of the culture. In some embodiments, the processor may control the temperature control unit to heat or cool the culture based on the at least one of the information about the culture, the information about the organism associated with the culture, and the information about the environment in which the culture is located. For example, the processor may control the temperature control unit to simulate a body temperature regularity of organisms of different genders and ages or a body temperature regularity of organisms in a single day based on the information about the organism associated with the culture to heat or cool the culture.

The beneficial effects of embodiments of the present disclosure are as follows.
(1) The culture fluid in the mixing chamber may be delivered into the culture chamber, and the culture fluid in the culture chamber may flow into the first exchange chamber and the culture fluid in the first exchange chamber may reflux into the culture chamber or the mixing chamber, thus realizing the perfusion cultivation of the culture. The membrane assembly may retain a portion of the components required for the growth of the culture in the culture fluid in the first exchange chamber, the waste produced by the metabolism of the culture in the culture fluid may permeate through the membrane assembly into the second exchange chamber, and the culture fluid in the first exchange chamber may reflux into the culture chamber for the culture to continue to use, realizing the circulation of the culture fluid, improving the utilization rate of the culture fluid and reducing the cultivation cost.
(2) The culture fluid in the mixing chamber may be delivered into the culture chamber, the culture fluid in the culture chamber may flow into the first exchange chamber, and the culture fluid in the first exchange chamber may reflux into the mixing chamber, thus realizing the circulation of the culture fluid, further realizing the perfusion cultivation of the culture. The membrane assembly may retain a portion of the components required for the growth of the culture in the culture fluid of the first exchange chamber, and the waste produced by the metabolism of the culture in the culture fluid may permeate through the membrane assembly into the second exchange chamber, and the culture fluid in the first exchange chamber may reflux into the mixing chamber, and then be delivered into the culture chamber for the culture to continue to use, realizing the circulation of the culture fluid, improving the utilization rate of the culture fluid, and reducing the cultivation cost. Moreover, the nutrient solution is provided to the second exchange chamber by the fluid replenishment unit, at least a portion of the components of the nutrient solution is capable of permeating through the membrane assembly into the first exchange chamber, and the components may be directionally replenished according to the consumption of the components of the culture fluid, which reduces the usage amount of the nutrient solution and the cultivation cost.
(3) The mixing chamber is capable of delivering the culture fluid to the first culture chamber, and the culture fluid in the first culture chamber or the second culture chamber is capable of refluxing into the mixing chamber, which not only realizes the perfusion cultivation of the culture, but also realizes the circulation of the culture fluid, improves the utilization rate of the culture fluid, and reduces the cultivation cost.
(4) The culture fluid in the mixing chamber may be delivered to the first culture chamber, the fluid replenishment unit may deliver the nutrient solution to the second culture chamber, the culture fluid in the first culture chamber or the second culture chamber may be delivered to the mixing chamber, and the components of the nutrient solution in the second culture chamber may permeate through the membrane assembly to the first culture chamber for the culture to absorb and utilize; the first membrane assembly may retain the components in the culture fluid required for the growth of the culture in the first culture chamber, the waste produced by the metabolism of the culture in the culture fluid may permeate through the membrane assembly into the second culture chamber, and the culture fluid in the first culture chamber or the second culture chamber may reflux into the mixing chamber, and then be delivered to the first culture chamber for the culture to continue to use, which realizes the circulation of the culture fluid, increases the utilization rate of the culture fluid, and reduces the cultivation cost. The components of the nutrient solution in the second culture chamber permeate through the membrane assembly into the first culture chamber for absorption and utilization by the culture, and the components may be directional replenished to the culture fluid according to the consumption of the components in the culture fluid, which reduces the usage amount of nutrient solution and reduces the cultivation cost.
(5) The gas mixing control unit may introduce gas into the mixing chamber to achieve the adjustment of the concentration of dissolved oxygen in the culture fluid; the temperature control unit may control the temperature of the mixing chamber and the culture chamber, the pH of the culture fluid may be adjusted by controlling the amount of nutrient solution and adding alkaline solution, the temperature, pH and the concentration of dissolved oxygen of the culture fluid may be controlled in the mixing chamber, so that the system is free from the constraint of the carbon dioxide incubator, and the scale of the cultivation may be expanded arbitrarily.
(6) The culture module is provided with a temperature control assembly, which may quickly and accurately adjust the temperature of the culture chamber to the physiological temperature of the culture or the liquefaction temperature of the matrix gel, facilitating the cultivation of the culture and taking the culture from the culture chamber after the cultivation of the culture is completed.
(7) A plurality of sub-chambers are provided in the culture chamber, and the plurality of sub-chambers may filter the culture fluid in the culture chamber to reduce the metabolite concentration in the culture fluid, causing the culture fluid in the culture chamber to be circulated and utilized, which greatly saves costs.
(8) The mixing module is provided in the culture module, which may make the contact between the culture fluid in the culture chamber and the culture more adequate, increasing the growth efficiency of the culture and improving the growth of the culture.
(9) The exchange unit provided in the culture fluid circulation module includes two or more chambers and the flow rate of the culture fluid in each chamber is different, which may effectively promote the exchange of substances in the culture fluid among the chambers, effectively improve the filtration efficiency of the culture fluid, and enable the culture fluid to be better circulated and utilized.
(10) The microscopic observation module is provided in the culture module, which may obtain an image of the culture in real time, and further obtain the growth of the culture, and then control other units or modules in the culture module, which may effectively improve the growth of the culture.

The basic concept has been described above, obviously, for those skilled in the art, the above detailed disclosure is only an example, and does not constitute a limitation to the present disclosure. Although not explicitly stated here, those skilled in the art may make various modifications, improvements and amendments to the present disclosure. These alterations, improvements, and modifications are intended to be suggested by this disclosure, and are within the spirit and scope of the exemplary embodiments of this disclosure.

Moreover, certain terminology has been used to describe embodiments of the present disclosure. For example, “one embodiment”, “an embodiment”, and/or “some embodiments” refer to a certain feature, structure or characteristic related to at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. In addition, some features, structures, or features in the present disclosure of one or more embodiments may be appropriately combined.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Although the numerical ranges and parameters used in some embodiments of the present disclosure to confirm the breadth of the scope are approximate values, in specific embodiments, such numerical values are set as precisely as practicable.

The entire contents of each patent, patent application, patent application publication, and other material, such as article, book, specification, publication, document, etc., cited in the present disclosure are hereby incorporated by reference into the present disclosure. Application history documents that are inconsistent with or conflict with the content of the present disclosure are excluded, as are documents (currently or hereafter appended to the present disclosure) that limit the broadest scope of the claims of the present disclosure. It should be noted that if there is any inconsistency or conflict between the descriptions, definitions, and/or terms used in the attached materials of the present disclosure and the contents of the present disclosure, the descriptions, definitions and/or terms used in the present disclosure shall prevail.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. An in vitro life culture system, comprising:
a culture module used to cultivate a culture, the culture module including at least a culture chamber for holding a culture fluid;
a culture fluid provision module used to provide the culture fluid to the culture module; and
a fluid output module used to discharge the culture fluid from the culture chamber.

2. The in vitro life culture system of claim 1, further comprising a culture fluid circulation module used to achieve a circulation of the culture fluid in the culture chamber.

3. The in vitro life culture system of claim 2, wherein the culture fluid circulation module includes a first exchange unit located outside the culture chamber, the first exchange unit being configured to receive the culture fluid flowing out of the culture chamber and perform a component exchange on the culture fluid.

4. The in vitro life culture system of claim 3, wherein the first exchange unit includes a first exchange chamber, a second exchange chamber, and a membrane assembly provided between the first exchange chamber and the second exchange chamber, the membrane assembly being used to retain or permeate at least a portion of components in the culture fluid.

5-8. (canceled)

9. The in vitro life culture system of claim 4, wherein the culture fluid circulation module further includes a power unit, the power unit being used to control at least one of a flow rate of the culture fluid in the first exchange chamber or a flow rate of the culture fluid in the second exchange chamber, wherein the flow rate of the culture fluid in the first exchange chamber is lower than the flow rate of the culture fluid in the second exchange chamber.

10. (canceled)

11. The in vitro life culture system of claim 4, wherein the culture fluid circulation module further includes a fluid replenishment unit used to deliver one or more components required by the culture to the first exchange unit, wherein the fluid replenishment unit is connected with the second exchange chamber.

12. (canceled)

13. The in vitro life culture system of claim 2, wherein the culture fluid circulation module includes a second exchange unit located in the culture chamber, the second exchange unit being used to perform a component exchange on the culture fluid in the culture chamber and circulate at least a portion of components of the culture fluid, wherein the second exchange unit includes a first culture chamber in the culture chamber, a second culture chamber in the culture chamber, and a first membrane assembly located between the first culture chamber and the second culture chamber, at least a portion of the components of the culture fluid being capable of passing through the first membrane assembly by permeation.

14. (canceled)

15. The in vitro life culture system of claim 13, wherein the first culture chamber includes at least one fluid inlet and at least one fluid outlet, the at least one fluid inlet being connected with the culture fluid provision module and the at least one fluid outlet being selectively connected with the culture fluid provision module.

16. (canceled)

17. (canceled)

18. The in vitro life culture system of claim 13, wherein the second culture chamber includes at least one fluid inlet and at least one fluid outlet;
wherein the at least one fluid inlet is connected with the culture fluid provision module and the at least one fluid outlet is selectively connected with the culture fluid provision module.

19. The in vitro life culture system of claim 13, wherein a third membrane assembly is provided in the first culture chamber, the third membrane assembly dividing the first culture chamber into a first sub-culture chamber and a second sub-culture chamber, wherein
the first sub-culture chamber includes a first fluid inlet and a first fluid outlet, the culture fluid provision module being connected with the first fluid inlet and the second culture chamber being connected with the first fluid outlet; and
the second sub-culture chamber includes a second fluid outlet, the second fluid outlet being connected with the collection unit.

20. The in vitro life culture system of claim 13, wherein the culture fluid circulation module includes a fluid replenishment unit, the fluid replenishment unit being used to deliver one or more components required by the culture to the second exchange unit.

21. (canceled)

22. The in vitro life culture system of claim 1, wherein the culture fluid provision module includes a mixing unit, the mixing unit including a mixing chamber and a fluid replenishment chamber, the mixing chamber being used to mix a nutrient solution and a gas to form the culture fluid and deliver the culture fluid to the culture module, and the fluid replenishment chamber being connected with the mixing chamber for delivering the nutrient solution to the mixing chamber.

23-25. (canceled)

26. The in vitro life culture system of claim 22, wherein the mixing unit further includes a gas mixing control unit connected with the mixing chamber for delivering the gas to the mixing chamber, wherein the gas mixing control unit is connected with at least one mixing chamber for controlling a gas concentration of the at least one mixing chamber.

27. (canceled)

28. The in vitro life culture system of claim 1, wherein the culture module includes a temperature control unit used to control a temperature of the culture chamber to a first temperature and control the temperature of the culture chamber to switch between the first temperature and a second temperature, wherein the first temperature corresponds to a liquefaction temperature of a support structure of the culture, the second temperature corresponds to a physiological temperature of the culture, and the first temperature is lower than the second temperature.

29-35. (canceled)

36. The in vitro life culture system of claim 1, wherein the culture module includes a microscopic observation module, the microscopic observation module includes an observation assembly, the observation assembly being used to observe the culture in the culture module.

37. The in vitro life culture system of claim 36, wherein the culture module includes a mixing module used to shake the culture fluid in the culture chamber, wherein the mixing module includes a stage and a frame, the culture chamber being placed on the stage, the stage being capable of driving the culture chamber to move.

38. (canceled)

39. The in vitro life culture system of claim 36, wherein the culture module further includes an automatic feeding module used to automatically add the culture to the culture chamber, wherein the automatic feeding module further includes an automatic feeder and an automatic feeding track, the automatic feeding track being provided on the frame and the automatic feeder being slidably provided on the automatic feeding track.

40. (canceled)

41. The in vitro life culture system of claim 1, wherein the culture module includes a sterility control module including a sterile work chamber, a filtration assembly, and a sterilization assembly, the culture chamber being at least provided in the sterile work chamber, the filtration assembly being used to filter gas passing into the sterile work chamber and the sterilization assembly being used to sterilize the sterile work chamber.

42. (canceled)

43. A method for controlling an in vitro life culture system, wherein the in vitro life culture system comprises a culture module used to cultivate a culture, the culture module including at least a culture chamber for holding a culture fluid; a culture fluid provision module used to provide the culture fluid to the culture module; and a fluid output module used to discharge the culture fluid from the culture chamber;
the method comprising:
obtaining a growth condition of the culture in the culture module; and
controlling the culture fluid provision module or the culture module based on the growth condition, wherein the obtaining the growth condition of the culture in the culture module includes:
obtaining an image of the culture by a microscopic observation module; and
determining the growth condition of the culture based on the image and a preset algorithm.

44-48. (canceled)

49. A method for controlling an in vitro life culture system, wherein the in vitro life culture system comprises a culture module used to cultivate a culture, the culture module including at least a culture chamber for holding a culture fluid; a culture fluid provision module used to provide the culture fluid to the culture module; and a fluid output module used to discharge the culture fluid from the culture chamber;
the method comprising:
obtaining a concentration of each component of the culture fluid in the culture module; and
controlling a rate at which the culture fluid is provided by the culture fluid provision module based on the concentration.
